(19) United States
(12) Patent Application Publication
(10) Pub. No.: US 2007/0212362 A1
(43) Pub. Date: Sep. 13, 2007

(54) USE OF IL-17 ANTIBODY FOR THE TREATMENT OF CARTILAGE DAMAGED BY OSTEOARTHRITIS

(76) Inventor: Ellen H. Filvaroff, San Francisco, CA (US)

Correspondence Address: GENENTECH, INC., 1 DNA WAY, SOUTH SAN FRANCISCO, CA, 94080, US

(21) Appl. No.: 11/559,380

(22) Filed: Nov. 13, 2006

Related U.S. Application Data
(63) Continuation of application No. 10/948,780, filed on Sep. 23, 2004.
Application No. 10/948,780 is a continuation of application No. 09/685,823, filed on Oct. 9, 2000, now abandoned.
Application No. 10/948,780 is a continuation-in-part of application No. 09/380,142, filed on Aug. 25, 1999, now abandoned.
Application No. 10/948,780 is a continuation-in-part of application No. 09/311,832, filed on May 14, 1999, now abandoned.
(60) Provisional application No. 60/192,103, filed on Mar. 24, 2000.
(60) Provisional application No. 60/113,621, filed on Dec. 23, 1998.
(60) Provisional application No. 60/085,579, filed on May 15, 1998.
(60) Provisional application No. 60/113,621, filed on Dec. 23, 1998.

(51) Int. Cl. A61K 39/395

(52) U.S. Cl. 424/158.1

(57) ABSTRACT

The present invention relates to methods for the treatment and repair of cartilage, including cartilage damaged by injury or cartilagenous disorders, including degenerative cartilagenous disorders such as arthritis, comprising the administration of IL-17 and/or LIF antagonists (e.g., anti-IL-17 and anti-LIF antibodies). Optionally, the administration may be in combination with a cartilage agent (e.g., peptide growth factor, catabolism antagonist, osteo-, synovial, anti-inflammatory factor). Alternatively, the method provides for the treatment and repair of cartilage damaged by injury or cartilagenous disorders comprising the administration of IL-17 or LIF antagonists in combination with standard surgical techniques. Alternatively, the method provides for the treatment and repair of cartilage damaged by injury or cartilagenous disorders comprising the administration of chondrocytes previously treated with an effective amount of IL-17 and/or LIF antagonist. Alternatively, the method provides for the treatment of a mammal suffering from a cartilagenous disorder, comprising the administration of a therapeutically effective amount of an IL-17 and/or LIF antagonist.

### RELATED APPLICATIONS

This application is a continuation of U.S. Ser. No. 10/948,780, filed Sep. 23, 2004, now pending, which is a continuation of U.S. Ser. No. 09/685,823, filed Oct. 9, 2000, now abandoned, which (1) claims benefit of 60/192,103 filed Mar. 24, 2000, (2) is a Continuation-in-part of 09/380,142 filed Aug. 25, 1999, now abandoned, which is a 371 of PCT/US99/10733 filed May 14, 1999, which claims benefit of 60/113,621 filed Dec. 23, 1998; and (3) is a Continuation-in-part of 09/311,832, filed May 14, 1999, now abandoned, which claims benefit of a) 60/085,679 filed May 15, 1998, and (b) 60/113,621 filed Dec. 23, 1998.

### FIELD OF THE INVENTION

The present invention relates generally to the repair of cartilage and the treatment of cartilagenous disorders, including the inhibition of the activity of IL-17 and/or leukocyte inhibitory factor (LIF).

### BACKGROUND OF THE INVENTION

Cartilagenous disorders may be broadly defined as a collection of diseases characterized by a degeneration of or metabolic abnormalities in the connective tissues, all of which are manifested by pain, stiffness and limitation of motion of the affected body parts. The origin of these disease can be pathological or as a result of trauma or injury.

While osteoarthritis (OA) and rheumatoid (RA) result from distinctly different causes, the cytokines and enzymes involved in cartilage destruction in these disorders appear to be similar. OA, also known as degenerative joint disease, is the result of a series of localized degenerative processes that affect articular cartilage and result in pain and diminished function. OA is characterized by disruption of the smooth articulating surface of cartilage, with early loss of proteoglycans (PGs) and collagens, followed by formation of clefts and fibrillation, and ultimately by full-thickness loss of cartilage. Coincident with the cartilagenous changes are alterations in peri-articular bone, including thickening and gradual exposure of the subchondral bone. Bony nodules, called osteophytes, also often develop at the periphery of the cartilage surface and occasionally grow over the adjacent eroded areas. OA symptoms include local pain at the affected joints, especially after use. With disease progression, symptoms may progress to a continuous aching sensation, local discomfort and cosmetic alterations such as deformity of the affected joint.

Unlike OA which is usually more localized, rheumatoid arthritis (RA) is a systemic inflammatory disease which first appears in the synovium of the tissues surrounding the joint space. RA is a chronic autoimmune disorder characterized by symmetrical synovitis of the joint and typically affects small and large diarthrodial joints, leading to their progressive destruction. As the disease progresses, the symptoms of RA may also include fever, weight loss, thinning of the skin, multi-organ involvement, scleritis, corneal ulcers, the formation of subcutaneous or subperiosteal nodules and premature death.

Because mature chondrocytes have little potential for replication, and since recruitment of other cell types is limited by the avascular nature of cartilage, mature cartilage tissue has limited ability to repair itself. For this reason, transplantation of cartilage tissue or isolated chondrocytes into defective joints has been used to therapeutic advantage. However, tissue transplants from donors are at risk of graft rejection as well as possible transmission of infectious diseases. Although these risks can be minimized through use of the patient's own tissue or cells, the procedure requires further surgery, the creation of a new lesion in the patient's cartilage, and expensive culturing and growing of patient-specific cells. Better healing of the lesion may be achieved if the subchondral bone is penetrated—e.g. by injury, disease or surgery—because penetration into the vasculature allows recruitment and proliferation of cells to effect repair. Unfortunately, the biochemical and mechanical properties of this newly formed fibrocartilage differ from those of normal hyaline cartilage, resulting in inadequate or altered function. Fibrocartilage does not have the same type of extracellular matrix and may thus not adhere correctly to the surrounding hyaline cartilage. For this reason, the newly synthesized fibrocartilage may be more prone to breakdown and loss than the original articular hyaline cartilage tissue.

Cartilage agents (e.g., peptide growth factors) are very significant regulators of cartilage growth and cartilage cell (chondrocyte) behavior (i.e., differentiation, migration, division and matrix synthesis or breakdown). F. S. Chen et al., Am. J. Orthop. 26: 396-406 (1997). Cartilage agents that have been previously proposed to stimulate cartilage repair include insulin-like growth factor (IGF-1), Osborn, J. Orthop. Res. 7: 35-42 (1989); Florini & Roberts, J. Gerontol. 35: 23-30 (1980); Sah et al., Arch. Biochem. Biophys. 308: 137-47 (1994); bone morphogenetic protein (BMP), Sato & Urist, Clin. Orthop. Relat. Res. 183: 180-87 (1984); Chin et al., Arthritis Rheum. Dis. 34: 314-24 (1991) and transforming growth factor beta (TGF-β), Hill & Logan, Prog. Growth Fac. Res. 4: 45-68 (1992); Guerne et al., J. Cell Physiol. 158: 476-84 (1994); Van der Kraan et al., Ann. Rheum. Dis. 51: 643-47 (1992). Treatment with cartilage agents alone, or as part of an engineered device for implantation, could in theory be used to promote in vivo repair of damaged cartilage or to promote expansion of cells ex vivo prior to transplantation.

Another method of stimulating cartilage repair is to inhibit the activity of molecules which induce cartilage destruction and/or inhibit matrix synthesis. One such molecule is the cytokine IL-1, which has detrimental effects on several tissues within the joint, including the generation of synovial inflammation and up-regulation of matrix metalloproteinases and prostaglandin expression. V. Baragi et al., J. Clin. Invest. 96: 2454-60 (1995); V. M. Baragi et al., Osteoarthritis Cartilage 5: 275-82 (1997); C. H. Evans et al., J. Leukoc. Biol. 64: 55-61 (1998); C. H. Evans and P. D. Robbins, J. Rheumatol. 24: 2061-63 (1997); R. Kang et al., Biochem. Soc. Trans. 25: 533-37 (1997); R. Kang et al., Osteoarthritis Cartilage 5: 139-43 (1997). One means of antagonizing IL-1 is through treatment with soluble IL-1 receptor antagonist (IL-1ra), a naturally occurring protein that prevents IL-1 from binding to its receptor, thereby inhibiting both direct and indirect effects of IL-1 on cartilage. Other cytokines, such as tumor necrosis factor alpha (TNF-α), interferon gamma (IFN-γ), IL-6 and IL-8 have been linked to increased activation of synovial fibroblast-like cells, chondrocytes and/or macrophages. The inhibition of these cytokines may be of therapeutic benefit in preventing inflammation and cartilage destruction. In fact, molecules which inhibit TNF-α activity have been shown to have potent beneficial effects on the joints of patients with rheumatoid arthritis.

The compound nitric oxide (NO) has also been implicated to play a substantial role in the destruction of cartilage. A. R. Amin et al., Curr. Opin. Rheum. 10: 263-268 (1998). Unlike normal joint tissue which does not produce NO unless stimulated with cytokines such as IL-1, synovial membranes or cartilage obtained from arthritic joints spontaneously produce large amounts of nitric oxide for up to 3 days after removal from the joint. High levels of nitrites are found in the synovial fluid of patents with osteo- or rheumatoid arthritis. Farrell et al., Ann. Rheum. Dis. 51: 1219-1222 (1992); Renoux et al., Osteoarthritis Cartilage 4: 175-179 (1996). Moreover, tissue explants from such patients spontaneously release high levels of nitrite in the absence of stimulation with cytokines such as IL-1. Amin et al., Curr. Opin. Rheum. 10: 263-268 (1998). Support for a causative role for nitric oxide in joint degeneration comes from studies showing the reduced arthritic progression in animals treated with agents which inhibit nitric oxide production by inhibiting nitric oxide synthase (NOS). Pelletier et al., Arthritis Rheum. 41: 1275-86 (1998); Pelletier et al., Osteoarthritis Cartilage, 7: 416-8 (1999). However, the determination of whether NO may play a positive or negative role in the progression of joint degeneration may depend upon the particular animal tested, in that in another animal model of arthritis, NOS inhibitors increased arthritic lesions. Sakiniene et al., Clin. Exp. Immunol. 110: 370-7 (1997).

Excessive nitric oxide within a damaged or diseased joint can affect not only the cells producing it, i.e., synovial cells and chondrocytes, but also leukocytes and monocyte-macrophages. In this way, NO can induce additional cytokine release, inflammation, and angiogenic activity. Amin and Abramson, Curr. Opin. Rheum. 10: 263-268 (1998). Blocking nitric oxide synthase (NOS) activity can attenuate the effects of IL-1β on matrix metalloproteinase production, aggrecan synthesis, and lactate production by chondrocytes. However, the role of NO in mediating the effect of other cytokines, such as IL-17, on cartilage matrix breakdown and synthesis has not yet been determined.

Interleukin-17 is a recently described, T cell-derived cytokine, the biological functions of which are only beginning to be understood. Spriggs et al., J. Clin. Immunol. 17: 366 (1997); Broxmeyer, H. E., J. Exp. Med. 183: 2411 (1996). When IL-17 was initially identified as a cDNA clone from a rodent T-cell lymphoma, it was recognized as having a sequence similar to an open reading frame from a primate herpes virus, Herpes virus saimiri, Rouvier et al., J. Immunol. 150: 5445 (1993), Yao et al., Immunity 3: 811 (1995) [Yao-1], Fossiez et al., J. Exp. Med. 183: 2593 (1996). Subsequently, it has been confirmed that this viral protein has many if not all of the immunostimulatory activities found for the host IL-17. Fleckenstein and Desrosiers, “Herpesvirus saimiri and herpesvirus ateles,” In The Herpesviruses, I. B. Roizman, ed, Plenum Publishing Press, New York, p. 253 (1982), Biesinger, B. I. et al., Proc. Natl. Acad. Sci. USA 89: 3116 (1992). Human IL-17 is a 20-30 kDa, disulfide linked, homodimeric protein with variable glycosylation. Yao-1, supra; Fossier et al., supra. It is encoded by a 155 amino acid open reading frame that includes an N-terminal secretion signal sequence of 19-23 amino acids. The amino acid sequence of IL-17 is only similar to the Herpes virus protein described above and does not show significant identity with the sequences of other cytokines or other known proteins.

IL-17 has been shown to be produced by primary peripheral blood CD4+ T-cells upon stimulation, but was not detected in unstimulated peripheral blood T-cells, peripheral blood cells, and EBV-transformed B-cell line, or a T-cell leukemia line. WO 00/20593. IL-17 is expressed in arthritic, but not normal joints (reviewed in Martel-Pelletier, J. et al., Front. Biosci. 4: d694-703 (1999). While expression of IL-17 is restricted, the IL-17 receptor is widely expressed, a property consistent with the pleiotropic activities of IL-17. IL-17 stimulates epithelial, endothelial, and fibroblastic cells to secrete cytokines such as IL-6, IL-8, and granulocyte-colony-stimulating factor (G-CSF), as well as prostaglandin E₂ (PGE₂). Spriggs, M. K., supra.; Broxmeyer, H. E., supra. IL-17 can sustain proliferation and preferential maturation of CD34-hemopoietic progenitors into neutrophils when cultured with fibroblasts. As such, production of IL-17 may be the key mechanism by which T-cells regulate the hematopoietic system. See, Yao, et al., J. Immunol., 155(12): 5483-5486 (1995) [Yao-2], Fossiez, et al., J. Exp. Med., 183(6): 2593-2603 (1996); Kennedy, et al., J. Interferon Cytokine Res., 16(8): 611-617 (1996).

IL-17 also stimulates the production of many other factors: TNF-α, IL-6, IL-1β in macrophages [Jovanovic et al., J. Immunol. 160: 3513 (1998)]; IL-8, the intracellular adhesion molecule (ICAM-1) in human fibroblasts; Fossiez et al., supra, Yao-2, supra; granulocyte-colony-stimulating factor (G-CSF); prostaglandin (PGE₂) from synoviocytes, Fossiez et al., supra. IL-17 potentiates bone resorption [Kotake et al., J. Clin. Invest. 103: 1345 (1999)]. IL-17 induces NO production in chondrocytes and in human osteoarthritic cartilage explants, in a manner independent of IL-1β signaling. [Attur et al., Arthritis Rheum. 40(6): 1050-53 (1997)]. Within cells, IL-17 stimulates transient Ca²⁺ influx and a reduction in [cAMP]ᵢ in human macrophages. Jovanovic et al., supra and NF-□B, as well mitogen-activated protein (MAP) kinases in fibroblasts, chondrocytes, and/or macrophages. Yao-1, supra, Jovanovic et al., supra., Shalom-Barek et al., J. Biol. Chem. 273: 27467 (1998). NF-κB regulates a number of gene products involved in cell activation and growth control. Yao et al., Immunity 3: 811-821 (1995). Through the induction of a number of responses and cytokines, IL-17 is able to mediate a wide-range of effects, mostly pro-inflammatory and hematopoietic. This has led to the suggestion that IL-17 may play a pivotal role in initiating and/or sustaining an inflammatory response. Jovanovic et al., supra.

Consistent with IL-17's wide-range of effects, the cell surface receptor for IL-17 has been found to be widely expressed in many tissues and cell types Yao et al., Cytokine 9: 794 (1997) [Yao-3]. While the amino acid sequence of the hIL-17 receptor (866 a.a.) predicts a protein with a single transmembrane domain and a long, 525 amino acid intracellular domain, the receptor sequence is unique and is not similar to that of any of the receptor from the cytokine/growth factor receptor family. This coupled with the lack of similarity of IL-17 itself to other known proteins indicates that IL-17 and its receptor may be part of a novel family of signaling proteins and receptors.

Applicants have shown herein that IL-17 also causes not only an increase in the release of proteoglycans (PGs), but also a decrease in the PG synthesis in cartilage explants as demonstrated in the examples.

There exists a great need for agents which antagonize the action of IL-17, including its induction of aggrecanase, as a means of inducing repair to cartilage damaged by disease or injury.

The cytokine leukemia inhibitory factor (LIF) is a polypeptide with a broad range of biological effects. It was originally purified from mouse cells and identified on the basis of its ability to induce differentiation in, and suppress the proliferation of, the murine monocytic leukemia cell line M1. Tomida et al., J. Biol. Chem. 259: 10978-10982 (1984); Tomida et al., FEBS Lett. 178: 291-296 (1984). Human LIF (hLIF) subsequently was shown to have comparable effects on human HL60 and U937 cells, particularly when acting in collaboration with granulocyte-macrophage (GM-CSF) or granulocyte colony stimulating factors (G-CSF). Maekawa et al., Leukemia 3:270-276 (1989).

LIF exhibits a variety of biological activities and effects on different cell types. For example, LIF stimulates osteoblast proliferation and new bone formation. Metcalf et al., Proc. Natl. Acad. Sci. 86: 5948-5952 (1989), as well as bone resorption. Abe et al., Proc. Natl. Acad. Sci. 83: 5958-5962 (1986); Reid et al., Endocrinology 126: 1416-1420 (1990), LIF stimulates liver cells to produce acute phase plasma proteins, Baumann et al., J. Immunol. 143: 1163-1167 (1989), inhibits liproprotein lipase, Mori et al., Biochem. Biophys. Res. Commun. 160: 1085-1092 (1989), stimulates neuronal differentiation and survival, Murphy et al., Proc. Natl. Acad. Sci. 88: 3498-3501 (1991), Yamamori et al., Science 246: 1412-1416 (1989), and inhibits vascular endothelial cell growth, Ferrara et al., Proc. Natl. Acad. Sci. 89: 698-702 (1992). Receptors for LIF have been found on monocyte-macrophages, osteoblasts, placental trophoblasts, and liver parenchymal cells. Hilton et al., J. Cell. Biochem. 46: 21-26 (1991); Allan et al., J. Cell Physiol. 145: 110-119 (1990); Hilton et al., Proc. Natl. Acad. Sci. 85: 5971-5975 (1988).

Depending upon its particular activity or effect, LIF has been referred to by various names, including differentiation-inducing factor (DIF, D-factor), hepatocyte-stimulating factor (HSF-II, HSF-III), melanoma-derived LPL inhibitor (MLPLI), and cholinergic neuronal differentiation factor (CDF). Hilton et al., J. Cell. Biochem. 46: 21-26 (1991). Additionally, LIF has been useful for the protection, inhibition, and prevention of the deleterious effects of reactive oxygen species, including myocardial infarcts and protection of ischemic tissues. U.S. Pat. No. 5,370,870. U.S. Pat. No. 5,837,241 describes the use of anti-LIF antibody to prevent or reduce heart hypertrophy, especially when associated with heart failure.

LIF is believed to have some role in the degeneration of cartilage because it has been detected in the inflammatory exudates of arthritic joints, and has been found to induce secretion of matrix metalloproteinases (MMPs) by chondrocytes.

More recently, LIF has been more directly implicated in the degeneration of cartilage. For example, in vitro proteoglycan synthesis is decreased in both pig and goat cartilage explants in a dose-dependent manner similar to the effect observed with IL-1, although the effect is not IL-1 dependent. Bell et al., Cytokine 7(2): 137-41 (February 1995). Joint swelling and effusion volume is also increased in the radiocarpal joints of goats treated in vivo with LIF. Carroll et al., J. Interferon Cytokine Res. 15(6): 567-73 (1995).

Similarly, known LIF antagonists appear to attenuate the negative effects of LIF. For example, Bell et al., J. Rheumatol. 27(2): 332-338 (2000) has shown that the LIF and IL-1 antagonists LIF binding protein (mLBP) and IL-ra, respectively, prevented the release of proteoglycans from pig cartilage explants induced by the presence of rheumatoid arthritic synovial fluids. The production of NO from LIF-treated osteoarthritic chondrocytes was additive when combined with IL-17; Martel-Pelletier et al., Arthritis Rheum. 42(11): 2399-2409 (1999). Soluble LIF receptor alpha or LIF binding protein (LBP) isolated from mouse serum was found to block PG resorption and/or reverse the inhibition of PG synthesis by LIF in cartilage explants. Hui et al., Cytokine 10(3): 220-226 (1998). Similarly, LBP negated the effect of LIF on joint swelling, effusion volume, leukocyte infiltration and cartilage proteoglycan catabolism during in vivo treatment of radiocarpal joints of goats with LIF. Bell et al., J. Rheumatol. 24(12): 2394-402 (1997). In human articular chondrocytes, LIF stimulates production of IL-1β, IL-6, IL-8, and production of LIF is stimulated by IL-1β and TNF-α (along with IL-6 and IL-8). Henrotin et al., Osteoarthritis Cartilage 4(3): 163-73 (1996).

Unfortunately, no good treatments for OA exist. Therefore, there is a strong need for an effective therapy to induce repair of cartilage, including cartilage damaged as a result of injury and/or disease.

As such, there is great value in the use of antagonists of LIF of IL-17, such as anti-LIF or anti-IL-17 antibodies, to induce repair of cartilage damaged by disease or injury.

### SUMMARY OF THE INVENTION

The present invention concerns a method for the treatment, repair and protection of cartilage including cartilage damaged as a result of a cartilagenous disorder resulting from disease or injury. More specifically, the invention concerns a method for the treatment, repair and protection of articular cartilage comprising administering an effective amount of an antagonist of IL-17 and/or LIF. More specifically, the method provides for administration of an antagonist of IL-17 and/or LIF that are anti-IL-17 and anti-LIF antibodies, respectively. Optionally, the cartilage is articular cartilage. Alternatively, the LIF antagonists can be LIF binding protein (LBP) and LIF receptor.

In a further embodiment, the present invention concerns a method for the treatment of a mammal suffering from a cartilagenous disorder, comprising administering to said mammal a therapeutically effective amount of an antagonist to IL-17 and/or LIF. Optionally, the cartilagenous disorder is a degenerative cartilagenous disorder. In a particular aspect, the degenerative cartilagenous disorder is arthritis, more specifically osteoarthritis or rheumatoid arthritis. In a particular aspect, the IL-17 and LIF antagonists are anti-IL-17 and anti-LIF antibodies. Optionally, the cartilage is articular cartilage. In a particular aspect, the method further comprises the combination of IL-17 and/or LIF antagonist with a standard surgical technique and/or an effective amount of at least one cartilage agent. Optionally, the IL-17 and/or LIF antagonist further comprises a carrier, excipient or stabilizer.

In a further embodiment, the present invention concerns a method for the treatment of cartilage damaged by a cartilagenous disorder comprising contacting the cartilage with an effective amount of an antagonist to IL-17 or LIF. In a specific aspect the IL-17 and LIF antagonists are anti-IL-17 and anti-LIF antibodies, respectively. In a specific aspect, the cartilage is articular cartilage. More specifically, the cartilagenous disorder is a degenerative cartilagenous disorder. In an even more specific aspect, the cartilagenous disorder is arthritis, including, e.g., rheumatoid and osteoarthritis. Alternatively, the cartilagenous disorder can result from injury, e.g., microdamage or blunt trauma, a chondral fracture, an osteochondral fracture, damage to tendons, menisci or ligaments or the result of excessive mechanical stress or other biomechanical instability resulting from an injury or obesity. In a specific aspect, the cartilage is contained within a mammal, including humans, and the amount administered to said mammal is a therapeutically effective amount. In a specific aspect, the IL-17 and LIF antagonist may be administered via injection or infusion by intravenous, intraarterial, intraperitoneal, intramuscular, intralesional, intraarticular or topical administration. Alternatively, the composition may be injected directly into the afflicted cartilagenous region or joint. In an event more specific aspect, the method may further comprise an effective amount of a cartilage agent and/or a standard surgical technique. In a specific embodiment, the IL-17 and/or LIF antagonist(s) may be adminstered prior, after and/or simultaneous to the standard cartilage surgical technique. In another specific aspect, the effective amount of IL-17 and LIF antagonist further comprises an effective amount of cartilage agent.

In a further embodiment, the present invention concerns a method for preventing cartilage damaged by a cartilagenous disorder comprising contacting the cartilage with an effective amount of an antagonist to IL-17 or LIF. In a specific aspect the IL-17 and LIF antagonists are anti-IL-17 and anti-LIF antibodies, respectively. In a specific aspect, the cartilage is articular cartilage. More specifically, the cartilagenous disorder is a degenerative cartilagenous disorder. In an even more specific aspect, the cartilagenous disorder is arthritis, including, e.g., rheumatoid and osteoarthritis. Alternatively, the cartilagenous disorder can result from injury, e.g., microdamage or blunt trauma, a chondral fracture, an osteochondral fracture, damage to tendons, menisci or ligaments or the result of excessive mechanical stress or other biomechanical instability resulting from an injury or obesity. In a specific aspect, the cartilage is contained within a mammal, including humans, and the amount administered to said mammal is a therapeutically effective amount. In a specific aspect, the IL-17 and LIF antagonist may be administered via injection or infusion by intravenous, intraarterial, intraperitoneal, intramuscular, intralesional, intraarticular or topical administration. Alternatively, the composition may be injected directly into the afflicted cartilagenous region or joint. In an event more specific aspect, the method may further comprise an effective amount of a cartilage agent and/or a standard surgical technique. In a specific embodiment, the IL-17 and/or LIF antagonist(s) may be adminstered prior, after and/or simultaneous to the standard cartilage surgical technique. In another specific aspect, the effective amount of IL-17 and LIF antagonist further comprises an effective amount of cartilage agent.

In another embodiment, the invention concerns a method of maintaining, enhancing, or promoting the growth of chondrocytes in serum-free culture by contacting the chondrocytes with an effective amount of IL-17 and/or LIF antagonist. Alternatively, the present invention concerns a method of stimulating the regeneration of or preventing the degredation of cartilage resulting from injury or cartilagenous disorder in a mammal comprising transplanting into said mammal of an effective amount of chondrocytes previously treated with an effective amount of IL-17 and/or LIF antagonist.

In a further embodiment, the present invention concerns a therapeutic kit, comprising IL-17 and/or LIF antagonists and a carrier, excipient and/or stabilizer (e.g., a buffer) in suitable packaging. The kit preferably contains instructions for using the IL-17 and/or LIF antagonist to treat cartilage damaged or to prevent the initial or continued damage to cartilage as a result of a cartilagenous disorder. Alternatively, the kit may contain instructions for using the IL-17 and/or LIF antagonist to treat a cartilagenous disorder.

In a further embodiment, the invention concerns an article of manufacture, comprising:

a container;

an instruction on the container; and

a composition comprising an active agent contained within the container;

wherein the composition is effective for treating a cartilagenous disorder, the instruction on the container indicates that the composition can be used to treat a cartilagenous disorder, and the active agent in the composition is an agent which stimulates the repair of and/or prevents the degradation of cartilage.

In a preferred aspect, the active agent is an IL-17 and/or LIF antagonist, for example, anti-IL17 and/or anti-LIF antibodies, respectively.

In a further embodiment, the present invention concerns a composition comprising an effective amount of IL-17 and LIF antagonist. In a specific aspect, the cartilage being treated is present in a mammal and the effective amount is a therapeutically effective amount. In another specific aspect, the composition further comprises an effective amount of cartilage agent, including peptide growth factors, catabolism antagonists, osteo-factors, synovial-factors and anti-inflammatory factors. In yet another specific aspect, the peptide growth factors are IGF (-1 or -2), PDGF (-AA, -AB or -BB), BMPs, FGFs, TGF-β (1-3) and EGF, the catabolism antagonists are IL-1 receptor antagonists, NO inhibitors, ICE inhibitors, agents which inhibit the activity of IL-6, IL-8, IFN-γ, TNF-α, tetracyclines and variants thereof, inhibitors of apoptosis, MMP inhibitors, aggrecanase inhibitors and inhibitors of serine and cysteine proteinases, the osteo-factors are bisphosphonates or osteoprotegerin, and the anti-inflammatory factors are anti-TNF□, soluble TNF receptors, IL-1ra, soluble IL-1 receptors and IL-10.

In a further embodiment, the present invention concerns a method for the preparation of a medicament useful for the treatment of cartilagenous disorders, including degenerative cartilagenous disorders. In a specific aspect, the degenerative cartilagenous disorder is arthritis, including rheumatoid arthritis and osteoarthritis.

### BRIEF DESCRIPTION OF THE DRAWINGS

FIG. 1 shows the induction of cartilage matrix breakdown by IL-17. Porcine articular cartilage explants were treated with IL-1α or IL-17 at various concentrations (0.1, 0.2 or 1 ng/ml), and proteoglycan breakdown (A, C) and synthesis (B, D) were measured. Data represents the average of 5 independent samples −/+SEM.

FIG. 2 shows the effect of anti-LIF antibodies or IL-1ra on proteoglycan metabolism. Porcine articular cartilage explants were treated with IL-1α (1 ng/ml) or IL-17 (1 ng/ml) alone, or in the presence of antibodies to leukemia inhibitory factor (αLIF) (1.1 μg/ml) or an interleukin 1 receptor antagonist (1ra) (0.2 μg/ml), and matrix breakdown (A) or synthesis (B) was measured. Data represents the average of 5 independent samples −/+SEM.

FIG. 3 shows the effect of IL-1 or IL-17 on nitric oxide release. (A) Porcine articular cartilage was treated with IL-1 (0.1 or 1 ng/ml), IL-17 (0.1, 1, or 5 ng/ml) or a combination of the two, and nitric oxide release was determined by the Griess reaction. (B) Bovine articular cartilage explants were treated with IL-1 or IL-17 (at 1, 10 or 50 ng/ml), and nitric oxide levels in the media were measured. Data represents the average of 5 independent samples −/+SEM.

FIG. 4 shows the effect of dexamethasone on IL-1 or IL-17-induced activities. Porcine articular cartilage explants were treated with IL-1 or IL-17 (5 ng/ml) alone or in the presence of dexamethasone DEX (10 nM). Shown are measurements of (A) nitric oxide concentration (B) proteoglycan release (C) proteoglycan synthesis. Data represents the average of 5 independent samples −/+SEM.

FIG. 5 shows the effect of nitric oxide synthase inhibitors on proteoglycan metabolism. Porcine articular cartilage explants were treated with IL-1 or IL-17 (5 ng/ml) alone or with the nitric oxide synthase inhibitors L-NIL or L-NIO (500 μM). Shown are levels of (A) nitric oxide production (B) matrix breakdown and (C) matrix synthesis. Data represents the average of 5 independent samples −/+SEM.

FIG. 6 shows the effect of an aggrecanase inhibitor on matrix metabolism. Porcine articular cartilage explants were treated with IL-1 or IL-17 (1 ng/ml) alone or in combination with actinonin (a) (10 μM), an inhibitor of aggrecanase activity. Levels of (A) proteoglycan release (B) nitric oxide production and (C) matrix synthesis were determined. Data represents the average of 5 independent samples −/+SEM.

FIG. 7 shows an analysis of aggrecan fragments released from articular cartilage explants. Bovine articular cartilage explants were treated with IL-17 (1, 10, or 50 ng/ml), IL-1β (50 ng/ml) or APMA (A) (1 mM), and the media was analyzed by Western blotting using specific antibodies recognizing neoepitopes of aggrecan which are exposed upon cleavage by MMPs (antibody 247, left panel) or by aggrecanase (antibody 71, right panel). The relatively high basal aggrecanase activity in the control may be explained by the fact that explants were cultured in serum-free media throughout the experiment. The pattern of bands in IL-1α treated samples (data not shown) was identical to those for IL-1β treated samples. Specific cleavage sites for MMPs or aggrecanase are shown (bottom) including epitopes recognized by #71 or #247 antibodies (in bold).

FIG. 8 shows the effect of interleukins on MMPs in cartilage explants. Conditioned media from explants cultures treated with various cytokines—IL-1α (α), IL-17 (17), b (IL-1β), control media (−) or with an MMP activator, APMA (A),—were analyzed by gel zymography for matrix metalloproteinase expression and activity. As shown, APMA activated MMPs as expected. However, neither IL-17 nor IL-1α induced MMP expression or activity.

FIG. 9 shows the effect of interleukins on MMP expression in cultured chondrocytes. Conditioned media from explants cultures treated with various cytokines—IL1α (α), IL-17 (17) {at various concentrations 50 ng/ml (50), 10 ng/ml (10), or 1 ng/ml (1)}, IL1β (IL-1β), control media (−) or with an MMP activator, APMA (A),—were analyzed by gel zymography for matrix metalloproteinase expression and activity. As shown, APMA activated MMPs as expected. In addition, IL-1α and IL-17 induced MMP expression.

FIG. 10 shows the in vivo effect of IL-17. Following intra-articular injection of IL-17, patellae were harvested, labelled with ³⁵S-sulfate, and proteoglycan synthesis was determined as described in the materials and methods section in the Examples. (A) Proteoglycan synthesis in IL-17 (80 ng)-treated patellae (IL-17) or in the contra-lateral, buffer injected knee (−). Each line represents results from an individual mouse. (B) Proteoglycan synthesis was measured in mice injected with IL-1 (12 ng) into the right knee (+), and buffer in the left (−), or in mice injected with IL-17 (80 ng) into the right knee (+) and buffer into the left (−).

FIG. 11 shows representative images of knee joints from animals 3 days after injection with buffer (PBS with 0.1% BSA) (A, D & G), IL-1α (B, E & H) or IL-17 (C, F & I). Panels A-F are stained with H&E and G-I with Safranin O. Joints from PBS injected animals were essentially normal. In animals treated with IL-1α and IL-17, the joints showed a moderate to severe peri-articular mixed inflammatory cell infiltrate (arrows, B&C), reactive synovitis (arrowheads, B, C, E & F), and arthritis (B, C, E & F) with adherence of intra-articular leukocytes to the articular surface (arrow, E). The articular cartilage surface in cytokine treated animals showed mild irregularity (E, F, G & H). The intensity of Safranin O staining of articular cartilage was reduced in severely inflamed joints (H&I) when compared with controls (G). Scale bar shown in A represents 100 μm in panels A-C. Scale bar in D represents 100 μm in panels D-I.

FIG. 12 shows a mouse model of rheumatoid arthritis. DBA1/LacJ mice were immunized with collagen type II. Just before disease onset (40 d after immunization), mice are treated with the test antibodies three times per week for 2 weeks. Forty days later, mice are sacrificed (see schematic at top of figure) and front (middle panel) and hind (bottom panel) paws are radiographed. During disease progression, animals are scored as described in materials and methods. Shown are two animals, at each end of the spectrum, from no pathology (0, left panel) to the most extreme phenotype seen (15, right panel).

FIG. 13 shows the effect of anti-IL17 in an RA model. Using the animal model of RA (see previous figure, and materials and methods), the effect of anti-IL-17 antibodies were compared to control, or anti-TNFα antibodies (Enbrel®). Every other day, animals are scored for inflammation, redness, the number of joints affected, and swelling. Sum score sick is the score for all the mice in a given treatment group. The change in this measure over time can give an indication of progression of the disorder over time.

FIG. 14 shows the effect of anti-LIF antibodies on human OA cartilage. Cartilage explants from the joints of OA patients were treated with anti-LIF antibodies for 5 days, and proteoglycan synthesis was measured. Shown are the mean cpm −/+SEM with eight samples per treatment group.

FIG. 15 shows the effect of IL-17 on articular cartilage. Cartilage explants were cultured with the indicated concentration of IL-17 (solid) or in the presence of IL-1α at the indicated concentration (hatched) or IL-1ra (IL-1 receptor antagonist, R&D Systems, 1 μg/ml) for 72 hours. Release of proteoglycans (PG) into the media (top panel) indicates matrix breakdown. Matrix synthesis was determined by incorporation of ³⁵S-sulphate into the tissue (bottom panel).

FIG. 16 shows the effect of IL-17 on the release of nitric oxide. Explants were treated with IL-17 (10 ng/ml) alone (left columns) or in the presence of IL-1α (10 ng/ml)(right columns). After 48 hours, media was assayed for nitrite concentration.

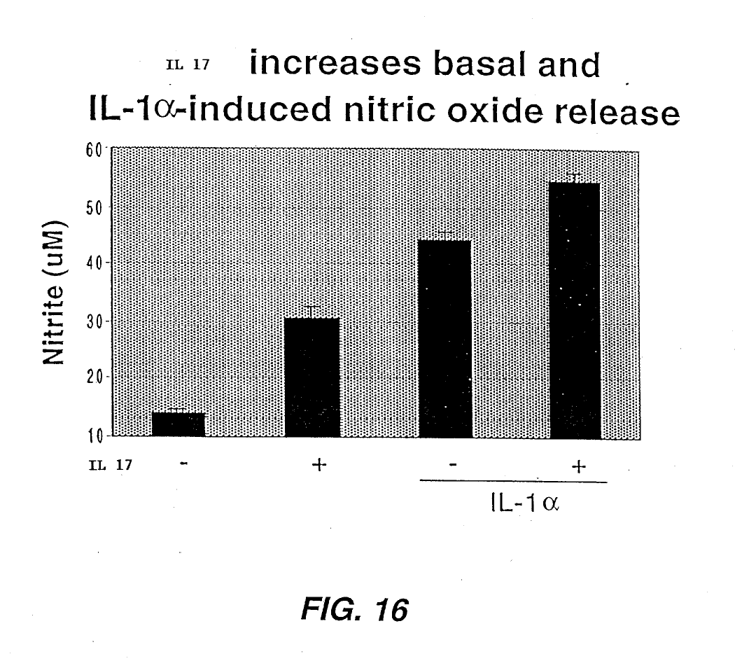

FIG. 17 shows the effect of NO on IL-17 induced changes in matrix metabolism. Explants were treated with IL-17 (5 ng/ml) alone (+) or with an irreversible inhibitor of nitric oxide synthase, NOS (L-NIO, Caymen Chemical, 0.5 mM). After 72 hours of treatment, media was assayed for (A) nitrite and (B) proteoglycans (PGs). (C) Proteoglycan synthesis was determined by incorporation of ³⁵S-sulphate into the tissue.

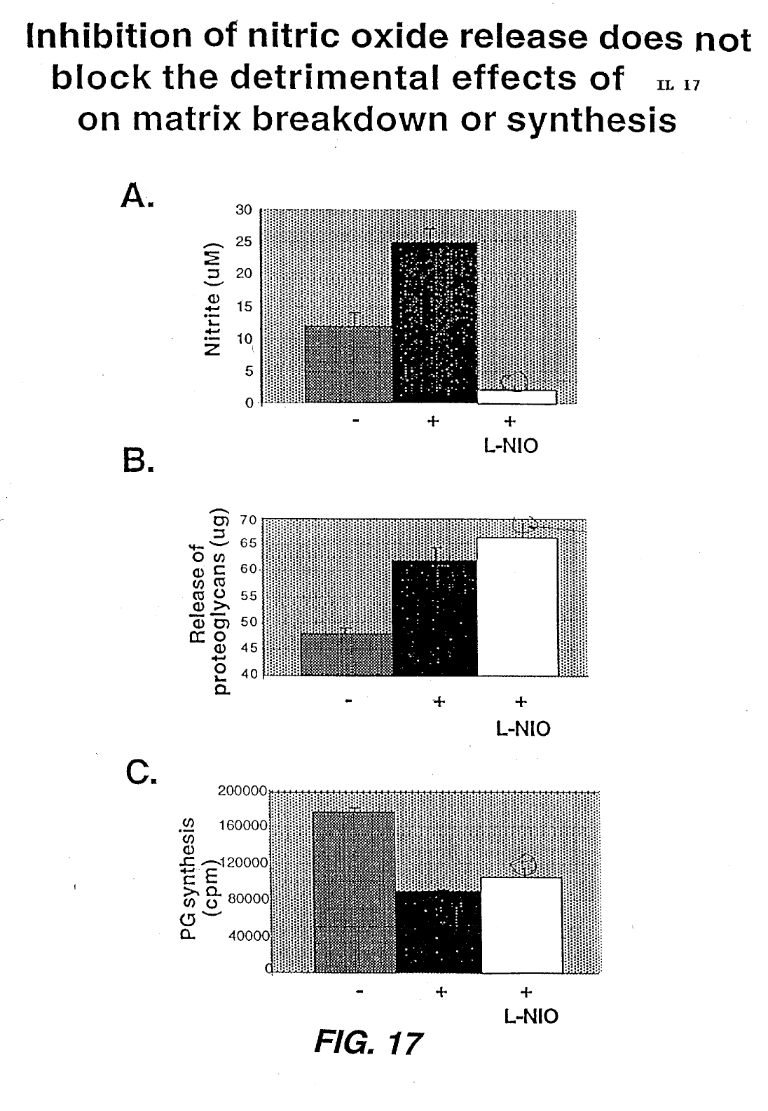

FIG. 18 shows the effect of the inhibition of NO on IL-1α-induced changes in proteoglcyan (PG) metabolism. Articular cartilage explants were treated with IL-1α (5 ng/mil) alone (+) or with inhibitors of NOS (L-NIO or L-NIL) (L-NIL, reversible NOS inhibitor, Caymen Chemical) or IL-1ra (IL-1 receptor antagonist, R&D Systems, 1 μg/ml). After 72 hours of treatment, media as assayed for (A) nitrite concentration and (B) amount of proteoglycans. (C) Matrix synthesis was determined by incorporation of ³⁵S-sulphate into the tissue.

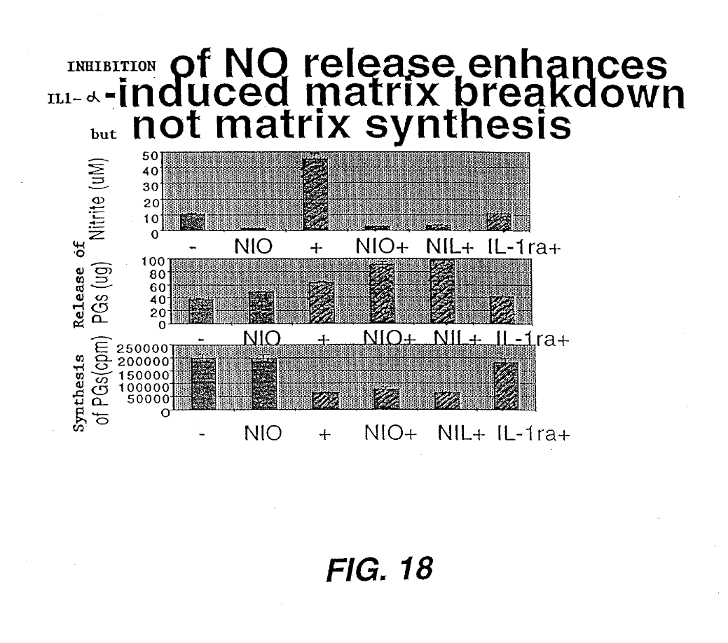

### DETAILED DESCRIPTION OF THE PREFERRED EMBODIMENTS

Osteoarthris v. Rheumatoid Arthritis:

Rheumatoid arthritis (RA) is a systemic, autoimmune, degenerative disease that causes symmetrical disruptions in the synovium of both large and small diarthroidal joints alike. As the disease progresses, symptoms of RA may include fever, weight loss, thinning of the skin, multiorgan involvement, scleritis, corneal ulcers, the formation of subcutaneous or subperiosteal nodules and premature death. In contrast to OA, RA symptoms appear during youth, extra-articular manifestations can affect any organ system, and joint destruction is symmetrical and occurs in both large and small joints alike. Extra-articular symptoms can include vasculitis, atrophy of the skin and muscle, subcutaneous nodules, lymphadenopathy, splenomegaly, leukopaenia and chronic anaemia. Furthermore, RA is heterogeneous in nature with a variable disease expression and is associated with the formation of serum rheumatoid factor in 90% of patients sometime during the course of the illness.

Interestingly, patients with RA also have a hyperactive immune system. The great majority of people with RA have a genetic susceptibility associated with increased activation of class II major histocompatibility complex molecules on monocytes and macrophages. These histocompatibility complex molecules are involved in the presentation of antigen to activated T cells bearing receptors for these class II molecules. The genetic predisposition to RA is supported by the prevalence of the highly conserved leukocyte antigen DR subtype Dw4, Dw14 and Dw15 in human patients with very severe disease.

The activated monocytes and macrophages, in interacting with the appropriate T cells, stimulate a cascade of events including further activation of additional monocytes and macrophages, T cells, B cells and endothelial cells. With the upregulation of adhesion molecules, additional mononuclear cells and polymorphonuclear cells are attracted to the inflamed joint. This influx stimulates secretion of additional chemotacetic cytokines, thereby enhancing the influx of inflammatory cells into the synovium and synovial fluid.

Osteoarthritis (OA) is a localized degenerative disease that affects articular cartilage and bone and results in pain and diminished joint function. OA may be classified into two types: primary and secondary. Primary OA refers to the spectrum of degenerative joint diseases for which no underlying etiology has been determined. Typically, the joint affected by primary OA are the interphalangeal joints of the hands, the first carpometacarpal joints, the hips, the knees, the spine, and some joints in the midfoot. Interestingly, it appears that large joints, such as the ankles, elbows and shoulders tend to be spared in primary OA. In contrast, secondary OA often occurs as a result of defined injury or trauma. Secondary arthritis can also be found in individuals with metabolic diseases such as hemochromatosis and alkaptonuria, developmental abnormalities such as developmental dysplasia of the hips (congenital dislocation of the hips) and limb-length discrepancies, obesity, inflammatory arthritis such as rheumatoid arthritis or gout, septic arthritis, and neuropathic arthritis.

OA is a progressive, degenerative disorder. The degradation associated with OA initially appears as fraying and fibrillation of the articular cartilage surface as proteoglycans are lost from the matrix. With continued joint use, surface fibrillation progresses, defects penetrate deeper into the cartilage, and pieces of cartilage tissue are lost. In addition, bone underlying the cartilage (subchondral bone) thickens, and, as cartilage is lost, bone becomes slowly exposed. With asymmetric cartilage destruction, disfigurement can occur. Bony nodules, called osteophytes, often form at the periphery of the cartilage surface and occasionally grow over the adjacent eroded areas. If the surface of these bony outgrowths is permeated, vascular outgrowth may occur and cause the formation of tissue plugs containing fibrocartilage.

Since cartilage is avascular, damage which occurs to the cartilage layer but does not penetrate to the subchondral bone, leaves the job of repair to the resident chondrocytes, which have little intrinsic potential for replication. However, when the subchondral bone is penetrated, its vascular supply allows a triphasic repair process to take place. The suboptimal cartilage which is synthesized in response to this type of damage, termed herein “fibrocartilage” because of its fibrous matrix, has suboptimal biochemical and mechanical properties, and is thus subject to further wear and destruction. In a diseased or damaged joint, increased release of metalloproteinases (MMPs) such as collagenases, gelatinases, stromelysins, aggrecanases, and other proteases, leads to further thinning and loss of cartilage. In vitro studies have shown that cytokines such as IL-1α, IL-1β, TNF-α, PDGF, GM-CSF, IFN-γ, TGF-β, LIF, IL-2 and IL-6, IL-8 can alter the activity of synovial fibroblast-like cells, macrophage, T cells, and/or osteoclasts, suggesting that these cytokines may regulate cartilage matrix turnover in vivo. As such, any of these cytokines could amplify and perpetuate the destructive cycle of joint degeneration in vivo. In fact, inhibition of IL-1 or TNF-α activity in arthritic animals and humans has been shown to be an effective way in which to at least slow the progression of arthritis. While the initiating events in RA and OA are clearly different, subsequent cartilage and bone loss in these two degenerative disorders appears to involve many of the same cytokines and proteinases.

The mechanical properties of cartilage are determined by its biochemical composition. While the collagen architecture contributes to the tensile strength and stiffness of cartilage, the compressibility (or elasticity) is due to its proteoglycan component. In healthy articular cartilage, type II collagen predominates (comprising about 90-95%), however, smaller amounts of types V, VI, IX, and XI collagen are also present. Cartilage proteoglycans (PG) include hydrodynamically large, aggregating PG, with covalently linked sulfated glycosaminoglycans, as well as hydrodynamically smaller nonaggregating PG such as decorin, biglycan and lumican.

Types of Injuries to Cartilage

Injuries to cartilage fall into three categories: (1) microdamage or blunt trauma, (2) chondral fractures, and (3) osteochondral fractures.

Microdamage to chondrocytes and cartilage matrix may be caused by a single impact, through repetitive blunt trauma, or with continuous use of a biomechanically unstable joint. In fact, metabolic and biochemical changes such as those found in the early stages of degenerative arthritis can be replicated in animal models involving repetitive loading of articular cartilage. Radin et al., Clin. Orthop. Relat. Res. 131: 288-93 (1978). Such experiments, along with the distinct pattern of cartilage loss found in arthritic joints, highlight the role that biomechanical loading plays in the loss of homeostasis and integrity of articular cartilage in disease. Radin et al., J. Orthop. Res. 2: 221-234 (1984); Radin et al., Semin. Arthritis. Rheum. (suppl. 2) 21: 12-21 (1991); Wei et al., Acta Orthop. Scand. 69: 351-357 (1998). While chondrocytes may initially be able to replenish cartilage matrix with proteoglycans at a basal rate, concurrent damage to the collagen network may increase the rate of loss and result in irreversible degeneration. Buckwalter et al., J. Am. Acad. Orthop. Surg. 2: 192-201 (1994).

Chondral fractures are characterized by disruption of the articular surface without violation of the subchondral plate. Chondrocyte necrosis at the injury site occurs, followed by increased mitotic and metabolic activity of the surviving chondrocytes bordering the injury which leads to lining of the clefts of the articular surface with fibrous tissue. The increase in chondrocyte activity is transmitory, and the repair response results in insufficient amount and quality of new matrix components.

Osteochondral fractures, the most serious of the three types of injuries, are lesions crossing the tidemark into the underlying subchondral plate. In this type of injury, the presence of subchondral vasculature elicits the three-phase response typically encountered in vascular tissues: (1) necrosis, (2) inflammation, and (3) repair. Initially the lesion fills with blood and clots. The resulting fibrin clot activates an inflammatory response and becomes vascularized repair tissue, and the various cellular components release growth factors and cytokines including transforming growth factor beta (TGF-beta), platelet-derived growth factor (PDGF), bone morphogenic proteins, and insulin-like growth factors I and II. Buckwalter et al., J. Am. Acad. Orthop. Surg. 2: 191-201 (1994).

The initial repair response associated with osteochondral fractures is characterized by recruitment, proliferation and differentiation of precursors into chondrocytes. Mesenchymal stem cells are deposited in the fibrin network, which eventually becomes a fibrocartilagenous zone. F. Shapiro et al., J. Bone Joint Surg. 75: 532-53 (1993); N. Mitchell and N. Shepard, J. Bone Joint Surg. 58: 230-33 (1976). These stem cells, which are believed to come from the underlying bone marrow rather than the adjacent articular surface, progressively differentiate into chondrocytes. At six to eight weeks after injury, the repair tissue contains chondrocyte-like cells in a matrix of proteoglycans and predominantly type II collagen, with some type I collagen. T. Furukawa et al., J. Bone Joint Surg. 62: 79-89 (1980); J. Cheung et al., Arthritis Rheum. 23: 211-19 (1980); S. O. Hjertquist & R. Lemperg, Calc. Tissue Res. 8: 54-72 (1971). However, this newly deposited matrix degenerates, and the chondroid tissue is replaced by more fibrous tissue and fibrocartilage and a shift in the synthesis of collagen from type II to type I. H. S. Cheung et al., J. Bone Joint Surg. 60: 1076-81 (1978); D. Hamerman, “Prospects for medical intervention in cartilage repair,” Joint cartilage degradation: Basic and clinical aspects, Eds. Woessner J F et al., (1993); Shapiro et al., J. Bone Joint Surg. 75: 532-53 (1993); N. Mitchell & N. Shepard, J. Bone Joint Surg. 58: 230-33 (1976); S. O. Hjertquist & R. Lemperg, Calc. Tissue Res. 8: 54-72 (1971). Early degenerative changes include surface fibrillation, depletion of proteoglycans, chondrocyte cloning and death, and vertical fissuring from the superficial to deep layers. At one year post-injury, the repair tissue is a mixture of fibrocartilage and hyaline cartilage, with a substantial amount of type I collagen, which is not found in appreciable amounts in normal articular cartilage. T. Furukawa, et al., J. Bone Joint Surg. 62: 79-89 (1980).

From a clinical viewpoint, the fibrocartilagenous repair tissue may function satisfactorily for a certain length of time. However, fibrocartilage has inferior biomechanical properties relative to that of normal hyaline cartilage. Collagen fibers are arrayed in a random orientation with a lower elastic modulus than in normal hyaline cartilage. J. Colletti et al., J. Bone Joint Surg. 54: 147-60 (1972). The permeability of the repair tissue is also elevated, thus reducing the fluid-pressure load-carrying capacity of the tissue. H. Mankin et al., “Form and Function of Articular Cartilage”, Orthopaedic Basic Science, Ed: Simon & Schuster, American Academy of Orthopeadic Surgeons, Rosemont, Ill. (1994). These changes result in increased viscoelastic deformation, making the repair tissue less able to withstand repetitive loading than normal articular cartilage. Glycosaminoglycan (GAG) levels in the cartilage adjacent to osteochondral defects have been reported to be reduced by 42% of normal values, indicating that injury leads to degeneration beyond the initial defect. Osteoarthritis Cartilage 3: 61-70 (1995).

Chondrocyte Transplantation and Survival:

The transplantation of chondrocytes, the cells responsible for secreting cartilage matrix, has also been suggested as a means of effecting cartilage repair. However, the disadvantages of allografts, e.g. the possibility of the host's immunogenic response as well as the transmission of viral and other infectious diseases, has effectively limited the scope of allogenic chondrocyte transplantation. Although these risks can be minimized by using the patient's own tissue or cells, this procedure requires further surgery, creation of a new lesion in the patient's cartilage, and expensive culturing and growing of patient-specific cells.

When cultured as monolayers on tissue culture dishes, isolated chondrocytes will de-differentiate, and with time in culture, come to resemble fibroblasts. For example, collagen production will switch from predominantly type II to type I, and cells will synthesize an increased proportion of hyaluronic acid relative to the total, glycosaminoglycan (GAG) content. W. Green, Clin. Orthop. Relat. Res. 124: 237-50 (1977). However, chondrocytes grown in collagen gels or as aggregate cultures will maintain normal morphology, proteoglycan and type II collagen synthesis as well as retain their ability to accumulate metachromatic matrix in vitro. Thus, under these conditions, chondocytes will remain relatively differentiated and phenotypically stable for up to several weeks in vitro. T. Kimura et al., Clin. Orthop. Relat. Res. 186: 231-39 (1984).

Tissue Engineering:

The difficulties and expense associated with the culturing of chondrocytes has led to the design of chondrocyte-seeded or cell-free implants for articular cartilage repair using a variety of biomaterials, including: demineralized or enzymatically treated bone, L. Dahlberg et al., J. Orthop. Res. 9: 11-19 (1991); B. C. Toolan et al., J. Biomed. Mat. Res. 41: 244-50 (1998); polylacetic acid, C. R. Chu et al., J. Biomed. Mat. Res. 29: 1147-54 (1995); polyglycolic acid, C. A. Vacanti et al., Mat. Res. Soc. Symp. Proc. 252: 367-74 (1992); hydroxyapaptite/Dacron composites, K. Messner & J. Gillquist, Biomaterials 14: 513-21 (1993); fibrin, D. A. Hendrickson et al., J. Orthop. Res. 12: 485-97 (1994); collagen gels, D. Grande et al., J. Orthop. Res. 7: 208-18 (1989), S. Wakitani et al., J. Bone Joint Surg. 71: 74-80 (1989), S. Wakitani et al., J. Bone Joint Surg. 76: 579-92 (1994); and collagen fibers, J. M. Pachence et al., “Development of a tissue analog for cartilage repair,” Tissue inducing biomaterials, Eds, L. Cima & E. Ron, Materials Research Soc. Press, Pittsburgh, Pa. (1992); B. C. Toolan et al., J. Biomed. Mat. Res. 31: 273-80 (1996). Alternative tissues employed include synovial tissue, A. G. Rothwell, Orthopedics 13: 433-42 (1990); or tissues rich in mesenchymal stem cells (e.g., bone marrow or periosteal tissue), K. Messner & J. Gillquist, Mat. Res. Soc. Symp. Proc. 252: 367-74 (1992).

Standard Cartilage Surgical Techniques:

The present method may also be administered in combination with any standard cartilage surgical technique. Standard surgical techniques are surgical procedures which are commonly employed for therapeutic manipulations of cartilage, including: cartilage shaving, abrasion chondroplasty, laser repair, debridement, chondroplasty, microfracture with or without subchondral bone penetration, mosaicplasty, cartilage cell allografts, stem cell autografts, costal cartilage grafts, chemical stimulation, electrical stimulation, perichondral autografts, periosteal autografts, cartilage scaffolds, shell (osteoarticular) autografts or allografts, or osteotomy. These techniques are described and discussed in greater detail in Frenkel et al., Front. Bioscience 4: d671-685 (1999).

Cartilage Agents:

In combination with or in lieu of tissue engineering, the administration of cartilage agents (e.g., peptide growth factors) has been considered as a way to augment cartilage repair. Peptide growth factors are very significant regulators of cartilage cell differentiation, migration, adhesion, and metabolism. F. S. Chen et al., Am J. Orthop. 26: 396-406 (1997). Because cartilage agents are soluble proteins of relative small molecular mass and are rapidly absorbed and/or degraded, a great challenge exists in making them available to cells in sufficient quantity and for sufficient duration. Secreted proteins may thus need to be incorporated into engineered, implantable devices for maximum effectiveness. The ideal delivery vehicle is biocompatible, resorbable, has the appropriate mechanical properties, and degrades into non-toxic by-products.

Several secreted peptides have the potential to induce host cartilage repair without transplantation of cells. Insulin-like growth factor (IGF-1) stimulates both matrix synthesis and cell proliferation in culture, K. Osborn. J. Orthop. Res. 7: 35-42 (1989), and insufficiency of IGF-1 may have an etiologic role in the development of osteoarthritis. R. D. Coutts, et al., Instructional Course Lect. 47: 487-94, Amer. Acad. Orthop. Surg. Rosemont, Ill. (1997). Some studies indicate that serum IGF-1 concentrations are lower in osteoarthritic patients than control groups, while other studies have found no difference. Nevertheless, both serum IGF-1 levels and chondrocyte responsiveness to IGF-1 decrease with age. J. R. Florini & S. B. Roberts, J. Gerontol. 35: 23-30 (1980). Thus, both the decreased availability of IGF-1 as well as diminished chondrocyte responsiveness to IGF-1 may contribute to cartilage homeostasis and lead to degeneration with advancing age.

IGF-1 has been proposed for the treatment or prevention of osteoarthritis. In fact, intra-articular administration of IGF-1 in combination with sodium pentosan polysulfate (a chondrocyte catabolic activity inhibitor) caused improved histological appearance, and near-normal levels of degradative enzymes (neutral metalloproteinases and collagenase), tissue inhibitors of metalloproteinase and matrix collagen. R. A. Rogachefsky, et al., Ann. N.Y. Acad. Sci. 732: 392-394 (1994). The use of IGF-1 either alone or as an adjuvant with other growth factors to stimulate cartilage regeneration has been described in WO 91/19510, WO 92/13565, U.S. Pat. No. 5,444,047, EP 434,652.

Bone morphogenetic proteins (BMPS) are members of the large transforming growth factor beta (TGF-β) family of growth factors. In vitro and in vivo studies have shown that BMP induces the differentiation of mesenchymal cells into chondrocytes. K. Sato & M. Urist, Clin. Orthop. Relat. Res. 183: 180-87 (1984). Furthermore, skeletal growth factor and cartilage-derived growth factors have synergistic effects with BMP, as the combination of these growth factors with BMP and growth hormone initiates mesenchymal cell differentiation. Subsequent proliferation of the differentiated cells are stimulated by other factors. D. J. Hill & A. Logan, Prog. Growth Fac. Res. 4: 45-68 (1992).

Transforming growth factor beta (TGF-β) is produced by osteoblasts, chondrocytes, platelets, activated lymphocytes, and other cells. R. D. Coutts et al., supra. TGF-β can have both stimulatory and inhibitory properties on matrix synthesis and cell proliferation depending on the target cell, dosage, and cell culture conditions. P. Guerne et al., J. Cell Physiol. 158: 476-84 (1994); H. Van Beuningen et al., Ann. Rheum. Dis. 52: 185-91 (1993); P. Van der Kraan et al., Ann. Rheum. Dis. 51: 643-47 (1992). Furthermore, as with IGF-1, TGF-β responsiveness is decreased with age. P. Guerne et al., J. Cell Physiol. 158: 476-84 (1994). However, TGF-β is a more potent stimulator of chondrocyte proliferation than other growth factors, including platelet-derived growth factor (PDGF), bFGF, and IGF-1 (Guerne et al., supra), and can stimulate proteoglycan production by chondrocytes. TGF-β also down-regulates the effects of cytokines which stimulate chondrocyte catabolism. Van der Kraan et al., supra. In vivo, TGF-β induces proliferation and differentiation of mesenchymal cells into chondrocytes and enhances repair of partial-thickness defects in rabbit articular cartilage. E. B. Hunziker & L. Rosenberg, Trans. Orthopaed. Res. Soc. 19: 236 (1994).

Antagonism of Cartilage Catabolism

Cartilage matrix degradation is believed to be due to cleavage of matrix molecules (proteoglycans and collagens) by proteases (reviewed in Woessner J F Jr., “Proteases of the extracellular matrix”, in Mow, V., Ratcliffe, A. (eds): Structure and Function of Articular Cartilage. Boca Raton, Fla., CRC Press, 1994 and Smith R. L., Front. In Biosci. 4:d704-712 (1999). While the key enzymes involved in matrix breakdown have not yet been clearly identified, matrix metalloproteinases (MMPs) and “aggrecanases” appear to play key roles in joint destruction. In addition, members of the serine and cysteine family of proteinases, for example the cathepsins and urokinase or tissue plasminogen activator (uPA and tPA) may also be involved. Plasmin, urokinase plasminogen activator (uPA) and tissue plasminogen activator (tPA) may play an important role in the activation pathway of the metalloproteinases. Evidence connects the closely related group of cathepsin B, L and S to matrix breakdown, and these cathepsins are somewhat increased in OA. Many cytokines, including IL-1, TNF-α and LIF induce MMP expression in chondrocytes. Induction of MMPs can be antagonized by TGF-β and is potentiated, at least in rabbits, by FGF and PDGF. As shown by animal studies, inhibitors of these proteases (MMPs and aggrecanases) may at least partially protect joint tissue from damage in vivo.

Other methods of stimulating cartilage repair include blocking the effects of molecules which are associated with cartilage destruction. For example, both IL-1 (-α and -β) and nitric oxide are substances with known catabolic effects on cartilage. The cytokine IL-1 causes cartilage breakdown, including the generation of synovial inflammation and up-regulation of matrix metalloproteinases and aggrecanases. V. Baragi, et al., J. Clin. Invest. 96: 2454-60 (1995); V. M. Baragi et al., Osteoarthritis Cartilage 5: 275-82 (1997); C. H. Evans et al., J. Leukoc. Biol. 64: 55-61 (1998); C. H Evans and P. D. Robbins, J. Rheumatol. 24: 2061-63 (1997); R. Kang et al., Biochem. Soc. Trans. 25: 533-37 (1997); R. Kang et al., Osteoarthritis Cartilage 5: 139-43 (1997). Because high levels of IL-1 are found in diseased joints and IL-1 is believed to play a pivotal role in initiation and development of arthritis, inhibition of IL-1 activity may prove to be a successful therapy. In mammals only one protease, named interleukin 1 β-convertase (ICE), can specifically generate mature, active IL-1β. Inhibition of ICE has been shown to block IL-1β production and may slow arthritic degeneration (reviewed in Martel-Pelletier J. et al., Front. Biosci. 4: d694-703). The soluble IL-1 receptor antagonist (IL-1ra), a naturally occurring protein that can inhibit the effects of IL-1 by preventing IL-1 from interacting with chondrocytes, has also been shown to be effective in animal models of arthritis and is currently being tested in humans for its ability to prevent incidence or progression of arthritis.

Nitric oxide (NO) has been implicated to play a role in the destruction of cartilage. Attur et al., Arthritis & Rheum. 40: 1050-1053 (1997); Ashok et al., Curr. Opin. Rheum. 10: 263-268 (1998). Unlike normal cartilage which does not produce NO unless stimulated with cytokines such as IL-1α, cartilage obtained from osteoarthritic joints produces large amounts of nitric oxide for over 3 days in culture despite the absence of added stimuli. Moreover, inhibition of NO production has been shown to prevent IL-1α mediated cartilage destruction and chondrocyte death as well as progression of osteoarthritis in animal models. Moreover, tissue explants from such patients spontaneously release high levels of nitrite in the absence of stimulation with cytokines such as IL-1. Amin et al., Cur. Opin. Rheum. 10: 263-268 (1998). While a conclusive determination of the positive or negative role of NO in the progression of joint determination has not yet been made, the inhibition of NO can attenuate the effects of IL-1β on matrix metalloproteinase production, aggrecan synthesis, and lactate production by chondrocytes—thus, inhibition of NO may be one way to prevent cartilage destruction.

As with IL-1α and β, TNF-α is synthesized by chondrocytes, induces matrix breakdown, inhibits matrix synthesis, and is found at high levels in arthritic joints. TNF-α also synergizes with IL-1 in terms of cartilage destruction. Inhibition of TNF-α activity, in arthritic animals and humans has been shown to inhibit progression of arthritis.

Leukemia inhibitory factor (LIF), which is synthesized by both cartilage and synovium, is present in human synovial fluids. Because LIF induces the synthesis of matrix metalloproteinases (MMPs) by chondrocytes, it may be involved in the breakdown of the cartilagenous matrix.

Interferon-gamma (IFN-γ) inhibits proteoglycan synthesis by human chondrocytes without enhancing its breakdown. Indeed, IFN-γ may suppress proteoglycan loss by inhibiting the induction of MMPs.

Interleukin 8, a potent chemotacetic cytokine for polymorphonuclear neutrophils (PMN), is synthesized by a variety of cells including monocytes/macrophages, chondrocytes and fibroblasts and is induced by TNF-α. In OA patients, IL-1β, IL-6, TNF-α and IL-8 are all found in the synovial fluid. IL-8 can enhance the release of inflammatory cytokines in human mononuclear cells, including that of IL-1β, IL-6 and TNF-α, which may further modulate the inflammatory reaction (reviewed in Martel-Pelletier J. et al., Front. Biosci. 4: d694-703).

IL-6 has also been proposed as a contributor to the OA pathological process by increasing inflammatory cells in the synovial tissue and by stimulating the proliferation of chondrocytes. In addition, IL-6 can amplify the effects of IL-1 on MMP synthesis and inhibition of proteoglycan production (reviewed in Martel-Pelletier J. et al., Front. Biosci. 4: d694-703).

Interleukin 17 upregulates production of IL-1β, TNF-α, IL-6 and MMPs in human macrophages. IL-17 also induces NO production in chondrocytes, and is expressed in arthritic, but not normal joints (reviewed in Martel-Pelletier J. et al., Front. Biosci. 4: d694-703).

Basic fibroblast growth factor (bFGF), which is synthesized by chondrocytes, can induce articular chondrocyte replication. B. C. Toolan et al., J. Biomed. Mat. Res. 41: 244-50 (1998). In explants taken from young animals, bFGF in small amounts (e.g., 3 ng/ml) stimulates synthesis and inhibits breakdown of proteoglycans, while higher levels (e.g., 30-300 ng/ml) has exactly the opposite effect (i.e., synthesis inhibition and enhanced breakdown). In adult tissues, higher doses of FGF stimulated proteoglycan, protein and collagen synthesis with no cell proliferation. R. L. Sah et al., Arch. Biochem. Biophys. 308: 137-47 (1994). bFGF also regulates cartilage homeostasis by inducing the autocrine release from chondrocytes of interleukin 1 (IL-1), a potent stimulator of catabolic behavior in cartilage. bFGF further enhances IL-1-mediated protease release, perhaps through its ability to upregulate IL-1 receptors on chondrocytes. J. E. Chin et al., Arthritis Rheum. 34: 314-24 (1991). Similarly, platelet-derived growth factor (PDGF) can potentiate the catabolic effects of IL-1 and presumably of TNF-α. However, some evidence suggests that in human cartilage bFGF and PDGF may have an anticatabolic effect; whether this phenomenon is species-specific or an effect of age remains to be determined.

While inflammation does not appear to be the initiating even in osteoarthritis, inflammation does occur in osteoarthritic joints. The inflammatory cells (i.e. monocytes, macrophages, and neutrophils) which invade the synovial lining after injury and during inflammation produce metalloproteinases as well as catabolic cyokines which can contribute to further release of degradative enzymes. Although inflammation and joint destruction do not show perfect correlation in all animal models of arthritis, agents which inhibit inflammation (e.g., IL-10) also decrease cartilage and bone pathology in arthritic animals (reviewed in Martel-Pelletier J. et al., Front. Biosci. 4: d694-703). Application of agents which inhibit inflammatory cytokines may slow OA progression by countering the local synovitis which occurs in OA patients.

Numerous studies show that members of the tetracycline family of antibiotics are effective in inhibiting collagenase and gelatinase activity. Oral administration of one of these, doxycycline, proved to decrease both collagenase and gelatinase activity in cartilage from endstage hip osteoarthritis. These data suggest that an effective oral dose of doxycycline may slow down the progression of osteoarthritis. Smith R. L., Front. Biosci. 4: d704-712.

The pathology of OA involves not only the degeneration of articular cartilage leading to eburnation of bone, but also extensive remodelling of subchondral bone resulting in the so-called sclerosis of this tissue. These bony changes are often accompanied by the formation of subchondral cysts as a result of focal resorption. Agents which inhibit bone resorption, i.e. osteoprotegerin or bisphosphonates have shown promising results in animal models of arthritis, and therefore show promise in treating cartilagenous disorders. Kong et al. Nature 402: 304-308 (1999).

I. Definitions

The term “cartilagenous disorder(s)” refers to cartilage which manifests at least one pathological condition such as metabolic derangement, increased matrix proteoglycan breakdown and/or reduced proteoglycan matrix synthesis, which occurs as a result of disease or injury. Included within the scope of “cartilagenous disorders” is “degenerative cartilagenous disorders”—a collection of disorders characterized, at least in part, by degeneration or metabolic derangement of the cartilagenous connective tissues of the body, including not only the joints or related structures, including muscles, bursae (synovial membrane), tendons and fibrous tissue, but also the growth plate. In one embodiment, the term includes “articular cartilage disorders” which are characterized by disruption of the smooth articular cartilage surface and degradation of the cartilage matrix. In a mammal, “articular cartilage disorders” are further manifested by symptoms of pain, stiffness and/or limitation of motion of the affected body parts. Under certain circumstances, an additional pathology of articular cartilage disorder includes the production of nitric oxide.

Included within the scope of “articular cartilage disorders” are osteoarthritis (OA) and rheumatoid arthritis (RA). OA defines not a single disorder, but the final common pathway of joint destruction resulting from multiple processes. OA is characterized by localized asymmetric destruction of the cartilage commensurate with palpable bony enlargements at the joint margins. OA typically affects the interphalangeal joints of the hands, the first carpometacarpal joint, the hips, the knees, the spine, and some joints in the midfoot, while large joints, such as the ankles, elbows and shoulders tend to be spared. OA can be associated with metabolic diseases such as hemochromatosis and alkaptonuria, developmental abnormalities such as developmental dysplasia of the hips (congenital dislocation of the hips), limb-length discrepancies, including trauma and inflammatory arthritides such as gout, septic arthritis, neuropathic arthritis. OA may also develop after extended biomechanical instability, such as results from a sports injury or obesity.

Rheumatoid arthritis (RA) is a systemic, chronic, autoimmune disorder characterized by symmetrical synovitis of the joint and typically affects small and large diarthroid joints alike. As RA progresses, symptoms may include fever, weight loss, thinning of the skin, multiorgan involvement, scleritis, corneal ulcers, the formation of subcutaneous or subperiosteal nodules and even premature death. The symptoms of RA often appears during youth and can include vasculitis, atrophy of the skin and muscle, subcutaneous nodules, lymphadenopathy, splenomegaly, leukopaenia and chronic anaemia.

Furthermore, the term “degenerative cartilagenous disorder” may include systemic lupus erythematosus and gout, amyloidosis or Felty's syndrome. Additionally, the term covers the cartilage degradation and destruction associated with psoriatic arthritis, acute inflammation (e.g., yersinia arthritis, pyrophosphate arthritis, gout arthritis (arthritis urica), septic arthritis), arthritis associated with trauma, inflammatory bowel disease (e.g., ulcerative colitis, Crohn's disease, regional enteritis, distal ileitis, granulomatous enteritis, regional ileitis, terminal ileitis), multiple sclerosis, diabetes (e.g., insulin-dependent and non-insulin dependent), obesity, giant cell arthritis and Sjögren's syndrome.

Examples of other immune and inflammatory diseases, at least some of which may be treatable by the methods of the invention include, juvenile chronic arthritis, spondyloarthropathies, systemic sclerosis (scleroderma), idiopathic inflammatory myopathies (dermatomyositis), systemic vasculitis, sarcoidosis, autoimmune hemolytic anemia (immune pancytopenia, paroxysmal nocturnal hemoglobinuria), autoimmune thrombocytopenia (idiopathic thrombocytopenic purpura, immune-mediated thrombocytopenia), thyroiditis (Grave's disease, Hashimoto's thyroiditis, juvenile lymphocytic thyroiditis, atrophic thyroiditis) autoimmune inflammatory diseases (e.g., allergic encephalomyelitis, multiple sclerosis, insulin-dependent diabetes mellitus, autoimmune uveoretinitis, thyrotoxicosis, autoimmune thyroid disease, pernicious anemia, autograft rejection, diabetes mellitus, immune-mediated renal disease (glomerulonephritis, tubulointerstitial nephritis)), demyelinating diseases of the central and peripheral nervous systems such as multiple sclerosis, idiopathic demyelinating polyneuropathy or Guillain-Barré syndrome, and chronic inflammatory demyelinating polyneuropathy, hepatobiliary diseases such as infectious hepatitis (hepatitis A, B, C, D, E and other non-hepatotropic viruses), autoimmune chronic active hepatitis, primary biliary cirrhosis, granulomatous hepatitis, and sclerosing cholangitis, gluten-sensitive enteropathy, and Whipple's disease, autoimmune or immune-mediated skin diseases including bullous skin diseases, erythema multiforme and contact dermatitis, psoriasis, allergic diseases such as asthma, allergic rhinitis, atopic dermatitis, food hypersensitivity and urticaria, immunologic diseases of the lung such as eosinophilic pneumonia, idiopathic pulmonary fibrosis and hypersensitivity pneumonitis, transplantation associated disease including graft rejection and graft-versus-host-disease. Infectious diseases including viral diseases such as AIDS (HIV infection), hepatitis, herpes, etc., bacterial infections, fungal infections, protozoal infections, parasitic infections and respiratory syncytial virus, human immunodeficiency virus, etc.) and allergic disorders, such as anaphylacetic hypersensitivity, asthma, allergic rhinitis, atopic dermatitis, vernal conjunctivitis, eczema, urticaria and food allergies, etc.

“Treatment” is an intervention performed with the intention of preventing the development or altering the pathology of a disorder. Accordingly, “treatment” refers to both therapeutic treatment and prophylactic or preventative measures, wherein the object is to prevent or slow down the progression of or lessen the severity of the targeted pathological condition or disorder. Those in need of treatment include those already with the disorder as well as those in which the disorder is to be prevented. In the treatment of a cartilagenous disorder, a therapeutic agent may directly decrease or increase the magnitude of response of a pathological component of the disorder, or render the disease more susceptible to treatment by other therapeutic agents, e.g., antibodies, antifungals, anti-inflammatory agents, chemotherapeutics, etc.

The term “effective amount” is at least the minimum concentration of IL-17 or LIF antagonist which causes, induces or results in either a detectable improvement or repair in damaged cartilage or provides a measurable degree of protection from the continued or induced cartilage destruction in an isolated sample of cartilage matrix (e.g., retention of proteoglycans in the matrix, inhibition of proteoglycan release from the matrix, stimulation of proteoglycan synthesis). Furthermore, a “therapeutically effective amount” is at least the minimum concentration (amount) of IL-17 of LIF antagonist administered to a mammal which would be effective in at least attenuating a pathological symptom (e.g., causing, inducing or resulting in either a detectable improvement or repair in damaged articular cartilage or causing, inducing or resulting in a measurable protection from the continued or initial cartilage destruction, improvement in range of motion, reduction in pain, etc.) which occurs as a result of injury or a cartilagenous disorder.

“Cartilage agent” may be a growth factor, cytokine, small molecule, antibody, piece of RNA or DNA, virus particle, peptide, or chemical having a beneficial effect upon cartilage, including peptide growth factors, catabolism antagonists and osteo-, synovial- or anti-inflammatory factors. Alternatively, “cartilage agent” may be a peptide growth factor—such as any of the fibroblast growth factors (e.g., FGF-1, FGF-2, . . . FGF-21, etc.), IGFs, (I and II), TGF-βs (1-3), BMPs (1-7), or members of the epidermal growth factor family such as EGF, HB-EGF, TGF-α—which could enhance the intrinsic reparative response of cartilage, for example by altering proliferation, differentiation, migration, adhesion, or matrix production by chondrocytes. Alternatively, a “cartilage agent” may be a factor which antagonizes the catabolism of cartilage (e.g., IL-1 receptor antagonist (IL-1ra), NO inhibitors, IL-1β convertase (ICE) inhibitors, factors which inhibit the activity of IL-6, IL-8, LIF, IFN-γ, TNF-α activity, tetracyclines and variants thereof, inhibitors of apoptosis, MMP inhibitors, aggrecanase inhibitors, inhibitors of serine and cysteine proteases such as cathepsins and urokinase or tissue plasminogen activator (uPA and tPA). Alternatively still, “cartilage agent” includes factors which act indirectly on cartilage by affecting the underlying bone (i.e., osteofactors, e.g., bisphosphonates, osteoprotegerin), or the surrounding synovium (i.e., synovial factors) or anti-inflammatory factors (e.g., anti-TNF-α, IL1ra, IL-10, NSAIDs). For review of cartilage agent examples, please see Martel-Pelletier et al., Front. Biosci. 4: d694-703 (1999); Hering, T. M., Front. Biosci. 4: d743-761 (1999).

“Standard surgical techniques” are surgical procedures which are commonly employed for therapeutic manipulations of cartilage, including: cartilage shaving, abrasion chondroplasty, laser repair, debridement, chondroplasty, microfracture with or without subchondral bone penetration, mosaicplasty, cartilage cell allografts, stem cell autografts, costal cartilage grafts, chemical stimulation, electrical stimulation, perichondral autografts, periosteal autografts, cartilage scaffolds, shee (osteoarticular) autografts or allografts, or osteotomy. These techniques are reviewed and described in better detail in Frenkel et al., Front. Bioscience 4: d671-685 (1999).

“Chronic” administration refers to administration in a continuous mode as opposed to an acute mode, so as to maintain the initial therapeutic effect (activity) for an extended period of time. “Intermittent” administration is treatment that is done not consecutively without interruption, but rather is cyclic in nature.

The “pathology” of a cartilagenous disorder includes any physiological phenomena that compromise the well-being of the afflicted entity. This includes, without limitation, cartilage destruction, diminished cartilage repair, abnormal or uncontrollable cell growth or differentiation, antibody production, auto-antibody production, complement production and activation, interference with the normal functioning of neighboring cells, production of cytokines or other secretory products at abnormal levels, suppression or aggravation of any inflammatory or immunological response, infiltration of inflammatory cells (neutrophilic, eosinophilic, monocytic, lymphocytic) into tissue spaces, induction of pain, or any tissue effect which results in impairment of joint function or mobility.

“Biological activity” for the purposes herein refers to the ability of IL-17 or LIF antagonists to promote the regeneration of and/or prevent the destruction of cartilage. Optionally, the cartilage is articular cartilage and the regeneration and/or destruction of the cartilage is associated with an injury or a cartilagenous disorder. For example activity may be quantified by the inhibition of proteoglcyan (PG) release from cartilage, the increase in PG synthesis in cartilage, the inhibition of the production of NO, etc.

The term “antagonist” is used in the broadest sense, and includes any molecule that partially or fully blocks, inhibits, or neutralizes a biological activity of a native sequence IL-17 or LIF polypeptide or receptor. Suitable antagonist molecules specifically include agonist or antagonist antibodies or antibody fragments, fragments or amino acid sequence variants of anti-IL-17 and anti-LIF antibodies, peptides, small organic molecules, etc. Additional examples of IL-17 and LIF antagonists include soluble IL-17 and LIF receptor, and anti-IL-17 receptor (IL-17R) and anti-LIF receptor (LIFR), respectively, and LIF binding protein. Methods for identifying antagonists of an IL-17 and LIF polypeptide may comprise contacting an IL-17 or LIF polypeptide with a candidate antagonist molecule and measuring a detectable change in one or more biological activities (blocking, inhibition, or neutralization) normally associated with the IL-17 or LIF. For example, the regeneration of and/or protection from destruction of cartilage in the presence of IL-17 or LIF, the inhibition of proteoglycan release from cartilage, the increase in proteoglycan synthesis within cartilage and the inhibition of the release of NO from cartilage.

A particular example of an IL-17 and/or LIF antagonist are anti-IL-17 and anti-LIF antibodies, respectively. A specific example of anti-IL-17 useable with the present invention is recited in U.S. Pat. No. 5,688,681 and anti-LIF antibodies are given in U.S. Pat. No. 5,837,241.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The term “antibody” (Ab) as used herein includes monoclonal antibodies, polyclonal antibodies, multispecific antibodies (e.g., bispecific antibodies), and antibody fragments, so long as they exhibit the desired biological activity. The term “immunoglobulin” (Ig) is used interchangeably with “antibody” herein.

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue, or preferably, silver stain. Isolated antibody includes the antibody in situ within recombinant cells since at least one component of the antibody's natural environment will not be present. Ordinarily, however, isolated antibody will be prepared by at least one purification step.

The basic 4-chain antibody unit is a heterotetrameric glycoprotein composed of two identical light (L) chains and two identical heavy (H) chains. An IgM antibody consists of 5 of the basic heterotetramer unit along with an additional polypeptide called a J chain, and contains 10 antigen binding sites, while IgA antibodies comprise from 2-5 of the basic 4-chain units which can polymerize to form polyvalent assemblages in combination with the J chain. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (V_{H}) followed by three constant domains (C_{H}) for each of the α and γ chains and four C_{H} domains for μ and ε isotypes. Each L chain has at the N-terminus, a variable domain (V_{L}) followed by a constant domain at its other end. The V_{L} is aligned with the V_{H} and the C_{L} is aligned with the first constant domain of the heavy chain (C_{H}1). Particular amino acid residues are believed to form an interface between the light chain and heavy chain variable domains. The pairing of a V_{H} and V_{L} together forms a single antigen-binding site. For the structure and properties of the different classes of antibodies, see e.g., Basic and Clinical Immunology, 8th Edition, Daniel P. Sties, Abba I. Terr and Tristram G. Parsolw (eds), Appleton & Lange, Norwalk, Conn. 1994, page 71 and Chapter 6.

The L chain from any vertebrate species can be assigned to one of two clearly distinct types, called kappa and lambda, based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG and IgM, having heavy chains designated α, β, ε, γ and μ, respectively. The γ and μ classes are further divided into subclasses on the basis of relatively minor differences in the CH sequence and function, e.g., humans express the following subclasses: IgG1, IgG2, IgG3, IgG4, IgA1 and IgA2.

The term “variable” refers to the fact that certain segments of the variable domains differ extensively in sequence among antibodies. The V domain mediates antigen binding and defines the specificity of a particular antibody for its particular antigen. However, the variability is not evenly distributed across the entire span of the variable domains. Instead, the V regions consist of relatively invariant stretches called framework regions (FRs) of about 15-30 amino acid residues separated by shorter regions of extreme variability called “hypervariable regions” also called “complementarity determining regions” (CDRs) that are each approximately 9-12 amino acid residues in length. The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen binding site of antibodies (see Kabat et al., Sequences of Proteins of Immunological Interest, 5th Ed. Public Health Service, National Institutes of Health, Bethesda, Md. (1991). The constant domains are not involved directly in binding an antibody to an antigen, but exhibit various effector functions, such as participation of the antibody dependent cellular cytotoxicity (ADCC).

The term “hypervariable region” (also known as “complementarity determining regions” or CDRs) when used herein refers to the amino acid residues of an antibody which are (usually three or four short regions of extreme sequence variability) within the V-region domain of an immunoglobulin which form the antigen-binding site and are the main determinants of antigen specificity. There are at least two methods for identifying the CDR residues: (1) An approach based on cross-species sequence variability (i.e., Kabat et al., Sequences of Proteins of Immunological Interest (National Institute of Health, Bethesda, Miss. 1991); and (2) An approach based on crystallographic studies of antigen-antibody complexes (Chothia, C. et al., J. Mol. Biol. 196: 901-917 (1987)). However, to the extent that two residue identification techniques define regions of overlapping, but not identical regions, they can be combined to define a hybrid CDR.

The term “monoclonal antibody” as used herein refers to an antibody obtained from a population of substantially homogeneous antibodies, i.e., the individual antibodies comprising the population are identical except for possible naturally-occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The adjective “monoclonal” is not to be construed as requiring production of the antibody by any particular method. For example, the monoclonal antibodies useful in the present invention may be prepared by the hybridoma methodology first described by Kohler et al., Nature 256: 495 (1975), or they may be made using recombinant DNA methods in bacterial or eukaryotic animal or plant cells (see, e.g., U.S. Pat. No. 4,816,567). The “monoclonal antibodies” may also be isolated from phage antibody libraries using the techniques described in Clackson et al., Nature 352: 624-628 (1991) and Marks et al., J. Mol. Biol. 222: 581-597 (1991), for example.

The monoclonal antibodies for use with the method described herein include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass. (see U.S. Pat. No. 4,816,567 and Morrison et al., Proc. Natl. Acad. Sci. USA 81: 6851-6855 (1984)). Chimeric antibodies for use herein include “primatized” antibodies comprising variable domain antigen-binding sequences derived from a non-human primate (e.g., Old World Monkey, Ape, etc.), and human constant region sequences.

An “intact” antibody is one which comprises an antigen-binding site as well as a CL and at least the heavy chain domains, C_{H}1, C_{H}2 and C_{H}3. The constant domains may be native sequence constant domains (e.g., human native sequence constant domains) or an amino acid sequence variant thereof. Preferably, the intact antibody has one or more effector functions.

An “antibody fragment” comprises a portion of an intact antibody, preferably the antigen binding and/or the variable region of the intact antibody. Examples of antibody fragments include Fab, Fab′, F(ab′)₂ and Fv fragments; diabodies; linear antibodies (see U.S. Pat. No. 5,641,870, Example 2; Zapata et al., Protein Eng. 8(10): 1057-1062 [1995]); single-chain antibody molecules and multispecific antibodies formed from antibody fragments.

Papain digestion of antibodies produced two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having different antigen-binding activity and is still capable of cross-linking antigen. Fab′ fragments differ form Fab fragments by having a few additional residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The Fc fragment comprises the carboxy-terminal portions of both H chains held together by disulfides. The effector functions of antibodies are determined by sequences in the Fc region, the region which is also recognized by Fc receptors (FcR) found on certain types of cells.

“Fv” is the minimum antibody fragment which contains a complete antigen-recognition and—binding site. This fragment consists of a dimer of one heavy- and one light-chain variable region domain in tight, non-covalent association. From the folding of these two domains emanate six hypervarible loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Single-chain Fv” also abbreviated as “sFv” or “scFv” are antibody fragments that comprise the VH and VL antibody domains connected into a single polypeptide chain. Preferably, the sFv polypeptide further comprises a polypeptide linker between the V_{H} and V_{L} domains which enables the sFv to form the desired structure for antigen binding. For a review of the sFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments prepared by constructing sFv fragments (see preceding paragraph) with short linkers (about 5-10) residues) between the V_{H} and V_{L} domains such that inter-chain but not intra-chain pairing of the V domains is achieved, thereby resulting in a bivalent fragment, i.e., a fragment having two antigen-binding sites. Bispecific diabodies are heterodimers of two “crossover” sFv fragments in which the V_{H} and V_{L} domains of the two antibodies are present on different polypeptide chains. Diabodies are described in greater detail in, for example, EP 404,097; WO 93/11161; Hollinger et al., Proc. Natl. Acad. Sci. USA 90: 6444-6448 (1993).

An antibody that “specifically binds to” or is “specific for” a particular polypeptide or an epitope on a particular polypeptide is one that binds to that particular polypeptide or epitope on a particular polypeptide without substantially binding to any other polypeptide or polypeptide epitope.

The phrase “functional fragment or analog” of an antibody is a compound having qualitative biological activity in common with a full-length antibody. For example, a functional fragment or analog of an anti-IL-17 or anti-LIF antibody is one which can bind to IL-17 or LIF, respectively, in such a manner so as to prevent or substantially reduce the ability of these molecules to bind to the receptor responsible for initiating or continuing the signaling pathway ultimately resulting in the destruction of cartilage tissue.

The term “antibody mutant” refers to an amino acid sequence variant of an antibody wherein one or more of the amino acid residues have been modified. Such mutants necessarily have less than 100% sequence identity or similarity (homology) with the amino acid sequence having at least about 75% amino acid sequence identity with the amino acid sequence of either the heavy or light chain variable domain of the antibody, alternatively at least about 80% amino acid sequence identity, alternatively at least about 85% amino acid sequence identity, alternatively at least about 90% amino acid sequence identity, alternatively at least about 95% amino acid sequence identity or alternatively at least about 96%, 97%, 98% or 99% amino acid sequence identity. In one aspect, the mutated sequences are located in the antigen binding region (e.g., hypervariable or variable region).

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

An antibody “which binds” an antigen of interest, e.g., IL-17 or LIF antigen, is one that binds the antigen with sufficient affinity such that the antibody is useful as a diagnostic and/or therapeutic agent in targetting a cell expressing the antigen, and does not significantly cross-react with other proteins. In such embodiments, the extent of binding of the antibody to CD4-IgG will be less than about 10% as determined by fluorescence activated cell sorting (FACS) analysis or radioimmunoprecipitation (RIA).

An antagonist antibody which “blocks” IL-17 and/or LIF, for example, is one which reduces or prevents the binding of IL-17 or LIF to their respective receptors. In a specific example, this can be effected by binding of the antagonist to the IL-17 or LIF ligand or to the IL-17 or LIF receptor, as evidenced by an inhibition of activity of IL-17 or LIF. The neutralization dose₅₀ (ND₅₀) will be defined as that concentration of antibody required to yield one-half maximal inhibition of the cytokine activity on a responsive cell line or tissue, when the cytokine is present at a concentration just high enough to elicit a maximum response.

Antibody “effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g., B cell receptor); and B cell activation.

“Antibody-dependent cell-mediated cytotoxicity” or ADCC refers to a form of cytotoxicity in which secreted Ig bound onto Fc receptors (FcRs) present on certain cytotoxic cells (e.g., natural killer (NK) cells, neutrophils and macrophages) enable these cytotoxic effector cells to bind specifically to an antigen-bearing target cell and subsequently kill the target cell with cytotoxins. The antibodies “arm” the cytotoxic cells and are required for killing of the target cell by this mechanism. The primary cells for mediating ACDD, NK cells, express FcγRIII only, whereas monocytes express FcγRI, FcγRII and FcγRIII. Fc expression on hematopoietic cells is summarized in Table 3 on page 464 of Ravetch and Kinet, Annu. Rev. Immunol. 9: 457-92 (1991). To assess ADCC activity of a molecule of interest, an in vitro ACDD assay, such as that described in U.S. Pat. No. 5,500,362 or 5,821,337 may be performed. Useful effector cells for such assays include peripheral blood mononuclear cells (PBMC) and natural killer (NK) cells. Alternatively, or additionally, ADCC activity of the molecule of interest may be assessed in vivo, e.g., in an animal model such as that disclosed in Clynes et al., PNAS USA 95:652-656 (1998).

“Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors, FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see M. Daëron, Annu. Rev. Immunol 15:203-234 (1997). FcRs are reviewed in Ravetch and Kinet, Annu. Rev. Immunol. 9: 457-92 (1991); Capel et al., Immunomethods 4: 25-34 (1994); and de Haas et al., J. Lab. Clin. Med. 126: 330-41 (1995). Other FcRs, including those to be identified in the future, are encompassed by the term “FcR” herein. The term also includes the neonatal receptor, FcRn, which is responsible for the transfer of maternal IgGs to the fetus. Guyer et al., J. Immunol. 117: 587 (1976) and Kim et al., J. Immunol. 24: 249 (1994).

“Human effector cells” are leukocytes which express one or more FcRs and perform effector functions. Preferably, the cells express at least FcγRIII and perform ADCC effector function. Examples of human leukocytes which mediate ADCC include peripheral blood mononuclear cells (PBMC), natural killer (NK) cells, monocytes, cytotoxic T cells and neutrophils, with PBMCs and MNK cells being preferred. The effector cells may be isolated from a native source, e.g., blood.

“Complement dependent cytotoxicity” of “CDC” refers to the lysis of a target cell in the presence of complement. Activation of the classical complement pathway is initiated by the binding of the first component of the complement system (C1q) to antibodies (of the appropriate subclass) which are bound to their cognate antigen. To assess complement activation, a CDC assay, e.g., as described in Gazzano-Santoro et al., J. Immunol. Methods 202: 163 (1996), may be performed.

“Label” as used herein refers to a detectable compound or composition which is conjugated directly or indirectly to the antibody so as to generate a “labeled” antibody. The label may be detectable by itself (e.g., radioisotope labels or fluorescent lables) or, in the case of an enzymatic label, may catalyze chemical alternation of a substrate compound or composition which is detectable.

As used herein, the term “immunoadhesin” designates antibody-like molecules which combine the binding specificity of a heterologous protein (an “adhesin”) with the effector functions of immunoglobulin constant domains. Structurally, the immunoadhesins comprise a fusion of an amino acid sequence with the desired binding specificity which is other than the antigen recognition and binding site of an antibody (i.e., is “heterologous”), and an immunoglobulin constant domain sequence. The adhesin part of an immunoadhesin molecule typically is a continguous amino acid sequence comprising at least the binding site of a receptor or a ligand. The immunoglobulin, such as IgG-1, IgG-2, IgG-3 or IgG-4 subtypes, IgA (including IgA-1 and IgA-2), IgE, IgD or IgM.

The term “mammal” as used herein refers to any mammal classified as a mammal, including humans, domestic and farm animals, and zoo, sports or pet animals, such as cattle (e.g. cows), horses, dogs, sheep, pigs, rabbits, goats, cats, etc. In a preferred embodiment of the invention, the mammal is a human.

Administration “in combination with” one or more further therapeutic agents includes simultaneous (concurrent) and consecutive administration in any order.

“Carriers” as used herein include pharmaceutically-acceptable carriers, excipients, or stabilizers which are nontoxic to the cell or mammal being exposed thereto at the dosages and concentrations employed. Often the physiologically-acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN®, polyethylene glycol (PEG), PLURONICS® and hyaluronic acid (HA).

“Solid phase” is meant to be a non-aqueous matrix to which the antibody of the present invention can adhere. Examples of solid phases encompassed herein include those formed partially or entirely of glass (e.g., controlled pore glass), polysaccharides (e.g., agarose), polyacrylamides, polystyrene, polyvinyl alcohol and silicones. In certain embodiments, depending on the context, the solid phase can comprise the well of an assay plate; in others it is a purification column (e.g., an affinity chromotagraphy column). This term also includes a discontinuous solid phase of discrete particles, such as those described in U.S. Pat. No. 4,275,149.

A “liposome” is a small vesicle composed of various types of lipids, phospholipids and/or surfactant which is useful for delivery of a drug (such as IL-17 and/or LIF polypeptide or antibody thereto) to a mammal. The components of the liposome are commonly arranged in a bilayer formation, similar to the lipid arrangement of biological membranes.

A “small molecule” is defined herein to have a molecular weight below about 500 Daltons.

The term “modulate” means to affect (e.g., either upregulate, downregulate or otherwise control) the level of a signaling pathway. Cellular processes under the control of signal transduction include, but are not limited to, transcription of specific genes, normal cellular functions, such as metabolism, proliferation, differentiation, adhesion, apoptosis and survival, as well as abnormal processes, such as transformation, blocking of differentiation and metastasis.

II. Modes for Carrying out the Invention

A. Articular Cartilage Explant Assay

In this assay, the synthetic and prophylacetic potential of a test compound on intact cartilage is described. In particular, the synthesis and breakdown of proteoglycan (PG) and nitric oxide release are measured in treated articular cartilage explants. Proteoglycans are the second largest component of the organic material in articular cartilage, Kuettner, K. E. et al., Articular Cartilage Biochemistry, Raven Press, New York, USA (1986), p. 456; Muir, J., Biochem. Soc. Trans. 11: 613-622 (1983); Hardingham, T. E., Biochem. Soc. Trans. 9: 489-497 (1981). Since proteoglycans help determine the physical and chemical properties of cartilage, the decrease in cartilage PGs which occurs during joint degeneration leads to loss of compressive stiffness and elasticity, an increase in hydraulic permeability, increased water content (swelling), and changes in the organization of other extracellular components such as collagens. Thus, PG loss is an early step in the progression of cartilagenous disorders, one which further perturbs the biomechanical and biochemical stability of the joint. PGs in articular cartilage have been extensively studied because of their likely role in skeletal growth and disease. Mow, V. C. & Ratcliffe, A., Biomaterials 13: 67-97 (1992). Proteoglycan breakdown, which is increased in diseased joints, is measured in the assays described herein by quantitating PG release into the media by articular cartilage explants using the colorimetric DMMB assay. Farndale and Buttle, Biochem. Biophys. Acta 883: 173-177 (1985). Incorporation of ³⁵S-sulfate into proteoglycans is used to measure proteoglycan synthesis.

The evidence linking interleukin-1α, IL-1α, and degenerative cartilagenous disorders is substantial. For example, high levels of IL-1α (Pelletier, J. P. et al., “Cytokines and inflammation in Cartilage Degradation” in Osteoarthritic Edition of Rheumatic Disease Clinics of North America, Eds., R. W. Moskowitz, Philadelphia, W.D. Saunders Company, 1993, pp. 545-568) and IL-1 receptors (Martel-Pelletier et al., Arthritis Rheum. 35: 530-540 (1992) have been found in diseased joints, and IL-1α induces cartilage matrix breakdown and inhibits synthesis of new matrix molecules. Baragi et al., J. Clin. Invest. 96: 2454-60 (1995); Baragi et al., Osteoarthritis Cartilage 5: 275-82 (1997); Evans et al., J. Leukoc. Biol. 64: 55-61 (1998); Evans et al., J. Rheumatol. 24: 2061-63 (1997); Kang et al., Biochem. Soc. Trans. 25: 533-37 (1997); Kang et al., Osteoarthritis Cartilage 5: 139-43 (1997). Because of the association of IL-1α with disease, the test compound is also assayed in the presence of IL-1α. The ability of the compound to not only have positive effects on cartilage, but also to counteract the catabolic effects of IL-1α is strong evidence of the protective effect exhibited by the test compound. In addition, such activity suggests that the test compound could inhibit the degradation which occurs in arthritic conditions, since catabolic events initiated by IL-1α are also induced by many other cytokines and since antagonism of IL-1α activity has been shown to reduce the progression of osteoarthritis. Arend, W. P. et al., Ann. Rev. Immunol. 16: 27-55 (1998).

The production of nitric oxide (NO) can be induced in cartilage by catabolic cytokines such as IL-1. Palmer, R M J et al., Biochem. Biophys. Res. Commun. 193: 398-405 (1993). NO has also been implicated in the joint destruction which occurs in arthritic conditions. Ashock et al., Curr. Opin. Rheum. 10: 263-268 (1998). High levels of nitrites are found in the synovial fluid of patients with osteo- or rheumatoid arthritis. Farrell et al., Ann. Rheum. Dis. 51: 1219-1222 (1992); Renoux et al., Osteoarthritis Cartilage 4: 175-179 (1996). Moreover, tissue explants from such patients spontaneously release high levels of nitrite in the absence of stimulation with cytokines such as IL-1. Amin et al., Curr. Opin. Rheum. 10: 263-268 (1998). Support for a causative role for nitric oxide in joint degeneration comes from studies showing reduced arthritic progression in animals treated with agents which inhibit nitric oxide production by inhibiting nitric oxide synthase (NOS). Pelletier et al., Arthritis Rheum. 41: 1275-86 (1998). However, the determination of whether NO may play a positive or negative role in the progression of joint degeneration may depend upon the particular animal tested, in that another animal model of arthritis, NOS inhibitors increased arthritic lesions. Sakiniene et al., Clin. Exp. Immunol. 110: 370-7 (1997).

Excessive nitric oxide within a damaged or diseased joint can affect not only the cells producing it, i.e., synovial cells and chondrocytes, but also leukocytes and monocyte-macrophages. In this way, NO can induce additional cytokine release, inflammation and angiogenic activity. Amin and Abramson, Curr. Opin. Rheum. 10: 263-268 (1998). Blocking nitric oxide synthase (NOS) activity can attenuate the effect of IL-1β on matrix metalloproteinase production, aggrecan synthesis, and lactate production by chondrocytes. The assay to measure nitric oxide production described herein is based on the principle that 2,3-diaminonapthalene (DAN) reacts with nitrite under acidic conditions to form 1-(H)-naphthotriazole, a fluorescent product. As NO is quickly metabolized into nitrite (NO₂⁻¹) and nitrate (NO₃⁻¹), detection of nitrite is one means of detecting (albeit undercounting) the actual NO produced by cartilage.

The procedures employed are described in greater detail in the examples.

B. Mouse Patellae Assay

This experiment examines the effects of the test compound on proteoglycan synthesis in the patellae (knee caps) of mice. This assay uses intact cartilage (including the underlying bone) and thus tests factors under conditions which approximate the in vivo environment of cartilage. Compounds are either added to patellae in vitro, or are injected into knee joints in vivo prior to analysis of proteoglycan synthesis in patellae ex vivo. As has been shown previously, in vivo treated patellae show distinct changes in PG synthesis ex vivo. (Van den Berg et al., Rheum. Int. 1: 165-9 (1982); Verschure, P. J. et al., Ann. Rheum. Dis. 53: 455-460 (1994); and Van de Loo et al., Arthrit. Rheum. 38: 164-172 (1995). In this model, the contralateral joint of each animal can be used as a control. The procedure is described in greater detail in the examples.

C. Mouse Model of RA

Rheumatoid arthritis (RA) is an immune disorder which appears to involve production of auto-antibodies, i.e. antibodies to endogenous proteins within the body. In fact, antibodies to a protein expressed exclusively in cartilage, namely type II collagen, are present in the synovial fluid of RA patients. Trentham, D. E et al., Arthrit. Rheum. 24: 1363-9 (1981). However, these antibodies are not necessarily the cause of the disease, but rather may be secondary to the inflammation. Injection of type II collagen into animals creates a specific immune reaction within synovial joints. Features of this “collagen-induced arthritis”, or CIA, which are similar to that found in RA patients include: erosion of cartilage and bone at joint margins, proliferative synovitis, symmetrical involvement of small and medium-sized peripheral joints in the appendicular, but not the axial, skeleton. Jamieson, T. W. et al., Invest. Radiol. 20: 324-9 (1985). Furthermore, IL-1 and TNFα appear to be involved in CIA as in RA. Joosten et al., J. Immunol. 163: 5049-5055, (1999). The model is described in greater detail in the examples.

D. Aggrecanase Assay

Aggrecan is the major proteoglycan of cartilage and largely responsible for the mechanical properties of articular cartilage. Arner et al., J. Biol. Chem. 274(10):6594-6601 (1999). Aggrecan contains two N-terminal globular domain, G1 and G2, separated by a proteolytically sensitive interglobular domain (IGD), followed by a glycosaminoglycan (GAG) attachment region and a C-terminal globular domain (G3). The G1 domain of aggrecan interacts with hyaluronic acid and link protein to form large aggregates containing multiple aggrecan monomers that are trapped within the cartilage matrix. Hardingham, T. E. & Muir, H., Biochem. Biophys. Acta 279: 401-405 (1972); Heinegard, D. & Hascall, V. C., J. Biol. Chem. 249: 4250-4256 (1974); Hardingham, T. E., Biochem. J. 177: 237-247 (1979). Aggrecan provides normal cartilage with its properties of compressibility and resilience, and is one of the first matrix components to undergo measurable loss in arthritis. This loss appears to be due to an increased rate of aggrecan degradation that can be attributed to proteolytic cleaveage within the IGD of the core protein. Cleavage within this region generates large C-terminal, GAG-containing aggrecan fragments lacking the G₁ domain which are unable to bind to hyaluronic acid and thus diffuse out of the cartilage matrix.

Cleavage of aggrecan has been shown to occur at Asn³⁴¹-Phe³⁴² and at Glu³⁷³-Ala³⁷⁴ within the interglobular domain. Matrix mellaoproteinases (MMP-1, -2, -3, -7, -8, -9 and -13) are known to cleave aggrecan in vitro at the Asn³⁴¹-Phe³⁴² site. Fosang et al., J. Biol. Chem. 266: 15579-15582 (1991); Flannery, C. R. et al., J. Biol. Chem. 267: 1008-1014 (1992); Fosang et al., Biochem. J. 295: 273-276 (1993); Fosang et al., J. Biol. Chem. 267: 19470-19474 (1992); Fosang et al., FEBS Lett. 380: 17-20 (1996). Identification of G1 fragments formed by cleavage at the Asn³⁴¹-Phe³⁴² site within human articular cartilage as well as in synovial fluids suggests a role for MMPs in proteoglycan degredation in vivo. Arner et al., supra. However, these MMPs were not responsible for the cleavage at the Glu³⁷³-Ala³⁷⁴ site.

Recently, a protease termed “aggrecanase” has been identified which cleaves aggrecan at the Glu³⁷³-Ala³⁷⁴ site. Amer et al., supra; Abbaszade et al., J. Biol. Chem. 274(33): 23443-23450 (1999). Moreover, aggrecan fragments having an N-terminus of the residues ARGSV-(SEQ ID NO:3), formed by cleavage at the Glu³⁷³-Ala³⁷⁴, site have been identified in the synovial fluids of patients with osteoarthritis, inflammatory joint disease, and joint injury. Sandy et al., J. Clin. Invest. 89:1512-1516 (1992); Lohmander, L. S. et al., Arthritis Rheum. 36: 1214-1222 (1993). Thus, it has been proposed that aggrecanase is one of the major enzymes involved in the breakdown of cartilage.

Because of the association of aggrecanase (or the by-products of its presence) with disease, IL-17 was evaluated for its ability to induce the catabolic activity of aggrecanase. The procedure examines articular cartilage explants which have been cultured in the presence of IL-17 or LIF. In addition to the amount of proteoglycans released into the media, the nature of the N terminus on these proteoglycan fragments is analyzed by Western blotting using antibodies to the neoepitopes produced by proteolytic cleavage of aggrecan by aggrecanase. The procedure is described in greater detail in the Examples.

III. Compositions and Methods of the Invention

A. IL-17 and LIF Antagonists

The present invention provides for antagonists of IL-17 and LIF and to their use in the treatment of cartilagenous disorders. Particularly preferred IL-17 and LIF antagonists are anti-IL-17 and anti-LIF antibodies, respectively. Examples of anti-IL-17 and anti-LIF antibodies which can be used with the present invention, including antibodies specific to IL-17-IgG fusion proteins can be obtained from the description provided herein. Alternatively, suitable antibodies can be obtained from R & D Systems (MAB421) and U.S. Pat. No. 5,837,241, respectively. Alternatively, antibodies raised against IL-17-IgG fusion proteins (Genentech) may also be used. Examples of soluble LIF binding which can be used with the invention are described in Hui et al., Cytokine 10(3): 220-226 (1998) and Bell et al., J. Rheumatol. 24(12): 2394-402 (1997). Examples of anti-LIF antibodies are described in U.S. Pat. No. 5,688,681 and in Kim et al., J. Immunol. Methods 156:9-17 (1992).

B. Modifications of IL-17 or LIF Polypeptide or Antibody Antagonist

Covalent modifications of IL-17 or LIF polypeptide or antibody antagonists are included within the scope of this invention. One type of covalent modification includes reacting targeted amino acid residues of a IL-17 or LIF polypeptide or antibody antagonist with an organic derivatizing agent that is capable of reacting with selected side chains or the N- or C-terminal residues of an IL-17 or LIF polypeptide or antibody antagonist. Derivatization with bifunctional agents is useful, for instance, for crosslinking IL-17 or LIF polypeptide or antibody antagonist to a water-insoluble support matrix or surface. Commonly used crosslinking agents include, e.g., 1,1-bis(diazo-acetyl)-2-phenylethane, glutaraldehyde, N-hydroxy-succinimide esters, for example, esters with 4-azidosalicylic acid, homobifunctional imidoesters, including disuccinimidyl esters such as 3,3′-dithiobis-(succinimidylproprionate), bifunctional maleimides such as bis-N-maleimido-1,8-octane and agents such as methyl-3-[(p-azidophenyl)-dithio]proprioimidate.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains, T. E. Creighton, Proteins: Structure and Molecular Properties, W.H. Freeman & Co., San Francisco, pp. 79-86 (1983), acetylation of the N-terminal amine, and amidation of any C-terminal carboxyl group.

Another type of covalent modification of the IL-17 or LIF polypeptide or antibody antagonist included within the scope of this invention comprises altering the native glycosylation pattern of the polypeptide. “Altering the native glycosylation pattern” is intended for purposes herein to mean deleting one or more carbohydrate moieties found in native sequence IL-17 or LIF polypeptide or antibody antagonist, and/or adding one or more glycosylation sites that are not present in the native sequence IL-17 or LIF polypeptide or antibody antagonist. Additionally, the phrase includes qualitative changes in the glycosylation of the native proteins, involving a change in the nature and proportions of the various carbohydrate moieties present.

Addition of glycosylation sites to IL-17 or LIF polypeptide or antibody antagonist may be accomplished by altering the amino acid sequence thereof. The alteration may be made, for example, by the addition of, or substitution by, one or more serine or threonine residues to the native sequence IL-17 or LIF polypeptide or antibody antagonist (for O-linked glycosylation sites). IL-17 or LIF polypeptide or antibody antagonist amino acid sequence may optionally be altered through changes at the DNA level, particularly by mutating the DNA encoding the IL-17 or LIF polypeptide or antibody antagonist at preselected bases such that codons are generated that will translate into the desired amino acids.

Another means of increasing the number of carbohydrate moieties on the IL-17 or LIF polypeptide or antibody antagonist is by chemical or enzymatic coupling of glycosides to the polypeptide. Such methods are described in the art, e.g., in WO 87/05330 published 11 Sep. 1987, and in Aplin and Wriston, CRC Crit. Rev. Biochem., pp. 259-306 (1981).

Removal of carbohydrate moieties present on the IL-17 or LIF polypeptide or antibody antagonist may be accomplished chemically or enzymatically or by mutational substitution of codons encoding for amino acid residues that serve as targets for glycosylation. Chemical deglycosylation techniques are known in the art and described, for instance, by Hakimuddin, et al., Arch. Biochem. Biophys., 259:52 (1987) and by Edge et al., Anal. Biochem., 118:131 (1981). Enzymatic cleavage of carbohydrate moieties on polypeptides can be achieved by the use of a variety of endo- and exo-glycosidases as described by Thotakura et al., Meth. Enzymol., 138:350 (1987).

Another type of covalent modification of IL-17 or LIF polypeptide or antibody antagonist comprises linking the IL-17 or LIF polypeptide or antibody antagonist, respectively, to one of a variety of nonproteinaceous polymers, e.g., polyethylene glycol, polypropylene glycol, or polyoxyalkylenes, in the manner set forth in U.S. Pat. No. 4,640,835; 4,496,689; 4,301,144; 4,670,417; 4,791,192; or 4,179,337.

IL-17 or LIF polypeptide or antibody antagonists of the present invention may also be modified in a way to form chimeric molecules comprising an IL-17 or LIF polypeptide or antibody antagonist, respectively, fused to another, heterologous polypeptide or amino acid sequence. In one embodiment, such a chimeric molecule comprises a fusion of an IL-17 or LIF polypeptide or antibody antagonist with a tag polypeptide which provides an epitope to which an anti-tag antibody can selectively bind. The epitope tag is generally placed at the amino- or carboxyl-terminus of the IL-17 or LIF polypeptide or antibody antagonist. The presence of such epitope-tagged forms of an IL-17 or LIF polypeptide or antibody antagonist can be detected using an antibody against the tag polypeptide. Also, provision of the epitope tag enables the IL-17 or LIF polypeptide or antibody antagonist to be readily purified by affinity purification using an anti-tag antibody or another type of affinity matrix that binds to the epitope tag.

Various tag polypeptides and their respective antibodies are well known in the art. Examples include poly-histidine (poly-his) or poly-histidine-glycine (poly-his-gly) tags; the flu HA tag polypeptide and its antibody 12CA5, Field et al., Mol. Cell. Biol., 8:2159-2165 (1988); the c-myc tag and the 8F9, 3C7, 6E10, G4, B7 and 9E10 antibodies thereto, Evan et al., Molecular and Cellular Biology, 5:3610-3616 (1985); and the Herpes Simplex virus glycoprotein D (gD) tag and its antibody, Paborsky et al., Protein Engineering, 3(6):547-553 (1990). Other tag polypeptides include the Flag-peptide, Hopp et al., BioTechnology, 6:1204-1210 (1988); the KT3 epitope peptide, Martin et al., Science, 255:192-194 (1992); an α-tubulin epitope peptide, Skinner et al., J. Biol. Chem., 266:15163-15166 (1991); and the T7 gene 10 protein peptide tag, Lutz-Freyermuth et al., Proc. Natl. Acad. Sci. USA, 87:6393-6397 (1990).

In an alternative embodiment, the chimeric molecule may comprise a fusion of an IL-17 or LIF polypeptide or antibody antagonist with an immunoglobulin or a particular region of an immunoglobulin. For a bivalent form of the chimeric molecule, such a fusion could be to the Fc region of an IgG molecule. The Ig fusions preferably include the substitution of a soluble transmembrane domain deleted or inactivated) form of an IL-17 or LIF polypeptide or antibody antagonist in place of at least one variable region within an Ig molecule. In a particularly preferred embodiment, the immunoglobulin fusion includes the hinge, CH2 and CH3, or the hinge, CH1, CH2 and CH3 regions of an IgG1 molecule. For the production of immunoglobulin fusions see also U.S. Pat. No. 5,428,130, issued Jun. 27, 1995.

In yet a further embodiment, the IL-17 or LIF polypeptide or antibody antagonist of the present invention may also be modified in a way to form a chimeric molecule comprising an IL-17 or LIF polypeptide or antibody antagonist fused to a leucine zipper. Various leucine zipper polypeptides have been described in the art. See, e.g., Landschulz et al., Science 240:1759 (1988); WO 94/10308; Hoppe et al., FEBS Letters 344:1991 (1994); Maniatis et al., Nature 341:24 (1989). It is believed that use of a leucine zipper fused to an IL-17 or LIF polypeptide or antibody antagonist may be desirable to assist in dimerizing or trimerizing soluble IL-17 or LIF polypeptide or antibody antagonist in solution. Those skilled in the art will appreciate that the leucine zipper may be fused at either the N- or C-terminal end of the IL-17 or LIF polypeptide or antibody antagonist.

C. Preparation of IL-17 or LIF Polypeptide or Antibody Antagonist

The description below relates primarily to production of IL-17 or LIF polypeptide or antibody antagonist by culturing cells transformed or transfected with a vector containing IL-17 or LIF polypeptide or antibody antagonist encoding nucleic acid. It is, of course, contemplated that alternative methods, which are well known in the art, may be employed to prepare IL-17 or LIF polypeptide or antibody antagonist. For instance, the IL-17 or LIF polypeptide or antibody antagonist sequence, or portions thereof, may be produced by direct peptide synthesis using solid-phase techniques, see, e.g., Stewart et al., Solid-Phase Peptide Synthesis, W.H. Freeman Co., San Francisco, Calif. (1969); Merrifield, J. Am. Chem. Soc., 85:2149-2154 (1963). In vitro protein synthesis may be performed using manual techniques or by automation. Automated synthesis may be accomplished, for instance, using an Applied Biosystems Peptide Synthesizer (Foster City, Calif.) using manufacturer's instructions. Various portions of IL-17 or LIF polypeptide or antibody antagonist may be chemically synthesized separately and combined using chemical or enzymatic methods to produce a full-length IL-17 or LIF polypeptide or antibody antagonist.

1. Selection and Transformation of Host Cells

Host cells are transfected or transformed with expression or cloning vectors described herein for IL-17 or LIF polypeptide or antibody antagonist production and cultured in conventional nutrient media modified as appropriate for inducing promoters, selecting transformants, or amplifying the genes encoding the desired sequences. The culture conditions, such as media, temperature, pH and the like, can be selected by the skilled artisan without undue experimentation. In general, principles, protocols, and practical techniques for maximizing the productivity of cell cultures can be found in Mammalian Cell Biotechnology: A Practical Approach, M. Butler, ed. (IRL Press, 1991) and Sambrook et al., supra.

Methods of transfection are known to the ordinarily skilled artisan, for example, CaPO₄ and electroporation. Depending on the host cell used, transformation is performed using standard techniques appropriate to such cells. The calcium treatment employing calcium chloride, as described in Sambrook et al., supra, or electroporation is generally used for prokaryotes or other cells that contain substantial cell-wall barriers. Infection with Agrobacterium tumefaciens is used for transformation of certain plant cells, as described by Shaw et al., Gene, 23:315 (1983) and WO 89/05859 published 29 Jun. 1989. For mammalian cells without such cell walls, the calcium phosphate precipitation method of Graham and van der Eb, Virology, 52:456-457 (1978) can be employed. General aspects of mammalian cell host system transformations have been described in U.S. Pat. No. 4,399,216. Transformations into yeast are typically carried out according to the method of Van Solingen et al., J. Bact., 130:946 (1977) and Hsiao et al., Proc. Natl. Acad. Sci. (USA), 76:3829 (1979). However, other methods for introducing DNA into cells, such as by nuclear microinjection, electroporation, bacterial protoplast fusion with intact cells, or polycations, e.g., polybrene, polyornithine, may also be used. For various techniques for transforming mammalian cells, see Keown et al., Methods in Enzymology, 185:527-537 (1990) and Mansour et al., Nature, 336:348-352 (1988).

Suitable host cells for cloning or expressing the nucleic acid (e.g., DNA) in the vectors herein include prokaryote, yeast, or higher eukaryote cells. Suitable prokaryotes include but are not limited to eubacteria, such as Gram-negative or Gram-positive organisms, for example, Enterobacteriaceae such as E. coli. Various E. coli strains are publicly available, such as E. coli K12 strain MM294 (ATCC 31,446); E. coli X1776 (ATCC 31,537); E. coli strain W3110 (ATCC 27,325) and K5 772 (ATCC 53,635). Other suitable prokaryotic host cells include Enterobacteriaceae such as Escherichia, e.g., E. coli, Enterobacter, Erwinia, Klebisella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD266,710, published 12 Apr. 1989), Pseudomonas such as P. aeruginosa, and Streptomyces. These examples are illustrative rather than limiting. Strain W3110 is one particularly preferred host or parent host because it is a common host strain for recombinant DNA product fermentations. Preferably, the host cell secretes minimal amounts of proteolytic enzymes. For example, strain W3110 may be modified to effect a genetic mutation in the genes encoding proteins endogenous to the host, with examples of such hosts including E. coli W3110 strain 1A2, which has the complete genotype tonA; E. coli W3110 strain 9E4, which has the complete genotype tonA ptr3; E. coli W3110 strain 27C7 (ATCC 55,244), which has the complete genotype tonA, ptr3 phoA E15 (argF-lac)169 degP ompT kan^{r}; E. coli W3110 strain 40B4, which is strain 37D6 with a non-kanamycin resistant degP deletion mutation; and an E. coli strain having mutant periplasmic protease disclosed in U.S. Pat. No. 4,946,783 issued 7 Aug. 1990. Alternatively, in vivo methods of cloning, e.g., PCR or other nucleic acid polymerase reactions, are suitable.

In addition to prokaryotes, eukaryotic microbes such as filamentous fungi or yeast are suitable cloning or expression hosts for vectors encoding IL-17 or LIF polypeptide or antibody antagonist. Saccharomyces cerevisiae is a commonly used lower eukaryotic host microorganism. Others include Schizosaccharomyces pombe, Beach and Nature, Nature 290: 140 (1981); EP 139,383 published 2 May 1995; Kluyveromyces hosts, U.S. Pat. No. 4,943,529; Fleer et al., Bio/Technology, 9: 968-975 (1991) such as e.g., K. lactis (MW98-8C, CBS683, CBS4574; Louvencourt et al., J. Bacteriol. 154(2):737-42 (1983), K. fragilis (ATCC 12,424), K. bulgaricus (ATCC 16,045), K. wickeramii (ATCC 24,178), K. waltii (ATCC 56,500), K. drosophilarum (ATCC 36,906); Van den Berg et al., Bio/Technology 8: 135 (1990); K. thermotolerans, and K. marxianus; yarrowia (EP 402,226); Pichia pastoris (EP 183,070); Sreekrishna et al., J. basic Microbiol. 28: 265-278 (1988); Candid; Trichoderma reesia (EP 244,234); Neurospora crassa, Case et al., Proc. Natl. Acad. Sci. USA 76: 5359-5263 (1979); Schwanniomyces such as Schwanniomyces occidentalis (EP 394,538 published 31 Oct. 1990); and filamentous fungi such as, e.g., Neurospora, Penicillium, Tolypocladium (WO 91/00357 published 10 Jan. 19910, and Aspergillus hosts such as A. nidulans, Balance et al., Biochem. Biophys. Res. Commun. 112: 284-289 (1983); Tilburn et al., Gene 26: 205-221 (1983); Yelton et al., Proc. Natl. Acad. Sci. USA 81: 1470-1474 (1984) and A. niger (Kelly and Hynes, EMBO J. 4: 475-479 (1985). Methylotropic yeasts are selected from the genera consisting of Hansenula, Candida, Kloeckera, Pichia, Saccharomyces, Torulopsis, and Rhodotorula. A list of specific species that are exemplary of this class of yeast may be found in C. Antony, The Biochemistry of Methylotrophs 269 (1982).

Suitable host cells for the expression of glycosylated IL-17 or LIF polypeptide or antibody antagonist are derived from multicellular organisms. Examples of invertebrate cells include insect cells such as Drosophila S2 and Spodoptera Sf9, Spodoptera high5 as well as plant cells. Examples of useful mammalian host cell lines include Chinese hamster ovary (CHO) and COS cells. More specific examples include monkey kidney CV1 line transformed by SV40 (COS-7, ATCC CRL 1651); human embryonic kidney line 293 or 293 cells subcloned for growth in suspension culture, Graham et al., J. Gen Virol., 36:59 (1977); Chinese hamster ovary cells/−DHFR [CHO, Urlaub and Chasin, Proc. Natl. Acad. Sci. USA, 77:4216 (1980); mouse sertoli cells (TM4, Mather, Biol. Reprod., 23:243-251 (1980); human lung cells (W138, ATCC CCL 75); human liver cells (Hep G2, HB 8065); and mouse mammary tumor (MMT 060562, ATCC CCL51). The selection of the appropriate host cell is deemed to be within the skill in the art.

2. Selection and Use of a Replicable Vector

The nucleic acid (e.g., cDNA or genomic DNA) encoding the desired IL-17 or LIF polypeptide or antibody antagonist may be inserted into a replicable vector for cloning (amplification of the DNA) or for expression. Various vectors are publicly available. The vector may, for example, be in the form of a plasmid, cosmid, viral particle, or phage. The appropriate nucleic acid sequence may be inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan.

The IL-17 or LIF polypeptide or antibody antagonist may be produced recombinantly not only directly, but also as a fusion polypeptide with a heterologous polypeptide, which may be a signal sequence or other polypeptide having a specific cleavage site at the N-terminus of the mature protein or polypeptide. In general, the signal sequence may be a component of the vector, or it may be a part of the DNA encoding the IL-17 or LIF polypeptide or antibody antagonist that is inserted into the vector. The signal sequence may be a prokaryotic signal sequence selected, for example, from the group of the alkaline phosphatase, penicillinase, lpp, or heat-stable enterotoxin II leaders. For yeast secretion the signal sequence may be, e.g., the yeast invertase leader, alpha factor leader (including Saccharomyces and Kluyveromyces α-factor leaders, the latter described in U.S. Pat. No. 5,010,182), or acid phosphatase leader, the C. albicans glucoamylase leader (EP 362,179 published 4 Apr. 1990), or the signal described in WO 90/13646 published 15 Nov. 1990. In mammalian cell expression, mammalian signal sequences may be used to direct secretion of the protein, such as signal sequences from secreted polypeptides of the same or related species, as well as viral secretory leaders.

Both expression and cloning vectors contain a nucleic acid sequence that enables the vector to replicate in one or more selected host cells. Such sequences are well known for a variety of bacteria, yeast, and viruses. The origin of replication from the plasmid pBR322 is suitable for most Gram-negative bacteria, the 2μ plasmid origin is suitable for yeast, and various viral origins (SV40, polyoma, adenovirus, VSV or BPV) are useful for cloning vectors in mammalian cells.

Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

An example of suitable selectable markers for mammalian cells are those that enable the identification of cells competent to take up the nucleic acid encoding the IL-17 or LIF polypeptide or antibody antagonist, such as DHFR or thymidine kinase. An appropriate host cell when wild-type DHFR is employed is the CHO cell line deficient in DHFR activity, prepared and propagated as described by Urlaub et al., Proc. Natl. Acad. Sci. USA, 77:4216 (1980). A suitable selection gene for use in yeast is the trp1 gene present in the yeast plasmid YRp7, Stinchcomb et al., Nature, 282:39 (1979); Kingsman et al., Gene, 7:141 (1979); Tschemper et al., Gene, 10:157 (1980). The trp1 gene provides a selection marker for a mutant strain of yeast lacking the ability to grow in tryptophan, for example, ATCC No. 44076 or PEP4-1 [Jones, Genetics, 85:12 (1977)].

Expression and cloning vectors usually contain a promoter operably linked to the nucleic acid encoding the IL-17 or LIF polypeptide or antibody antagonist to direct mRNA synthesis. Promoters recognized by a variety of potential host cells are well known. Promoters suitable for use with prokaryotic hosts include the β-lactamase and lactose promoter systems. Chang et al., Nature, 275:615 (1978); Goeddel et al., Nature, 281:544 (1979), alkaline phosphatase, a tryptophan (trp) promoter system, Goeddel, Nucleic Acids Res., 8:4057 (1980); EP 36,776, and hybrid promoters such as the tac promoter. deBoer et al., Proc. Natl. Acad. Sci. USA, 80:21-25 (1983). Promoters for use in bacterial systems also will contain a Shine-Dalgamo (S.D.) sequence operably linked to the DNA encoding the IL-17 or LIF polypeptide or antibody antagonist.

Examples of suitable promoting sequences for use with yeast hosts include the promoters for 3-phosphoglycerate kinase, Hitzeman et al., J. Biol. Chem., 255:2073 (1980) or other glycolytic enzymes, Hess et al., J. Adv. Enzyme Reg., 7:149 (1968); Holland, Biochemistry, 17:4900 (1978), such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657.

IL-17 or LIF polypeptide or antibody antagonist transcription from vectors in mammalian host cells is controlled, for example, by promoters obtained from the genomes of viruses such as polyoma virus, fowlpox virus (UK 2,211,504 published 5 Jul. 1989), adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, and from heat-shock promoters, provided such promoters are compatible with the host cell systems.

Transcription of a DNA encoding an IL-17 or LIF polypeptide or antibody antagonist by higher eukaryotes may be increased by inserting an enhancer sequence into the vector. Enhancers are cis-acting elements of DNA, usually about from 10 to 300 bp, that act on a promoter to increase its transcription. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. The enhancer may be spliced into the vector at a position 5′ or 3′ to the IL-17 or LIF polypeptide or antibody antagonist coding sequence but is preferably located at a site 5′ from the promoter.

Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding the IL-17 or LIF polypeptide or antibody antagonist.

Still other methods, vectors, and host cells suitable for adaptation to the synthesis of IL-17 or LIF polypeptide or antibody antagonists in recombinant vertebrate cell culture are described in Gething et al., Nature, 293:620-625 (1981); Mantei et al., Nature, 281:40-46 (1979); EP 117,060; and EP 117,058.

3. Purification of Polypeptide

Forms of IL-17 or LIF polypeptide or antibody antagonist may be recovered from culture medium or from host cell lysates. If membrane-bound, it can be released from the membrane using a suitable detergent solution (e.g. Triton®-X100) or by enzymatic cleavage. Cells employed in expression of IL-17 or LIF polypeptide or antibody antagonist can be disrupted by various physical or chemical means, such as freeze-thaw cycling, sonication, mechanical disruption, or cell lysing agents.

It may be desired to purify IL-17 or LIF polypeptide or antibody antagonist from recombinant cell proteins or polypeptides. The following procedures are exemplary of suitable purification procedures: by fractionation on an ion-exchange column; ethanol precipitation; reverse phase HPLC; chromatography on silica or on a cation-exchange resin such as DEAE; chromatofocusing; SDS-PAGE; ammonium sulfate precipitation; gel filtration using, for example, Sephadex™ G-75; protein A Sepharose columns to remove contaminants such as IgG; and metal chelating columns to bind epitope-tagged forms of the IL-17 or LIF polypeptide or antibody antagonist. Various methods of protein purification may be employed and such methods are known in the art and described for example in Deutscher, Methods in Enzymology, 182 (1990); Scopes, Protein Purification: Principles and Practice, Springer-Verlag, New York (1982). The purification step(s) selected will depend, for example, on the nature of the production process used and the particular IL-17 or LIF polypeptide or antibody antagonist produced.

E. General Uses for IL-17 or LIF Antagonists

Nucleic acid encoding the IL-17 or LIF polypeptide antagonist may also be used in gene therapy. In gene therapy applications, gene are introduced into cells in order to achieve in vivo synthesis of a therapeutically effective genetic product, for example for replacement of a defective gene. “Gene therapy” includes both conventional gene therapy where a lasting effect is achieved by a single treatment, and the administration of gene therapeutic agents, which involves the one time or repeated administration of a therapeutically effective DNA or mRNA. Antisense RNAs and DNAs can be used as therapeutic agents for blocking the expression of certain genes in vivo. It has already been shown that short antisense oligonucleotides can be imported into cells where act as inhibitors, despite their low intracellular concentrations caused by their restricted uptake by the cell membrane. Zamecnik et al., Proc. Natl. Acad. Sci. USA 83: 4143-4146 [1986]). The oligonucleotides can be modified to enhance their uptake, e.g., by substituting their negatively charged phosphodiester groups by uncharged groups.

F. Anti-IL-17, Anti-LIF Anti-IL-17R and Anti-LIFR Antibodies

The present invention further provides anti-IL-17, anti-LIF, anti-IL-17R and anti-LIFR antibodies. Exemplary antibodies include polyclonal, monoclonal, humanized, bispecific, and heteroconjugate antibodies.

1. Polyclonal Antibodies

The anti-IL-17, anti-LIF anti-IL-17R or anti-LIFR antibodies of the present invention may comprise polyclonal antibodies. Methods of preparing polyclonal antibodies are known to the skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent may include the IL-17 or LIF polypeptide, receptor or a fusion protein thereof. It may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation.

2. Monoclonal Antibodies

The anti-IL-17, anti-LIF, anti-IL-17R or anti-LIFR antibodies may, alternatively, be monoclonal antibodies. Monoclonal antibodies may be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro.

The immunizing agent will typically include the IL-17 or LIF polypeptide, receptor or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell [Goding, Monoclonal Antibodies: Principles and Practice, Academic Press, (1986) pp. 59-103]. Immortalized cell lines are usually transformed mammalian cells, particularly myeloma cells of rodent, bovine and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Rockville, Md. Human myeloma and mouse-human heteromyeloma cell lines also have been described for the production of human monoclonal antibodies. Kozbor, J. Immunol., 133:3001 (1984); Brodeur et al., Monoclonal Antibody Production Techniques and Applications, Marcel Dekker, Inc., New York, (1987) pp. 51-63.

The culture medium in which the hybridoma cells are cultured can then be assayed for the presence of monoclonal antibodies directed against an IL-17 or LIF polypeptide or receptor. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980).

After the desired hybridoma cells are identified, the clones may be subcloned by limiting dilution procedures and grown by standard methods [Goding, supra]. Suitable culture media for this purpose include, for example, Dulbecco's Modified Eagle's Medium and RPMI-1640 medium. Alternatively, the hybridoma cells may be grown in vivo as ascites in a mammal.

The monoclonal antibodies secreted by the subclones may be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies may also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA may be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences [U.S. Pat. No. 4,816,567; Morrison et al., Proc. Natl. Acad. Sci. USA 81, 6851-6855 (1984)] or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

The antibodies may be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly, Fab fragments, can be accomplished using routine techniques known in the art.

3. Humanized Antibodies

The anti-IL-17 or anti-LIF antibodies of the invention may further comprise humanized antibodies or human antibodies. Humanized forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues from a complementarity determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin. Jones et al., Nature, 321:522-525 (1986); Riechmann et al., Nature, 332:323-329 (1988); and Presta, Curr. Op. Struct. Biol., 2:593-596 (1992).

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers [Jones et al., Nature, 321:522-525 (1986); Riechmann et al., Nature, 332:323-327 (1988); Verhoeyen et al., Science, 239:1534-1536 (1988)], by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries. Hoogenboom and Winter, J. Mol. Biol., 227:381 (1991); Marks et al., J. Mol. Biol., 222:581 (1991). The techniques of Cole et al. and Boemer et al. are also available for the preparation of human monoclonal antibodies. Cole et al., Monoclonal Antibodies and Cancer Therapy, Alan R. Liss, p. 77 (1985) and Boemer et al., J. Immunol., 147(1):86-95 (1991). Similarly, human antibodies can be made by introducing human immunoglobulin loci into transgenic animals, e.g., mice in which the endogenous immunoglobulin genes have been partially or complete inactivated. Upon challenge, human antibody production is observed, which closely resembles that seen in humans in all respects, including gene rearrangement, assembly and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10: 779-783 (1992); Lonberg et al., Nature 368: 856-859 (1994); Morrison, Nature 368: 812-13 (1994); Fishwild et al., Nature Biotechnology 14: 845-51 (1996); Neuberger, Nature Biotechnology 14: 826 (1996); Lonberg and Huszar, Intern. Rev. Immunol. 13: 65-93 (1995).

4. Antibody Dependent Enzyme Mediated Prodrug Therapy (ADEPT)

The antibodies of the present invention may also be used in ADEPT by conjugating the antibody to a prodrug-activating enzyme which converts a prodrug (e.g. a peptidyl chemotherapeutic agent, see WO 81/01145) to an active anti-cancer drug. See, for example, WO 88/07378 and U.S. Pat. No. 4,975,278.

The enzyme component of the immunoconjugate useful for ADEPT includes any enzyme capable of acting on a prodrug in such as way so as to convert it into its more active, cytotoxic form.

Enzymes that are useful in the method of this invention include, but are not limited to, glycosidase, glucose oxidase, human lysozyme, human glucuronidase, alkaline phosphatase useful for converting phosphate-containing prodrugs into free drugs; arylsulfatase useful for converting sulfate-containing prodrugs into free drugs; cytosine deaminase useful for converting non-toxic 5-fluorocytosine into the anti-cancer drug 5-fluorouracil; proteases, such as serratia protease, thermolysin, subtilisin, carboxypeptidases (e.g., carboxypeptidase G2 and carboxypeptidase A) and cathepsins (such as cathepsins B and L), that are useful for converting peptide-containing prodrugs into free drugs; D-alanylcarboxypeptidases, useful for converting prodrugs that contain D-amino acid substituents; carbohydrate-cleaving enzymes such as β-galactosidase and neuraminidase useful for converting glycosylated prodrugs into free drugs; β-lactamase useful for converting drugs derivatized with β-lactams into free drugs; and penicillin amidases, such as penicillin Vamidase or penicillin G amidase, useful for converting drugs derivatized at their amine nitrogens with phenoxyacetyl or phenylacetyl groups, respectively, into free drugs. Alternatively, antibodies with enzymatic activity, also known in the art as “abzymes” can be used to convert the prodrugs of the invention into free active drugs (see, e.g., Massey, Nature 328: 457-458 (1987)). Antibody-abzyme conjugates can be prepared as described herein for delivery of the abzyme to a tumor cell population.

The enzymes of this invention can be covalently bound to the anti-IL-17 or anti-LIF antibodies by techniques well known in the art such as the use of the heterobifunctional cross-linking agents discussed above. Alternatively, fusion proteins comprising at least the antigen binding region of the antibody of the invention linked to at least a functionally active portion of an enzyme of the invention can be constructed using recombinant DNA techniques well known in the art (see, e.g. Neuberger et al., Nature 312: 604-608 (1984)).

5. Bispecific Antibodies

Bispecific antibodies are monoclonal, preferably human or humanized, antibodies that have binding specificities for at least two different antigens. In the present case, one of the binding specificities is for an IL-17 or an LIF polypeptide or receptor, the other one is for any other antigen, and preferably for a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities. Milstein and Cuello, Nature, 305:537-539 (1983). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229: 81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Fab′ fragments may be directly recovered from E. coli and chemically coupled to form bispecific antibodies. Shalaby et al., J. Exp. Med. 175: 217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers, Kostelny et al., J. Immunol. 148(5): 1547-1553 (1992), wherein the leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90: 6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152: 5368 (1994).

Antibodies with more than two valencies are contemplated. For example, trispecific antibodies can be prepared. Tutt et al., J. Immunol. 147: 60 (1991).

Exemplary bispecific antibodies may bind to two different epitopes on a given IL-17, LIF polypeptide or receptor. Alternatively, an anti-IL17 or LIF arm may be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular IL-17, LIF polypeptide or receptor. Bispecific antibodies may also be used to localize cytotoxic agents to cells which express a particular IL-17, LIF polypeptide or receptor. These antibodies possess an IL-17, LIF polypeptide or receptor-binding arm and an arm which binds a cytotoxic agent or a radionuclide chelator, such as EOTUBE, DPTA, DOTA, or TETA. Another bispecific antibody of interest may bind IL-17 or LIF and further binds tissue factor (TF).

6. Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells, U.S. Pat. No. 4,676,980, and for treatment of HIV infection (WO 91/00360; WO 92/200373; EP 03089). It is contemplated that the antibodies may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

7. Effector Function Engineering

It may be desirable to modify the antibody of the invention with respect to effector function, so as to enhance the effectiveness of the antibody. For example cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med. 176:1191-1195 (1992) and Shopes, B. J. Immunol. 148:2918-2922 (1992). Homodimeric antibodies with enhanced anti-tumor activity may also be prepared using heterobifunctional cross-linkers as described in Wolff et al. Cancer Research 53:2560-2565 (1993). Alternatively, an antibody can be engineered which has dual Fc regions and may thereby have enhanced complement lysis and ADCC capabilities. See Stevenson et al., Anti-Cancer Drug Design 3: 219-230 (1989).

8. Immunoconjugates

The invention also pertains to immunoconjugates comprising an antibody conjugated to a cytotoxic agent such as a chemotherapeutic agent, toxin (e.g. an enzymatically active toxin of bacterial, fungal, plant or animal origin, or fragments thereof, or a small molecule toxin), or a radioactive isotope (i.e., a radioconjugate).

Chemotherapeutic agents useful in the generation of such immunoconjugates have been described above. Enzymatically active protein toxins and fragments thereof which can be used include diphtheria A chain, nonbinding active fragments of diphtheria toxin, cholera toxin, botulinus toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, saporin, mitogellin, restrictocin, phenomycin, enomycin and the tricothecenes. Small molecule toxins include, for example, calicheamicins, maytansinoids, palytoxin and CC1065. A variety of radionuclides are available for the production of radioconjugated antibodies. Examples include ²¹²Bi, ¹³¹I, ¹³¹In, ⁹⁰Y and ¹⁸⁶Re.

Conjugates of the antibody and cytotoxic agent are made using a variety of bifunctional protein coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutaraldehyde), bis-azido compounds (such as bis(p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody may be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g. avidin) which is conjugated to a cytotoxic agent (e.g. a radionucleotide).

9. Immunoliposomes

The antibodies disclosed herein may also be formulated as immunoliposomes. Liposomes containing the antibody are prepared by methods known in the art, such as described in Epstein et al., Proc. Natl. Acad. Sci. USA 82:3688 (1985); Hwang et al., Proc. Natl. Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem. 257: 286-288 (1982) via a disulfide interchange reaction. A chemotherapeutic agent (such as Doxorubicin) is optionally contained within the liposome. See Gabizon et al., J. National Cancer Inst. 81(19): 1484 (1989).

H. IL-17 and LIF Antagonists

This invention is also directed to methods of screening compounds to identity those that prevent from occurring a biological effect of an IL-17 or LIF polypeptide (antagonists). Screening assays for antagonist drug candidates are designed to identity compounds that bind or complex with the IL-17 or LIF polypeptides or receptor, or otherwise interfere with the interaction of these polypeptides with other cellular proteins. Such screening assays will include assays amenable to high-throughput screening of chemical libraries, making them particularly suitable for identifying small molecule drug candidates.

The assays can be performed in a variety of formats, including protein-protein binding assays, biochemical screening assays, immunoassays, and cell-based assays, which are well characterized in the art. For example, to screen for antagonists and/or agonists of IL-17 or LIF signaling, the assay mixture can be incubated under conditions whereby, but for the presence of the candidate pharmacological agent, IL-17 induces TNF-α release from THP-1 cells with a reference activity. Alternatively, the tested activity can be the release of nitric oxide (NO) and proteoglycans from cartilage in the presence of IL-17 and/or LIF in combination with or in the absence of IL-1α treatment.

In binding assays, the interaction is binding and the complex formed can be isolated or detected in the reaction mixture. In a particular embodiment, the IL-17 or LIF polypeptide or receptor or the drug candidate is immobilized on a solid phase, e.g., on a microtiter plate, by covalent or non-covalent attachments. Non-covalent attachment generally is accomplished by coating the solid surface with a solution of the IL-17 or LIF polypeptide or receptor and drying. Alternatively, an immobilized antibody, e.g., a monoclonal antibody, specific for the IL-17 or LIF polypeptide or receptor to be immobilized can be used to anchor it to solid surface. The assay is performed by adding the non-immobilized component, which may be labeled by a detectable label, to the immobilized component, which may be labeled by a detectable label, to the immobilized component, e.g., the coated surface containing the anchored component. When the reaction is complete, the non-reacted components are removed, e.g., by washing, and complexes anchored on the solid surface are detected. When the originally non-immobilized component carries a detectable label, the detection of label immobilized on the surface indicates that complexing occurred. Where the originally non-immobilized component does not carry a label, complexing can be detected, for example, by using a labeled antibody specifically binding the immobilized complex.

If the candidate compound interacts with but does not bind to a particular IL-17 or LIF polypeptide, receptor or antibody, its interaction with that polypeptide or antibody can be assayed by methods well known for detecting protein-protein interactions. Such assays include traditional approaches, such as, e.g., cross-linking, co-immunoprecipitation, and co-purification through gradients or chromatographic columns. In addition, protein-protein interactions can be monitored through gradients or chromatographic columns. In addition, protein-protein interactions can be monitored by using a yeast-based genetic system described by Fields and co-workers (Fields and Song, Nature 340: 245-246 (1989); Chien et al., Proc. Natl. Acad. Sci. USA 88: 9578-9582 (1991) as disclosed by Chevray and Nathans, Proc. Natl. Acad. Sci. USA 89: 5789-5791 (1991). Many transcriptional activators, such as yeast GAL4, consist of two physically discrete modular domains, one acting as the DNA-binding domain, while the other functions as the transcription-activation domain. The yeast expression system described in the foregoing publications (generally referred to as the “two-hybrid system”) takes advantage of this property, and employs two hybrid proteins, one in which the target protein is fused to the DNA-binding domain of GAL4, and another, in which candidate activating proteins are fused to the activation domain. The expression of GAL1-lacZ reporter gene under control of a GAL4-activated promoter depends on reconstitution of GAL4 activity via protein-protein interaction. Colonies containing interacting polypeptide are detected with chromogenic substrate for β-galactosidase. A complete kit (MATCHMAKER™) for identifying protein-protein interactions between two specific proteins using the two-hybrid technique is commercially available from Clontech. This system can also be extended to map protein domains involved in specific protein interactions as well as to pinpoint amino acid residues that are crucial for these interactions.

Compounds that interfere with the interaction of (1) an IL-17, LIF polypeptide or receptor and (2) other intra- or extracellular components can be tested as follows: usually a reaction mixture is prepared containing the IL-17, LIF polypeptide or receptor and the intra- or extracellular component under conditions and for a time allowing for the interaction and binding of (1) and (2). To test the ability of a candidate compound to inhibit binding, the reaction is run in the absence and in the presence of the test compound. In addition, a placebo may be added to a third reaction mixture, to serve as a positive control. The binding (complex formation) between the test compound and the intra- or extracellular component present in the mixture is monitored as described hereinabove. The formation of a complex in the control reaction(s) but not in the reaction mixture containing the test compound indicates that the test compound interferes with the interaction of the test compound and its reaction partner.

Antagonists may be detected by combining the IL-17 or LIF polypeptide or receptor and a potential antagonist with membrane-bound IL-17 or LIF polypeptide receptors or recombinant receptors under appropriate conditions for a competitive inhibition assay. The IL-17 or LIF polypeptide or receptor can be labeled, such as by radioactivity, such that the number of IL-17 or LIF polypeptide molecules bound to the receptors can be used to determine the effectiveness of the potential antagonist. The gene encoding the receptor can be identified by numerous methods known to those of skill in the art, for example, ligand panning and FACS sorting. Coligan et al., Current Protocols in Immun. 1(2): Ch. 5 (1991). Preferably, expression cloning is employed wherein polyadenylated RNA is prepared from a cell responsive to the IL-17 or LIF polypeptide or receptor and a cDNA library created from this RNA is divided into pools and used to transfect COS cells or other cells that are not responsive to the IL-17 or LIF polypeptide or receptor. Transfected cells that are grown on glass slides are exposed to labeled IL-17 or LIF polypeptide or receptor. The IL-17 or LIF polypeptide or receptor can be labeled by a variety of means including iodination or inclusion of a recognition site for a site-specific protein kinase. Following fixation and incubation, the slides are subjected to autoradiographic analysis. Positive pools are identified and sub-pools are prepared and re-transfected using an interactive sub-pooling and re-screening process, eventually yielding a single clone that encodes the putative receptor.

As an alternative approach for receptor identification, labeled IL-17 or LIF polypeptide or receptor can be photoaffinity-linked with cell membrane or extract preparations that express the receptor molecule. Cross-linked material is resolved by PAGE and exposed to X-ray film. The labeled complex containing containing the receptor can be excised, resolved into peptide fragments, and subjected to protein micro-sequencing. The amino acid sequence obtained from micro-sequencing would be used to design a set of degenerate oligonucleotide probes to screen a cDNA library to identity the gene encoding the putative receptor.

In another assay for antagonists, mammalian cells or a membrane preparation expressing the receptor would be incubated with labeled IL-17 or LIF polypeptide or antibody in the presence of the candidate compound. The ability of the compound to enhance or block this interaction could then be removed.

More specific examples of potential antagonists include an oligonucleotide that binds to the fusions of immunoglobulin with IL-17 or LIF polypeptide or receptor, and, in particular, antibodies including, without limitation, poly- and monoclonal antibodies and antibody fragments, single-chain antibodies, anti-idiotypic antibodies, and chimeric or humanized versions of such antibodies or fragments, as well as human antibodies and antibody fragments. Alternatively, a potential antagonist may be a closely related protein, for example, a mutated form of the IL-17 or LIF polypeptide or receptor that recognizes the receptor but imparts no effect, thereby competitively inhibiting the action of IL-17 or LIF polypeptide.

Another potential IL-17 or LIF antagonist is an antisense RNA or DNA construct prepared using antisense technology, where, e.g., an antisense RNA or DNA molecule acts to block directly the translation of mRNA by hybridizing to targeted mRNA and preventing its translation into protein. Antisense technology can be used to control gene expression through triple-helix formation or antisense DNA or RNA, both of which methods are based on binding of a polynucleotide to DNA or RNA. For example, the 5′ coding portion of the polynucleotide sequence, which encodes the mature IL-17 or LIF polypeptide, is used to design an antisense RNA oligonucleotide sequence, which encodes a mature IL-17 or LIF polypeptide, respectively, is used to design an antisense RNA oligonucleotide of about 10 to 40 base pairs in length. A DNA oligonucleotide is designed to be complementary to a region of the gene involved in transcription (triple helix—see Lee et al., Nucl. Acids Res. 6: 3073 (1979); Cooney et al., Science 241: 456 (1988); Dervan et al., Science 251: 1360 (1991)), thereby preventing transcription and the production of the IL-17 or LIF polypeptide. The antisense RNA oligonucleotide hybridizes to the mRNA in vivo and blocks translation of the mRNA molecule into the IL-17 or LIF polypeptide (antisense—Okano, Neurochem. 546: 560 (1991); Oligodeoxynucleotides as Antisense Inhibitors of Gene Expression (CRC Press: Boca Raton, Fla., 1988). The oligonucleotides described above can also be delivered to cells such that the antisense RNA or DNA may be expressed in vivo to inhibit production of the IL-17 or LIF polypeptide. When antisense DNA is used, oligodeoxyribonucleotides derived from the translation-initiation site, e.g., between about −10 and +10 positions of the target gene nucleotide sequence, are preferred.

Potential antagonists include small molecules that bind to the active site, the receptor binding site, or growth factor or other relevant binding site of the IL-17 or LIF polypeptide, thereby blocking the normal biological activity of the IL-17 or LIF polypeptide. Examples of small molecules include, but are not limited to, small peptides or peptide-like molecules, preferably soluble peptides, and synthetic non-peptidyl organic or inorganic compounds.

Ribozymes are enzymatic RNA molecules capable of catalyzing the specific cleavage of RNA. Ribozymes act by sequence-specific hybridization to the complementary target RNA, followed by endonucleic cleavage. Specific ribozyme cleavage sites within a potential RNA target can be identified by known techniques. For further details, see e.g., Rossi, Current Biology 4: 469-471 (1994) and PCT publication No. WO 97/33551 (published Sep. 18, 1997).

Nucleic acid molecules in triple-helix formation used to inhibit transcription should be single-stranded and composed of deoxynucleotides. The base composition of these oligonucleotides is designed such that it promotes triple-helix formation via Hoogsteen base-pairing rules, which generally require sizeable stretches of purines or pyrimidines on one strand of a duplex. For further details see, e.g., PCR publication No. WO 97/33551, supra.

I. Pharmaceutical Compositions and Dosages

The IL-17 and LIF antagonists usable with the method of the invention can be adminstered for the treatment of cartilagenous disorders in the form of a pharmaceutical composition. Additionally, lipofections or liposomes can be used to deliver the IL-17 or LIF antagonist.

Where antibody fragments are used, the smallest inhibitory fragment which specifically binds to the binding domain of the target protein is preferred. For example, based upon the variable region sequences of an antibody, peptide molecules can be designed which retain the ability to bind the target protein sequence. Such peptides can be synthesized chemically and/or produced by recombinant DNA technology (see, e.g. Marasco et al., Proc. Natl. Acad. Sci. USA 90: 7889-7893 [1993]).

Therapeutic formulations are prepared for storage by mixing the active ingredient having the desired degree of purity with optional pharmaceutically acceptable carriers, excipients or stabilizers (Remington's Pharmaceutical Sciences 16th edition, Osol, A. Ed. [1980]), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

In order for the formulations to be used for in vivo administration, they must be sterile. The formulation may be rendered sterile by filtration through sterile filtration membranes, prior to or following lyophilization and reconstitution. The therapeutic compositions herein generally are placed into a container having a sterile access port, for example, an intravenous solution bag or vial having a stoopper pierceable by a hypodermic injection needle.

The formulation herein may also contain more than one active compound as necessary for the particular indication being treated, preferably those with complementary activities that do not adversely affect each other. Alternatively, or in addition, the composition may comprise a cytotoxic agent, cytokine or growth inhibitory agent. Such molecules are suitably present in combination in amounts that are effective for the purpose intended.

The route of administration is in accordance with known and accepted methods, e.g., injection or infusion by intravenous, intraperitoneal, intramuscular, intraarterial, intralesional or intraarticular routes, topical administration, by sustained release or extended-release means. Optionally, the active compound or formulation is injected directly into the afflicted cartilagenous region or articular joint.

The active agents of the present invention, e.g. antibodies, are administered to a mammal, preferably a human, in accord with known methods, such as intravenous administration as a bolus or by continuous infusion over a period of time, by intramuscular, intraperitoneal, intracerebral, intracerobrospinal, subcutaneous, intra-articular, intrasynovial, intrathecal, intraoccular, intralesional, oral, topical, inhalation or through sustained release.

Dosages and desired drug concentration of pharmaceutical compositions of the present invention may vary depending on the particular use envisioned. The determination of the appropriate dosage or route of administration is well within the skill of an ordinary artisan. Animal experiments provide reliable guidance for the determination of effective doses for human therapy. Interspecies scaling of effective doses can be performed following the principles laid down by Mordenti, J. and Chappell, W. “The Use of Interspecies Scaling in Toxicokinetics,” In Toxicokinetics and New Drug Development, Yacobi et al., Eds, Pergamon Press, New York 1989, pp. 42-46.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ-ethyl-L-glutamate, non-degradable ethylene-vinyl acetate, degradable lacetic acid-glycolic acid copolymers such as the LUPRON DEPOT™ (injectable microspheres composed of lacetic acid-glycolic acid copolymer and leuprolide acetate), and poly-D-(−)-3-hydroxybutyric acid. Microencapsulation of recombinant proteins for sustained release has been successfully performed with human growth hormone (rhGH), interferon-(rhIFN-), interleukin-2, and MN rpg 120. Johnson et al., Nat. Med. 2: 795-799 (1996); Yasuda et al., Biomed. Ther. 27: 1221-1223 (1993); Hora et al., Bio/Technology 8: 755-758 (1990); Cleland, “Design and Production of Single Immunization Vaccines Using Polylactide Polyglycolide Microsphere Systems,” in Vaccine Design The Subunit and Adjuvant Approach, Powell and Newman, eds., (Penum Press: New York, 1995), pp. 439-462; WO 97/03692; WO 96/40072; WO 96/07399; and U.S. Pat. No. 5,654,010.

The sustained-release formulations of these proteins may be developed using poly lacetic-coglycolic acid (PLGA) polymer due to its biocompatibility and wide range of biodegradable properties. The degradation products of PLGA, lacetic and glycolic acids, can be cleared quickly within the human body. Moreover, the degradability of this polymer can be adjusted from months to years depending on its molecular weight and composition. Lewis, “Controlled release of bioactive agents from lactide/glycolide polymer”, in Biodegradable Polymers as Drug Delivery Systems (Marcel Dekker; New York, 1990), M. Chasin and R. Langer (Eds.) pp. 1-41.

While polymers such as ethylene-vinyl acetate and lacetic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

When in vivo administration of the IL-17 or LIF antagonists are used, normal dosage amounts may vary from about 10 ng/kg up to about 100 mg/kg of mammal body weight or more per day, preferably about 1 mg/kg/day to 10 mg/kg/day, depending upon the route of administration. Guidance as to particular dosages and methods of delivery is provided in the literature; see, for example, U.S. Pat. No. 4,657,760; 5,206,344; or 5,225,212. It is within the scope of the invention that different formulations will be effective for different treatments and different disorders, and that administration intended to treat a specific organ or tissue may necessitate delivery in a manner different from that to another organ or tissue. Moreover, dosages may be administered by one or more separate administrations, or by continuous infusion. For repeated administrations over several days or longer, depending on the condition, the treatment is sustained until a desired suppression of disease symptoms occurs. However, other dosage regimens may be useful. The progress of this therapy is easily monitored by conventional techniques and assays.

K. Methods of Treatment

For the prevention or treatment of disease, the appropriate dosage of an active agent, will depend on the type of disease to be treated, as defined above, the severity and course of the disease, whether the agent is administered for preventive or therapeutic purposes, previous therapy, the patient's clinical history and response to the agent, and the discretion of the attending physician. The agent is suitably administered to the patient at one time or over a series of treatments.

It is contemplated that the polypeptides, antibodies and other active compounds of the present invention may be used to treat various cartilagenous disorders. Exemplary conditions or disorders to be treated with the polypeptides of the invention, include, but are not limited to systemic lupus erythematosis, rheumatoid arthritis, juvenile chronic arthritis, osteoarthritis, spondyloarthropathies, systemic sclerosis (scleroderma), idiopathic inflammatory myopathies (dermatomyositis, polymyositis), Sjögren's syndrome, systemic vasculitis, sarcoidosis, autoimmune hemolytic anemia (immune pancytopenia, paroxysmal nocturnal hemoglobinuria), autoimmune thrombocytopenia (idiopathic thrombocytopenic purpura, immune-mediated thrombocytopenia), thyroiditis (Grave's disease, Hashimoto's thyroiditis, juvenile lymphocytic thyroiditis, atrophic thyroiditis), diabetes mellitus, immune-mediated renal disease (glomerulonephritis, tubulointerstitial nephritis), demyelinating diseases of the central and peripheral nervous systems such as multiple sclerosis, idiopathic demyelinating polyneuropathy or Guillain-Barré syndrome, and chronic inflammatory demyelinating polyneuropathy, hepatobiliary diseases such as infectious hepatitis (hepatitis A, B, C, D, E and other non-hepatotropic viruses), autoimmune chronic active hepatitis, primary biliary cirrhosis, granulomatous hepatitis, and sclerosing cholangitis, inflammatory bowel disease (ulcerative colitis: Crohn's disease), gluten-sensitive enteropathy, and Whipple's disease, autoimmune or immune-mediated skin diseases including bullous skin diseases, erythema multiforme and contact dermatitis, psoriasis, allergic diseases such as asthma, allergic rhinitis, atopic dermatitis, food hypersensitivity and urticaria, immunologic diseases of the lung such as eosinophilic pneumonias, idiopathic pulmonary fibrosis and hypersensitivity pneumonitis, transplantation associated diseases including graft rejection and graft-versus-host-disease.

In systemic lupus erythematosus, the central mediator of disease is the production of auto-reactive antibodies to self proteins/tissues and the subsequent generation of immune-mediated inflammation. These antibodies either directly or indirectly mediate tissue injury. Although T lymphocytes have not been shown to be directly involved in tissue damage, T lymphocytes are required for the development of auto-reactive antibodies. The genesis of the disease is thus T lymphocyte dependent. Multiple organs and systems are affected clinically including kidney, lung, musculoskeletal system, mucocutaneous, eye, central nervous system, cardiovascular system, gastrointestinal tract, bone marrow and blood.

Rheumatoid arthritis (RA) is a chronic systemic autoimmune inflammatory disease that affects the synovial membrane of multiple joints and which results in injury to the articular cartilage. The pathogenesis is T lymphocyte dependent and is associated with the production of rheumatoid factors, auto-antibodies directed against endogenous proteins, with the resultant formation of immune complexes that attain high levels in joint fluid and blood. These complexes may induce infiltration by lymphocytes, monocytes, and neutrophils into the synovial compartment. Tissues affected are primarily the joints, often in symmetrical pattern. However, disease outside the joints occurs in two major forms. In one form, typical lesions are pulmonary fibrosis, vasculitis, and cutaneous ulcers. The second form is the so-called Felty's syndrome which occurs late in the RA disease course, sometimes after joint disease has become quiescent, and involves the presence of neutropenia, thrombocytopenia and splenomegaly. This can be accompanied by vasculitis in multiple organs and occurrence of infarcts, skin ulcers and gangrene. Patients often also develop rheumatoid nodules in the subcutis tissue overlying affected joints; in late stages, the nodules have necrotic centers surrounded by a mixed inflammatory cellular infiltrate. Other manifestations which can occur in RA include: pericarditis, pleuritis, coronary arteritis, intestitial pneumonitis with pulmonary fibrosis, keratoconjunctivitis sicca, and rheumatoid nodules.

Juvenile chronic arthritis is a chronic idiopathic inflammatory disease which begins often at less than 16 years of age and which has some similarities to RA. Some patients which are rheumatoid factor positive are classified as juvenile rheumatoid arthritis. The disease is sub-classified into three major categories: pauciarticular, polyarticular, and systemic. The arthritis can be severe and leads to joint ankylosis and retarded growth. Other manifestations can include chronic anterior uveitis and systemic amyloidosis.

Spondyloarthropathies are a group of disorders with some common clinical features and the common association with the expression of HLA-B27 gene product. The disorders include: ankylosing sponylitis, Reiter's syndrome (reactive arthritis), arthritis associated with inflammatory bowel disease, spondylitis associated with psoriasis, juvenile onset spondyloarthropathy and undifferentiated spondyloarthropathy. Distinguishing features include sacroileitis with or without spondylitis; inflammatory asymmetric arthritis; association with HLA-B27 (a serologically defined allele of the HLA-B locus of class I MHC); ocular inflammation, and absence of autoantibodies associated with other rheumatoid disease. The cell most implicated as key to induction of the disease is the CD8+ T lymphocyte, a cell which targets antigen presented by class I MHC molecules. CD8+ T cells may react against the class I MHC allele HLA-B27 as if it were a foreign peptide expressed by MHC class I molecules. It has been hypothesized that an epitope of HLA-B27 may mimic a bacterial or other microbial antigenic epitope and thus induce a CD8+ T cells response.

Systemic sclerosis (scleroderma) has an unknown etiology. A hallmark of the disease is induration of the skin which is likely induced by an active inflammatory process. Scleroderma can be localized or systemic. Vascular lesions are common, and endothelial cell injury in the microvasculature is an early and important event in the development of systemic sclerosis. An immunologic basis is implied by the presence of mononuclear cell infiltrates in the cutaneous lesions and the presence of anti-nuclear antibodies in many patients. ICAM-1 is often upregulated on the cell surface of fibroblasts in skin lesions suggesting that T cell interaction with these cells may have a role in the pathogenesis of the disease. Other organs may also be involved. In the gastrointestinal tract, smooth muscle atrophy and fibrosis can result in abnormal peristalsis/motility. In the kidney, concentric subendothelial intimal proliferation affecting small arcuate and interlobular arteries can result in reduced renal cortical blood flow and thus proteinuria, azotemia and hypertension. In skeletal and cardiac muscle, atrophy, interstitial fibrosis/scarring, and necrosis can occur. Finally, the lung can have interstitial pneumonitis and interstitial fibrosis.

Idiopathic inflammatory myopathies including dermatomyositis, polymyositis and others are disorders of chronic muscle inflammation of unknown etiology resulting in muscle weakness. Muscle injury/inflammation is often symmetric and progressive. Autoantibodies are associated with most forms. These myositis-specific autoantibodies are directed against and inhibit the function of components involved in protein synthesis.

Sjögren's syndrome is the result of immune-mediated inflammation and subsequent functional destruction of the tear glands and salivary glands. The disease can be associated with or accompanied by inflammatory connective tissue diseases. The disease is associated with autoantibody production against Ro and La antigens, both of which are small RNA-protein complexes. Lesions result in keratoconjunctivitis sicca, xerostomia, with other manifestations or associations including bilary cirrhosis, peripheral or sensory neuropathy, and palpable purpura.

Systemic vasculitis are diseases in which the primary lesion is inflammation and subsequent damage to blood vessels which results in ischemia/necrosis/degeneration to tissues supplied by the affected vessels and eventual end-organ dysfunction in some cases. Vasculitides can also occur as a secondary lesion or sequelae to other immune-inflammatory mediated diseases such as rheumatoid arthritis, systemic sclerosis, etc, particularly in diseases also associated with the formation of immune complexes. Diseases in the primary systemic vasculitis group include: systemic necrotizing vasculitis: polyarteritis nodosa, allergic angiitis and granulomatosis, polyangiitis; Wegener's granulomatosis; lymphomatoid granulomatosis; and giant cell arteritis. Miscellaneous vasculitides include: mucocutaneous lymph node syndrome (MLNS or Kawasaki's disease), isolated CNS vasculitis, Behet's disease, thromboangiitis obliterans (Buerger's disease) and cutaneous necrotizing venulitis. The pathogenic mechanism of most of the types of vasculitis listed is believed to be primarily due to the deposition of immunoglobulin complexes in the vessel wall and subsequent induction of an inflammatory response either via ADCC, complement activation, or both.

Sarcoidosis is a condition of unknown etiology which is characterized by the presence of epithelioid granulomas in nearly any tissue in the body; involvement of the lung is most common. The pathogenesis involves the persistence of activated macrophages and lymphoid cells at sites of the disease with subsequent chronic sequelae resultant from the release of locally and systemically active products released by these cell types.

Autoimmune hemolytic anemia including autoimmune hemolytic anemia, immune pancytopenia, and paroxysmal noctural hemoglobinuria is a result of production of antibodies that react with antigens expressed on the surface of red blood cells (and in some cases other blood cells including platelets as well) and is a reflection of the removal of those antibody coated cells via complement mediated lysis and/or ADCC/Fc-receptor-mediated mechanisms.

In autoimmune thrombocytopenia including thrombocytopenic purpura, and immune-mediated thrombocytopenia in other clinical settings, platelet destruction/removal occurs as a result of either antibody or complement attaching to platelets and subsequent removal by complement lysis, ADCC or FC-receptor mediated mechanisms.

Thyroiditis including Grave's disease, Hashimoto's thyroiditis, juvenile lymphocytic thyroiditis, and atrophic thyroiditis, are the result of an autoimmune response against thyroid antigens with production of antibodies that react with proteins present in and often specific for the thyroid gland. Experimental models exist including spontaneous models: rats (BUF and BB rats) and chickens (obese chicken strain); inducible models: immunization of animals with either thyroglobulin, thyroid microsomal antigen (thyroid peroxidase).

Diabetes mellitus is a genetic disorder of metabolism of carbohydrate, protein and fat associated with a relative or absolute insufficiency of insulin secretion and with various degrees of insulin resistance. In its fully developed clinical expression, it is characterized by fasting hyperglycemia and in the majority of long-standing patients by atherosclerotic and microangiopathic vascular disease and neuropathy. Differences between various forms of the disease are expressed in terms of cause and pathogenesis, natural history, and response to treatment. Thus, diabetes is not a single disease but a syndrome.

Type I, or insulin-dependent diabetes mellitus (IDDM) occurs in approximately 10 percent of all diabetic patients in the Western world. Type I diabetes mellitus or insulin-dependent diabetes is the autoimmune destruction of pancreatic islet β-cells; this destruction is mediated by auto-antibodies and auto-reactive T cells. Antibodies to insulin or the insulin receptor can also produce the phenotype of insulin-non-responsiveness.

Classically, this type of disease occurs most commonly in childhood and adolescence; however, it can be recognized and become symptomatic at any age. In the most common type of IDDM (Type IA), it has been postulated that environmental (acquired) factors such as certain viral infections, and possibly chemical agents, superimposed on genetic factors, may lead to cell-mediated autoimmune destruction of β cells. Thus, genetically determined abnormal immune responses (linked to HLA associations) characterized by cell mediated and humoral autoimmunity are thought to play a pathogenetic role after evocation by an environmental factor. A second type of IDDM (Type IB) is believed to be due to primary autoimmunity. These patients have associated autoimmune endocrine diseases such as Hashimoto's thyroiditis, Graves' disease, Addison's disease, primary gonadal failure, and associated nonendocrine autoimmune diseases such as pernicious anemia, connective tissue diseases, celiac disease and myasthenia gravis. Insulin dependency implies that administration of insulin is essential to prevent spontaneous ketosis, coma, and death. However, even with insulin treatment, diabetic patients can still have many of the additional problems associated with diabetes, i.e. connective tissue disorders, neuropathy, etc.

The second type of diabetes, Type II or non-insulin-dependent diabetes mellitus (NIDDM), present in approximately 90% of all diabetics, also has a genetic basis. Patients with type II diabetes may have a body weight that ranges from normal to excessive. Obesity and pathological insulin resistance are by no means essential in the evolution of NIDDM. In the majority of patients with NIDDM, a diagnosis is made in middle age. Patients with NIDDM are non-insulin-dependent for prevention of ketosis, but they may require insulin for correction of symptomatic or nonsymptomatic persistent fasting hyperglycemia if this cannot bye achieved with the use of diet or oral agents. Thus, therapeutic administration of insulin does not distinguish between IDDM and NIDDM. In some NIDDM families, the insulin secretory responses to glucose are so low that they may resemble those of early Type I diabetes at any point in time. Early in its natural history, the insulin secretory defect and insulin resistance may be reversible by treatment (i.e. weight reduction) with normalization of glucose tolerance. The typical chronic complications of diabetes, namely macroangiopathy, microangiopathy, neuropathy, and cataracts seen in IDDM are seen in NIDDM as well.

Other types of diabetes include entities secondary to or associated with certain other conditions or syndromes. Diabetes may be secondary to pancreatic disease or removal of pancreatic tissue; endocrine diseases such as acromegaly, Cushing's syndrome, pheochromocytoma, glucagonoma, somatostatinoma, or primary aldosteronism; the administration of hormones, causing hyperglycemia; and the administration of certain drugs (i.e. antihypertensive drugs, thiazide diuretics, preparations containing estrogen, psychoactive drugs, sympathomimetic agents). Diabetes may be associated with a large number of genetic syndromes. Finally, diabetes may be associated with genetic defects of the insulin receptor or due to antibodies to the insulin receptor with or without associated immune disorders.

Immune mediated renal diseases, including glomerulonephritis and tubulointerstitial nephritis, are the result of antibody or T lymphocyte mediated injury to renal tissue either directly as a result of the production of autoreactive antibodies or T cells against renal antigens or indirectly as a result of the deposition of antibodies and/or immune complexes in the kidney that are reactive against other, non-renal antigens. Thus, other immune-mediated diseases that result in the formation of immune-complexes can also induce immune mediated renal disease as an indirect sequelae. Both direct and indirect immune mechanisms result in inflammatory response that produces/induces lesion development in renal tissues with resultant organ function impairment and in some cases progression to renal failure. Both humoral and cellular immune mechanisms can be involved in the pathogenesis of lesions. Demyelinating diseases of the central and peripheral nervous systems, including multiple sclerosis; idiopathic demyelinating polyneuropathy or Guillain-Barré syndrome; and Chronic Inflammatory Demyelinating Polyneuropathy, are believed to have an autoimmune basis and result in nerve demyelination as a result of damage caused to oligodendrocytes or to myelin directly. In MS there is evidence to suggest that disease induction and progression is dependent on T lymphocytes. Multiple sclerosis is a demyelinating disease that is T lymphocyte-dependent and has either a relapsing-remitting course or a chronic progressive course. The etiology is unknown; however, viral infections, genetic predisposition, environment, and autoimmunity all contribute. Lesions contain infiltrates of predominantly T lymphocyte mediated, microglial cells and infiltrating macrophages; CD4+T lymphocytes are the predominant cell type at lesions. The mechanism of oligodendrocyte cell death and subsequent demyelination is not known but is likely T lymphocyte driven.

Inflammatory and fibrotic lung disease, including eosinophilic pneumonias, idiopathic pulmonary fibrosis, and hypersensitivity pneumonitis may involve a disregulated immune-inflammatory response.

Inhibition of that response would be of therapeutic benefit and within the scope of the invention.

Autoimmune or immune-mediated skin disease, including bullous skin diseases, erythema multiforme, and contact dermatitis are mediated by auto-antibodies, the genesis of which is T lymphocyte-dependent.

Psoriasis is a T lymphocyte-mediated inflammatory disease. Lesions contain infiltrates of T lymphocytes, macrophages and antigen processing cells, and some neutrophils.

Transplantation associated diseases, including Graft rejection and Graft-Versus-Host-Disease (GVHD) are T lymphocyte-dependent; inhibition of T lymphocyte function is ameliorative.

Other diseases in which intervention of the immune and/or inflammatory response have benefit are infectious disease including but not limited to viral infection (including but not limited to AIDS, hepatitis A, B, C, D, E and herpes) bacterial infection, fungal infections, and protozoal and parasitic infections (molecules (or derivatives/agonists) which stimulate the MLR can be utilized therapeutically to enhance the immune response to infectious agents), diseases of immunodeficiency (molecules/derivatives/agonists) which stimulate the MLR can be utilized therapeutically to enhance the immune response for conditions of inherited, acquired, infectious induced (as in HIV infection), or iatrogenic (i.e. as from chemotherapy) immunodeficiency, and neoplasia.

Additionally, inhibition of molecules with proinflammatory properties may have therapeutic benefit in reperfusion injury; stroke; myocardial infarction; atherosclerosis; acute lung injury; hemorrhagic shock; burn; sepsis/septic shock; acute tubular necrosis; endometriosis; degenerative joint disease and pancreatis.

The compounds of the present invention, e.g. polypeptides or antibodies, are administered to a mammal, preferably a human, in accord with known methods, such as intravenous administration as a bolus or by continuous infusion over a period of time, by intramuscular, intraperitoneal, intracerebrospinal, subcutaneous, intra-articular, intrasynovial, intrathecal, oral, topical, or inhalation (intranasal, intrapulmonary) routes.

It may be desirable to also administer antibodies against other immune disease associated or tumor associated antigens, such as antibodies which bind to CD20, CD11a, CD 40, CD18, ErbB2, EGFR, ErbB3, ErbB4, or vascular endothelial growth factor (VEGF). Alternatively, or in addition, two or more antibodies binding the same or two or more different antigens disclosed herein may be coadministered to the patient. Sometimes, it may be beneficial to also administer one or more cytokines to the patient. In one embodiment, the polypeptides of the invention are coadministered with a growth inhibitory agent. For example, the growth inhibitory agent may be administered first, followed by a polypeptide of the invention. However, simultaneous administration or administration first is also contemplated. Suitable dosages for the growth inhibitory agent are those presently used and may be lowered due to the combined action (synergy) of the growth inhibitory agent and the polypeptide of the invention.

For the treatment or reduction in the severity of immune related disease, the appropriate dosage of an a compound of the invention will depend on the type of disease to be treated, as defined above, the severity and course of the disease, whether the agent is administered for preventive or therapeutic purposes, previous therapy, the patient's clinical history and response to the compound, and the discretion of the attending physician. The compound is suitably administered to the patient at one time or over a series of treatments.

L. Articles of Manufacture

In another embodiment of the invention, an article of manufacture containing materials useful for the diagnosis or treatment of the disorders described above is provided. The article of manufacture comprises a container and an instruction. Suitable containers include, for example, bottles, vials, syringes, and test tubes. The containers may be formed from a variety of materials such as glass or plastic. The container holds a composition which is effective for diagnosing or treating the condition and may have a sterile access port (for example the container may be an intravenous solution bag or a vial having a stopper pierceable by a hypodermic injection needle). The active agent in the composition is typically an IL-17 or LIF antagonist. The composition can further comprise any or multiple ingredients disclosed herein. The instruction on, or associated with, the container indicates that the composition is used for diagnosing or treating the condition of choice. For example, the instruction could indicate that the composition is effective of the treatment of osteoarthritis, rheumatoid arthritis or any other cartilagenous disorder. The article of manufacture may further comprise a second container comprising a pharmaceutically-acceptable buffer, such as phosphate-buffered saline, Ringer's solution and dextrose solution. It may further include other materials desirable from a commercial and user standpoint, including other buffers, diluents, filters, needles, syringes, and package inserts with instructions for use.

The following examples are offered for illustrative purposes only, and are not intended to limit the scope of the present invention in any way.

All patent and literature references cited in the present specification are hereby incorporated by reference in their entirety.

### EXAMPLES

Commercially available reagents referred to in the examples were used according to manufacturer's instructions unless otherwise indicated. The source of those cells identified in the following examples, and throughout the specification, by ATCC accession numbers is the American Type Culture Collection, Rockville, Md.

### Example 1

### Effect of Interleukin-17 on Cartilage Matrix Turnover

The experiments of this example examine the effect of IL-17 on cartilage matrix turnover. This effect is determined by measuring matrix (i.e. proteoglycan) synthesis and breakdown, as well as nitric oxide production, in articular cartilage. These parameters are evaluated in the presence and absence of interleukin 1α, IL-1α. Articular cartilage explants have several advantages over primary cells in culture. First, and perhaps most importantly, cells in explants remain embedded in tissue architecture produced in vivo. Secondly, these explants are phenotypically stable for several weeks ex vivo, during which time they are able to maintain tissue homeostasis. Finally, unlike primary cells, explants can be used to measure matrix breakdown. To set up cartilage explants, articular cartilage must be dissected and minced which results in disruption of the collagen network and release of proteoglycans into the culture media. This system thus mimics degenerative conditions such as arthritis in which the matrix is progressively depleted. Using this system, we have found that IL-17 can: (1) inhibit proteoglycan (PG) synthesis; (2) stimulate PG release; (3) enhance IL-1α-induced PG breakdown; (4) enhance the IL-1α-induced reduction in PG synthesis; (5) enhance both basal and IL-1α-induced nitric oxide production; and (6) induce the production of aggrecanase.

Il-1α has catabolic effects on cartilage including up-regulation of enzymes that induce matrix breakdown (matrix metalloproteinases and aggrecanases) as well as inhibition of synthesis of new matrix molecules (proteoglycans and collagens). Thus, the ability of the test compound to not only have negative effects on cartilage, but to also enhance the deleterious effects of IL-1α is strong evidence of the catabolic effect exhibited by IL-17. In addition, such an activity suggests that the test compound could enhance the degradation that occurs in arthritic conditions, since high levels of IL-1 are found in arthritic joints, and IL-1 function has been shown to be an important part of the progression of osteoarthritis. Arend W. P. et al., Ann. Rev. Immunol 16: 27-55 (1998).

### Example 1A

### Effect of IL-17 Upon Cartilage Matrix Metabolism

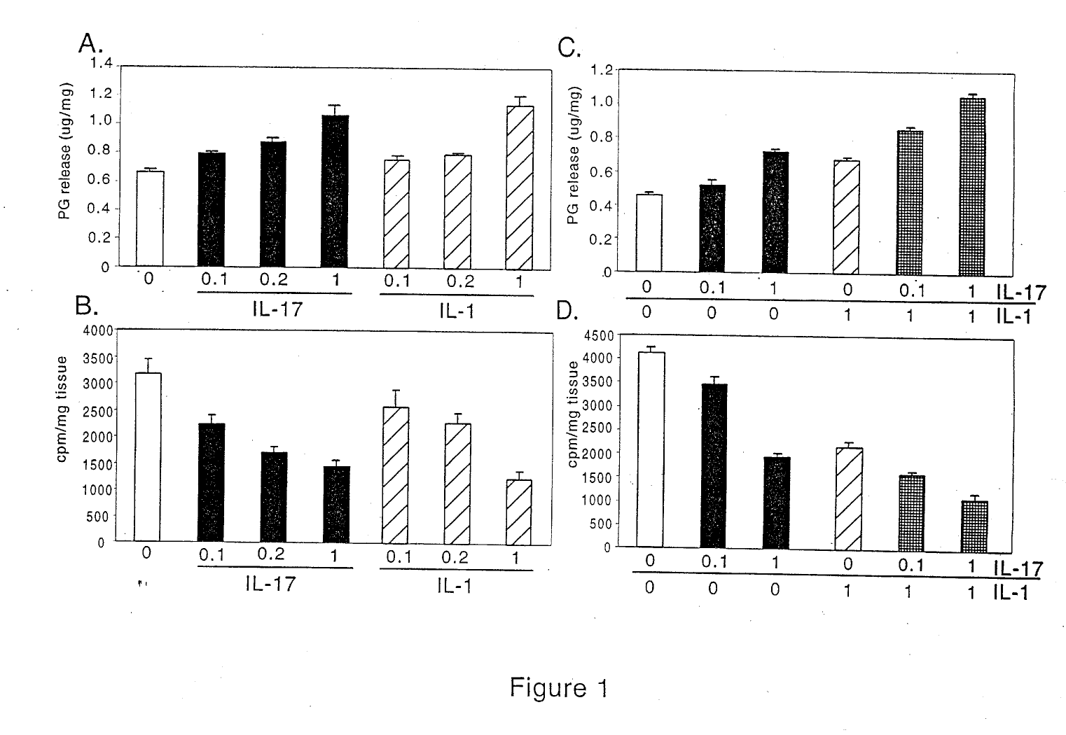

To determine whether IL-17 affects cartilage matrix metabolism, porcine articular cartilage explants were treated with a range of IL-17 concentrations, and proteoglycan synthesis and breakdown were measured. At concentrations as low as 0.1 ng/ml, IL-17 induced significant cartilage breakdown (FIG. 1A) and inhibited new matrix synthesis (FIG. 1B), with comparable potency to IL-1α. When IL-1α (1 ng/ml) and IL-17 (0.1 or 1 ng/ml) were combined, an enhancing, apparently additive, effect was observed on both matrix breakdown (FIG. 1C) and synthesis (FIG. 1D). Unlike what was found in a prior study (Chabaud et al., Arthritis Rheum. 42(5): 963-970 (1999), no synergism between IL-1α and IL-17 was observed.

To test for species-related effects, the ability of IL-17 to alter matrix metabolism in bovine articular cartilage explants was measured. While both IL-17 and IL-1α increased proteoglycan breakdown and inhibited matrix breakdown in a dose-dependent manner, bovine articular cartilage was less responsive than porcine tissue to IL-17 as evidenced by the difference in their response to low concentrations (≦1 ng/ml) of IL-17 (data not shown). Also tested was human cartilage harvested from patients with late-state OA, in which inhibition of matrix synthesis was more pronounced in response to IL-1α than to IL-17 (data not shown).

Thus, for all species tested, IL-17 was found to be a potent stimulator of articular cartilage catabolism.

### Example 1B

### IL-17 Induction of Catabolic Proteins

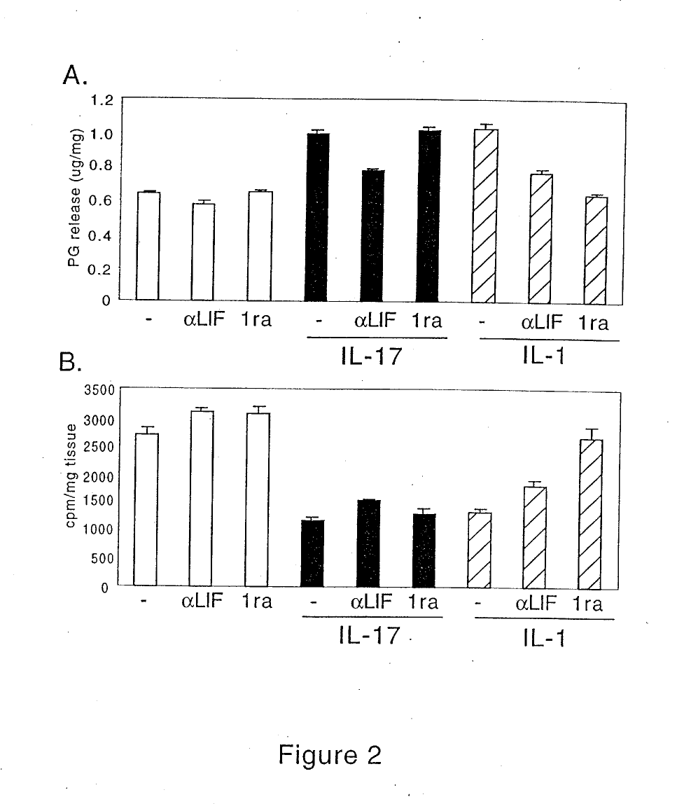

To determine the role of IL-1 in IL-17-induced matrix turnover, explants were treated with IL-17 plus IL-1α antagonist (IL-1ra). Although IL-1ra inhibited the effects of IL-1α on articular cartilage explants, IL-1ra neither blocked IL-17-induced matrix breakdown (FIG. 2A) nor prevented inhibition of matrix synthesis by IL-17 (FIG. 2B). Thus, the effects of IL-17 on matrix turnover were not dependent on IL-1 production by chondrocytes.

To determine the role of LIF in IL-17 activity, articular cartilage explants were treated with antibodies to LIF (anti-LIF) alone, or in combination with IL-17 or IL-1α inhibition of LIF significantly decreased IL-17 and IL-1α induced matrix breakdown (FIG. 2A), and partially overcame the inhibitory effects of IL-17 and IL-1α on matrix synthesis (FIG. 2B). The effect of anti-LIF on basal matrix turnover suggests that porcine explants synthesize active LIF under serum-free conditions (FIG. 2). Furthermore, IL-17- and IL-α-induced changes in cartilage matrix turnover appear to be mediated, at least in part, by LIF.

### Example 1C

### IL-17 Induction of Nitric Oxide (NO)

IL-17 also induces the production of nitric oxide (NO) which is believed to play a role in the pathology of cartilagenous disorders, including arthritis. Attur et al., Arthritis & Rheum. 40: 1050-1053 (1997).

High levels of nitrites are found in the synovial fluid of patients with osteo- or rheumatoid arthritis. Farrell et al., Ann. Rheum. Dis. 51: 1219-1222 (1992); Renoux et al., Osteoarthritis Cartilage 4: 175-179 (1996). Moreover, tissue explants from such patients spontaneously release high levels of nitrite in the absence of stimulation with cytokines such as IL-1. Amin et al., Curr. Opin. Rheum. 10: 263-268 (1998). Support for a causative role for nitric oxide in joint degeneration comes from studies showing the reduced arthritic progression in animals treated with agents which inhibit nitric oxide production by inhibiting nitric oxide synthase (NOS). Pelletier et al., Arthritis Rheum. 41: 1275-86 (1998); Pelletier et al., Osteoarthritis Cartilage, 7: 416-8 (1999. However, the determination of whether NO may play a positive or negative role in the progression of joint degeneration may depend upon the particular animal tested, in that in another animal model of arthritis, NOS inhibitors increased arthritic lesions. Sakiniene et al., Clin. Exp. Immunol. 110: 370-7 (1997).

Excessive nitric oxide within a damaged or diseased joint can affect not only the cells producing it, i.e., synovial cells and chondrocytes, but also leukocytes and monocyte-macrophages. In this way, NO can induce additional cytokine release, inflammation, and angiogenic activity. Amin and Abramson, Curr. Opin. Rheum. 10: 263-268 (1998). Blocking nitric oxide snythase (NOS) activity can attenuate the effects of IL-1β on matrix metalloproteinase production, aggrecan synthesis, and lactate production by chondrocytes. However, the role of NO in mediating the effect of other cytokines, such as IL-17, on cartilage matrix breakdown and synthesis has not yet been determined.

The assay for nitric oxide described herein is based on the principle that 2,3-diaminonapthalene (DAN) reacts with nitrite under acidic conditions to form 1-(H)-naphthotriazole, a fluorescent product. As NO is quickly metabolized into nitrite (NO₂⁻¹) and nitrate (NO₃⁻¹), detection of nitrite is one means of detecting (albeit undercounting) the actual NO produced in cartilagenous tissue.

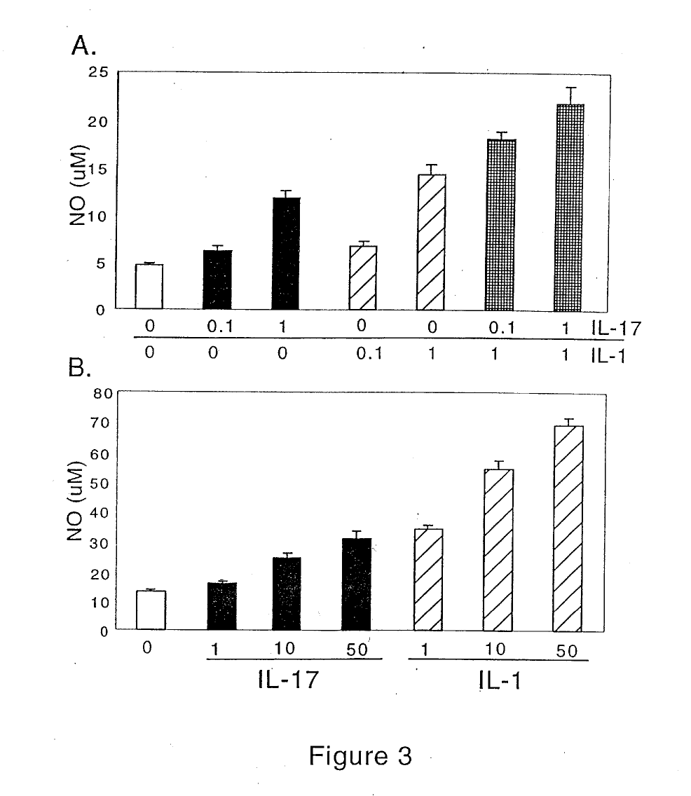

To elucidate the role of NO in IL-17 induced changes in cartilage matrix metabolism, nitric oxide production by articular cartilage explants was measured. In this system, IL-17 induced significant nitric oxide release in a dose-dependent manner (FIG. 3). IL-17 also augmented IL-1α induced nitric oxide production (FIG. 3A) in accordance with its ability to enhance the effects of IL-1α on matrix breakdown (FIG. 1C) and synthesis (FIG. 1D).

Consistent with the finding that bovine tissue was more sensitive to IL-1 than to IL-17 in terms of matrix turnover (data not shown), production of NO in response to IL-1 was greater than that produced after IL-17 treatment (FIG. 3B). Similarly, IL-17 at 1 ng/ml induced significant nitric oxide production in porcine (FIG. 3A), but not bovine (FIG. 3B) cartilage. Thus, in bovine tissue, production of nitric oxide requires treatment with relatively high levels of IL-17.

### Example 1D

### Role of Nitric Oxide (NO) in IL-17-Induced Effects

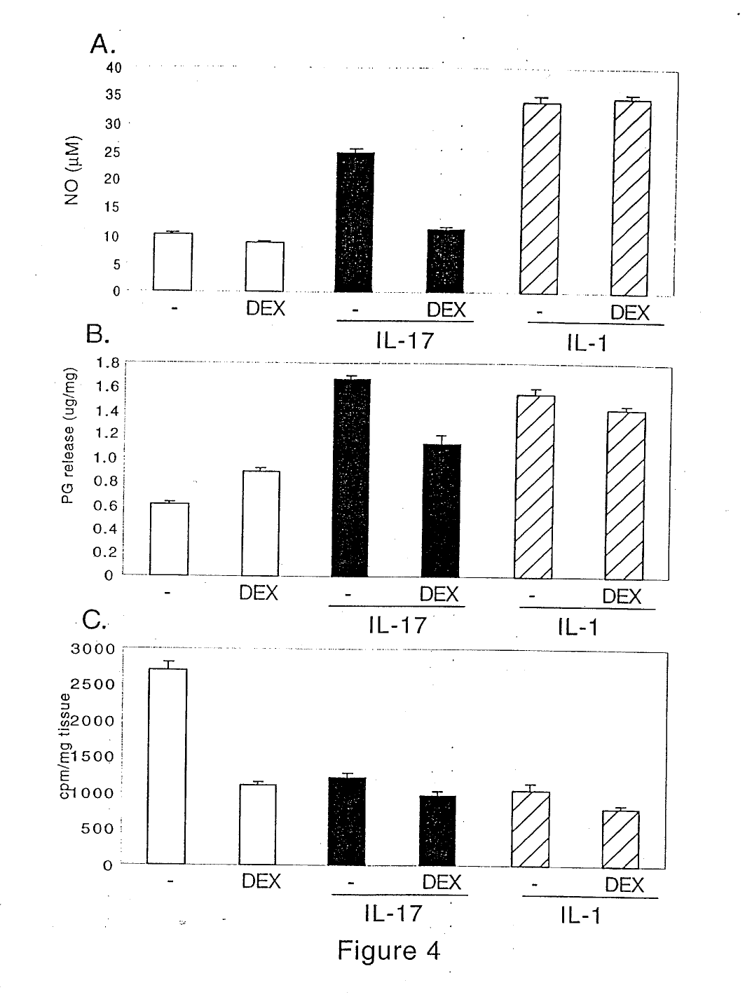

Dexamethasone (dex), a known inhibitor of IL-17-induced production of NO in human chondrocytes, was also examined in porcine articular cartilage explants. Porcine articular cartilage explants were treated with dex alone or in combination with IL-1α or IL-17. Within 24 hours of treatment, dex inhibited IL-17-, but not IL-α-, induced nitric oxide production (FIG. 4A). Similarly, dex inhibited IL-17-, but not IL-1α-, induced proteoglycan release (FIG. 4B). While dexamethasone by itself dramatically inhibited matrix synthesis, dex had very little effect on IL-17 or IL-1α induced inhibition of synthesis (FIG. 4C). Thus, IL-17-, but not IL-α-, induced production of NO and matrix breakdown is sensitive to the inhibitory effects of the anti-inflammatory dexamethasone.

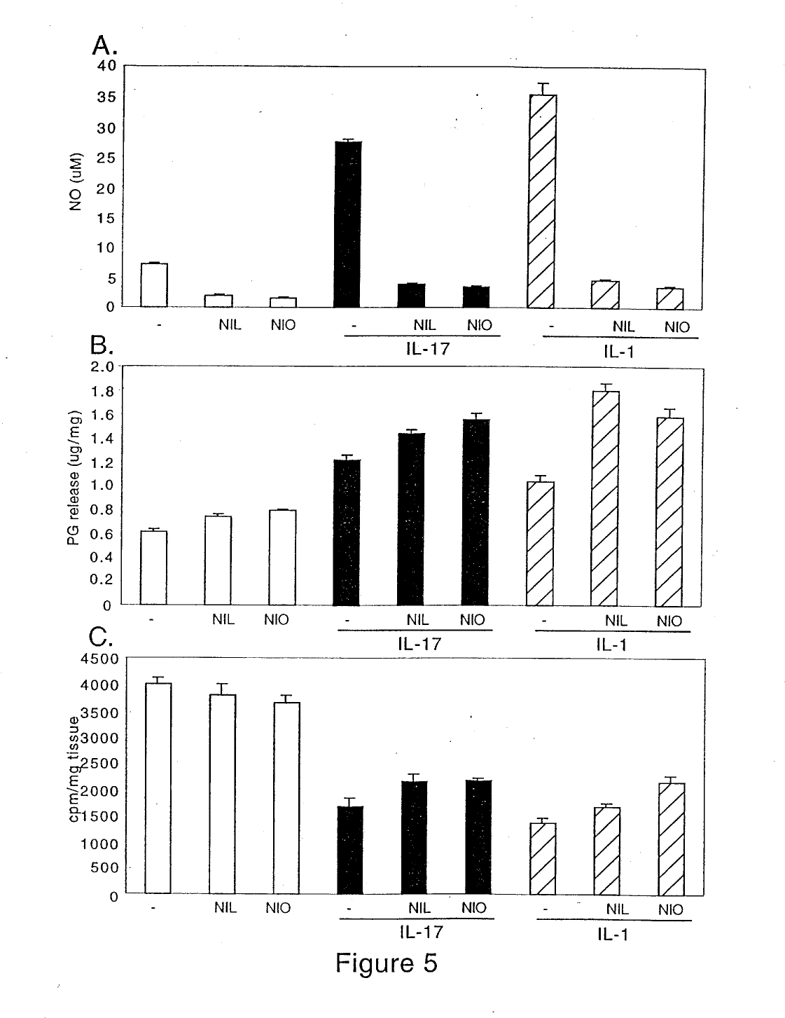

To clarify the role of NO in matrix metabolism, inhibitors of nitric oxide synthase (NOS), and thus nitric oxide production, were used to treat porcine explants, alone or in combination with IL-17 or IL-1α. These two inhibitors, L-NIL and L-NIO, completely suppressed both IL-17 and IL-1α induction of nitric oxide (FIG. 5A). Treatment with L-NIL or L-NIO led to a dramatic enhancement of IL-1α and IL-17 induced matrix breakdown (FIG. 5B) and a slight recovery in matrix synthesis (FIG. 5C) in cytokine treated tissues.

Thus, endogenous nitric oxide production appears to decrease cytokine-induced matrix breakdown, but may partially mediate cytokine inhibition of matrix synthesis.

### Example 1E

### IL-17 Induction of Aggrecanase

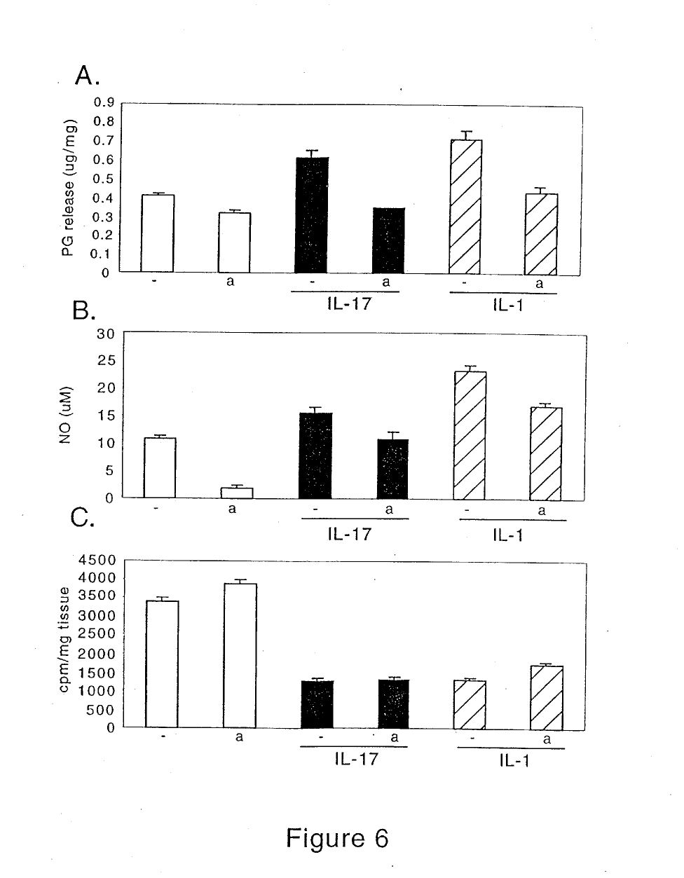

In order to determine which enzymes are involved in cytokine-induced matrix breakdown, a low concentration of actinonin was used. Actinonin can inhibit a recently isolated, site-specific, aggrecan-cleaving pretease termed “aggrecanase”, but not matrix metalloproteinase (MMP) activity. In articular cartilage explants, actinonin decreased basal and IL-1α or IL-17 induced matrix catabolism (FIG. 6A). Unexpectedly, actinonin decreased basal, as well as IL-1α and IL-17-, induced NO production (FIG. 6B).

The effects of actinonin were not due to cytotoxicity, as actinonin had no untoward effects on isolated primary chondrocytes in vitro nor on proteoglycan synthesis in articular cartilage explants (FIG. 6C). These results suggest that both IL-1α and IL-17 induced matrix breakdown is mediated by aggrecanase, and not by MMPs.

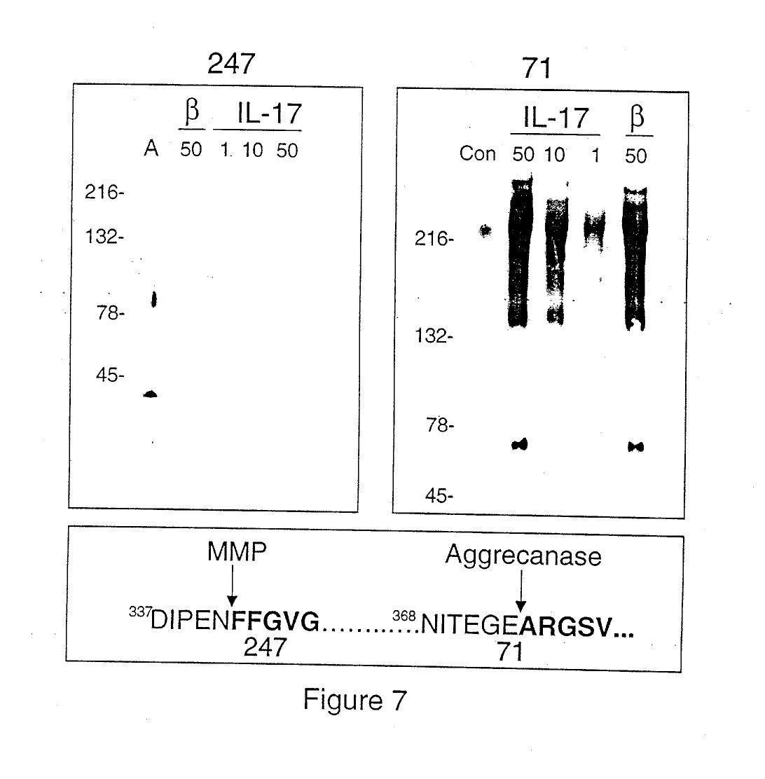

Two predominant catabolic sites are found within the interglobular domain (IGD) of aggrecan, the major proteoglycan in articular cartilage. One of these (between Asn³⁴¹ and Phe³⁴²) is believed to be due to MMP activity and the other (between Glu³⁷³ and Ala³⁷⁴) is likely due to “aggrecanase” activity. Analysis of the major proteoglycan degradation products released from cells can thus be used to determine which enzyme(s) have been activated. Western blot analysis of aggrecan fragments released into the media from tissues treated with IL-1 or IL-17 or control media was performed using antibodies which recognized either the new NH₂ terminal ARG-generated by aggrecanase (#71), or the new NH₂ terminal FFG-generated by MMP activity (#247) (FIG. 7). While MMP neo-epitopes were found in p-aminophenylmercuric acetate (APMA) treated explants as expected, no such fragments were found in explants treated with IL-1 or IL-17 (FIG. 7 left panel). In contrast, aggrecanase generated fragments were found in IL-1 and IL-17 treated samples, and the pattern of these fragments was similar (FIG. 7 right panel). Namely, bands of reactivity were detected at ˜230 kDa, ˜200 kDa, ˜150 kDa, ˜110 kDa and ˜64 kDa. The high molecular mass band at 230 kDa most likely represents the C-terminal aggrecan fragment formed by initial cleavage at the Glu³⁷³-Ala³⁷⁴ bond within the interglobular domain. Additional cleavage in the C terminus at other sites, likely accounts for the smaller products. Thus, both IL-1 and IL-17 induced aggrecanase, but not MMP, activity in articular cartilage explants.

### Example 1F

### Effect of IL-17 on MMP Expression in Cartilage Explants

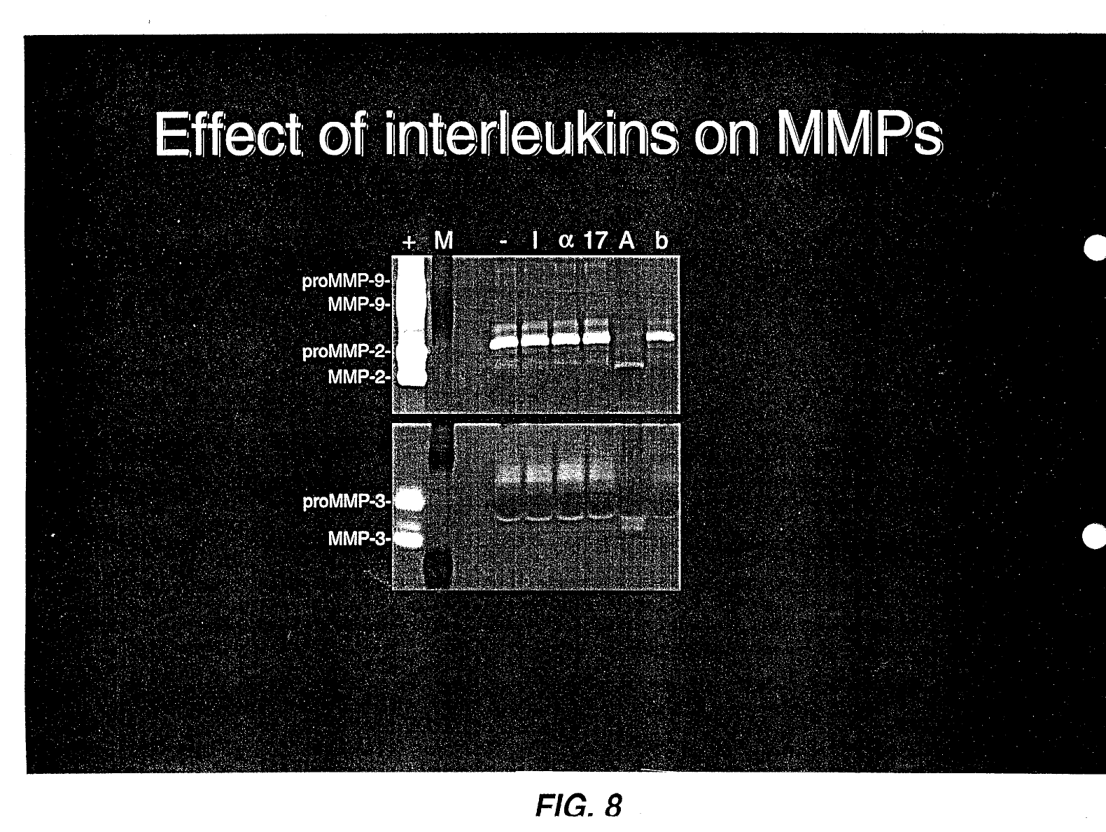

In order to further clarify which proteases are regulated by IL-1α and IL-17, the amount of MMPs in conditioned media from articular cartilage explants was determined. Media was analyzed by gel zymography as described in Materials and Methods. Articular cartilage explants expressed high levels of MMP-2, and much lower levels of MMP-9 (FIG. 8, top panels). Very little of the active form of these enzymes was found; however, APMA could activate pro-MMP-2 to its active MMP-2 form. Similarly, stromelysin (most likely MMP-3) was expressed predominately as a pro-MMP, but could be activated by APMA (FIG. 8, bottom panel). Consistent with the results from our Western blot analyses, no upregulation of MMP expression or activity was detected in IL-1α or IL-17 treated explants (FIG. 8).

### Example 2

### Effect of IL-17 on MMP Expression in Chondrocytes in Culture

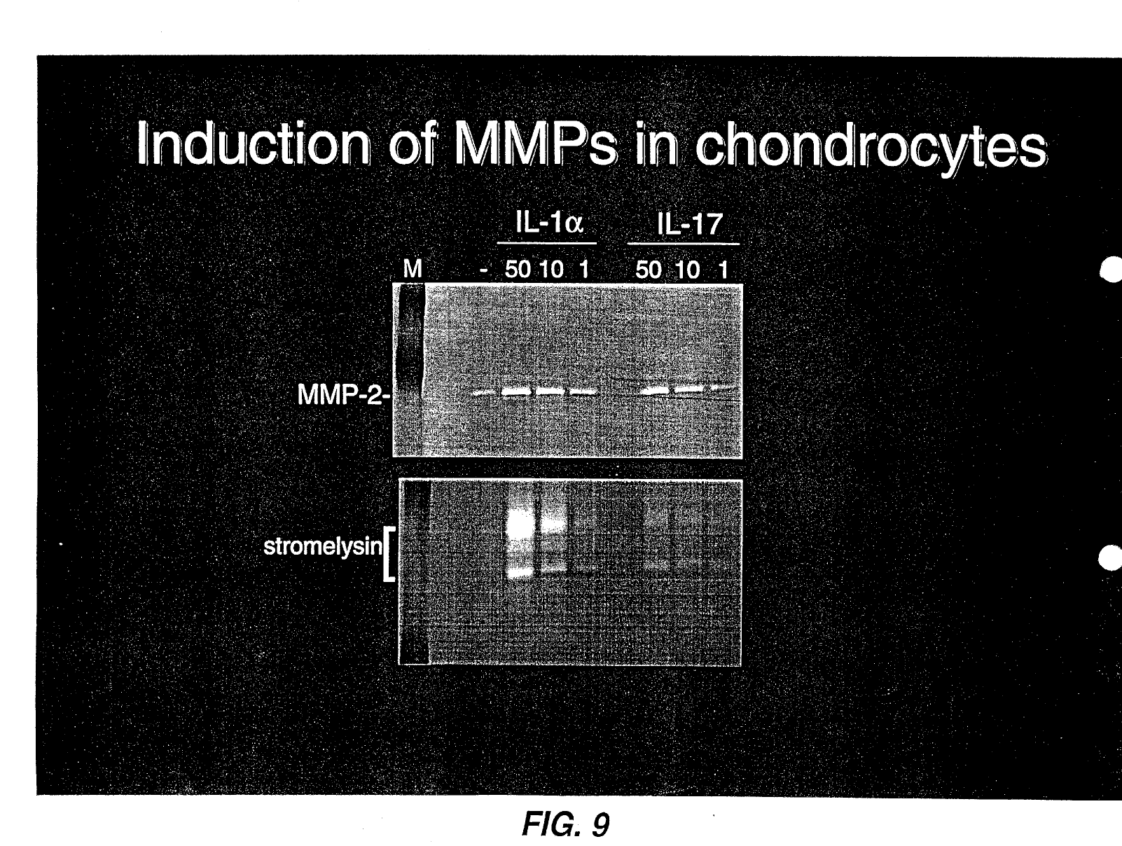

In order to culture chondrocytes, articular cartilage is digested with enzymes which remove the extracellular matrix. Thus, the cellular environment in this culture system may be similar to that found in later stages of cartilage disorders where the matrix has been depleted. Since essentially all of the MMPs synthesized by chondrocytes cultured in monolayer is secreted into the media, the amount of MMPs in the media of such cells is indicative of MMP production. MMPs were detected as described above for media from explants. As in cartilage explants, the primary MMPs expressed by chondrocytes were MMP-2 and stromelysin, and these were present as pro-enzymes (FIG. 9, left panel). However, unlike cells in cartilage explants, cells cultured as monolayers responded to either IL-1α or IL-17 by inducing expression of MMP-2 and stromelysin (FIG. 9, left panel).

### Example 3

### In Vivo Effects of IL-17

The patellar assay determines the in vitro and in vivo effect of the tested compound on proteoglycan synthesis in the patellae of mice. The patella is a very useful model to study the effects of the test compound because it permits the evaluation on cartilage which has not been removed from the underlying bone. Moreover, since each animal has one patellae in each leg, experiments can be performed using the contralateral joint as a control. This assay involves injection of a protein into the intra-articular space of a (mouse) knee joint, and subsequent harvest (within a few days after injection) of the patella (knee cap) for measurement of matrix synthesis. The procedure performed herein, has been previously used to measure effects of cytokines in vitro and in vivo (Van den Berg et al., Rheum. Int. 1: 165-9 (1982); Verschure P. J. et al., Ann. Rheum. Dis. 53: 455-460 (1994); and Van de Loo et al., Arthrit. Rheum. 38: 164-172 (1995)).

In the explant system, articular cartilage is dissected away from surrounding tissues. In order to test the effects of IL-17 in another physiologically relevant system, we treated intact skeletal elements, i.e. whole patellae, with IL-1α or IL-17 in vitro. In this patellar assay, IL-17 decreased matrix synthesis, but to a lesser extent than did IL-1α (data not shown).

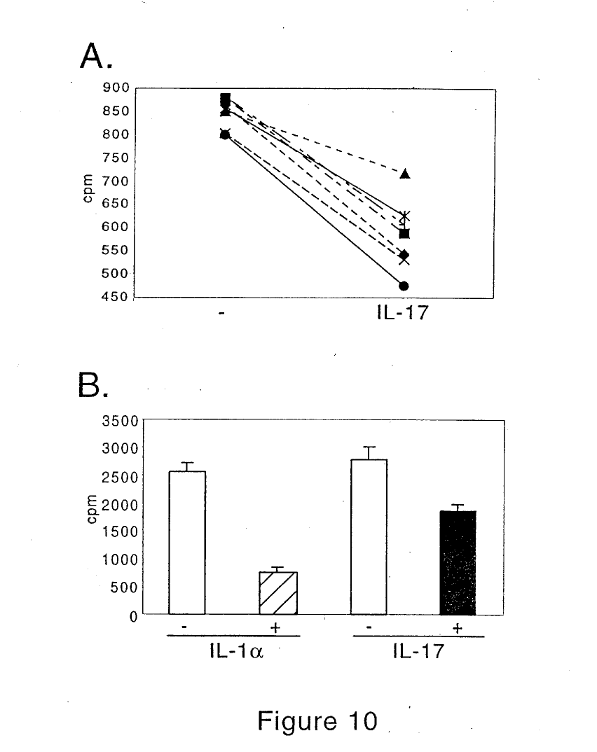

In order to test the in vivo effects, either IL-17 in buffer (PBS+0.1% bovine serum albumin) or buffer alone was injected into the intra-articular space of the knee joints of mice. Twenty-four hours after the last injection, patellae were harvested and proteoglycan synthesis was measured. Proteoglycan synthesis decreased significantly at high (80 ng) (FIG. 10A) but not low (12 ng) (data not shown), doses of IL-17. However, the extent of decrease with IL-17 at 80 ng (30%) was significantly less than that seen with IL-1α (70%), even when much lower doses of IL-1α (12 ng) were used (FIG. 10B).

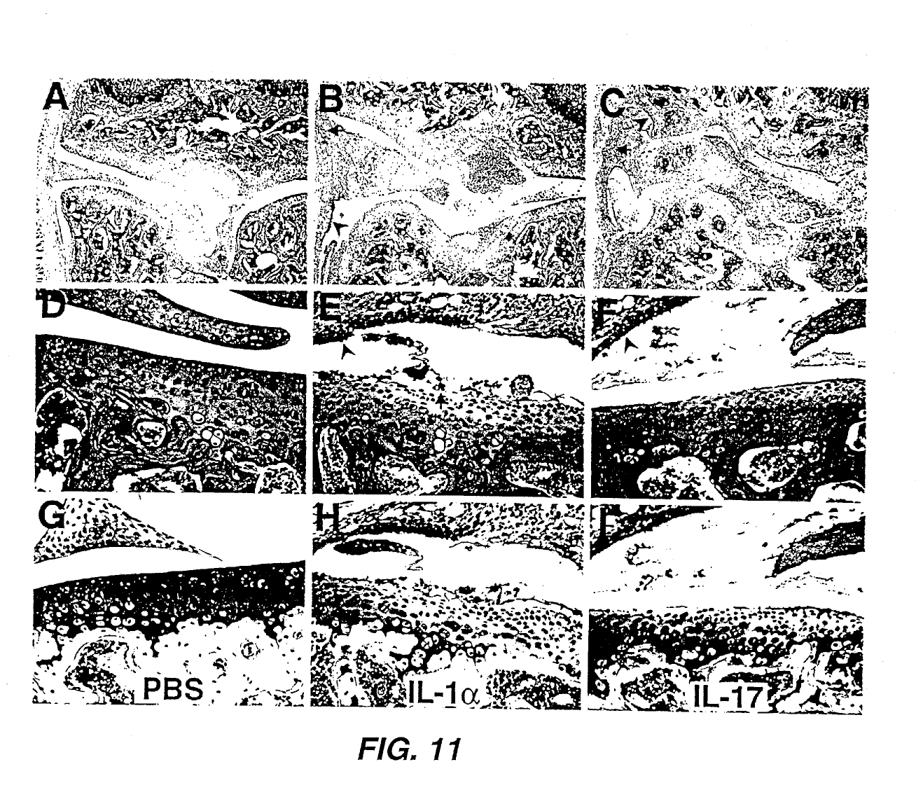

In order to better understand the in vivo effects of IL-17, joints injected with IL-17 (80 ng) were processed for histological examination. Joints injected with IL-1α (1 ng) were used as a positive control. Joints from PBS injected animals were normal or showed, at most, mild peri-articular reactive inflammation around the injection site (FIG. 11). Joints from all cytokine treated animals examined at day showed evidence of arthritis (FIGS. 11 B, C, E, F, H & I), characterized by moderate to severe inflammation of peri-articular tissues, reactive synovitis and intra-articular leukocyte infiltration. Leukocytes were often adherent to the surface of the articular cartilage and there was irregularity of the normally smooth articular surface. The inflammatory infiltrate contained both neutrophils and mononuclear cells; the morphologic features of the infiltrate were indistinguishable between the IL-1α and IL-17 treated animals. The intensity of safranin O staining (FIGS. 11G, H & I), which highlights the content of glycosaminoglycans in articular cartilage, was reduced in the most severely inflamed joints, when compared with controls.

In conclusion, both IL-1α and IL-17 can significantly decrease proteoglycan synthesis in vivo, which is consistent with the results seen in vitro. However, IL-17 appeared to be less potent than IL-1α in vivo in terms of decreased matrix synthesis. Histological analysis showed that IL-17 induces inflammation and leukocyte infiltration in a manner which resembles features of arthritic joints.

### Example 4

### Effect of Anti-IL-17 Antibodies in Animal Model of RA

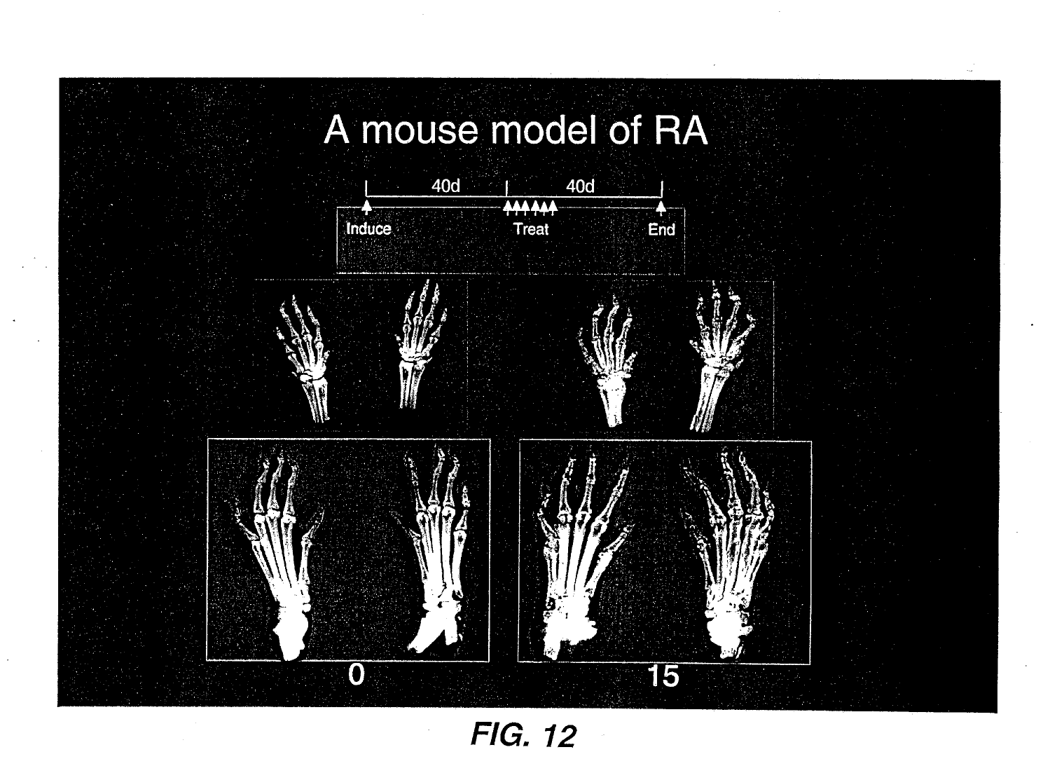

Injection of type II collagen into animals creates a specific immune reaction within synovial joints which resembles many of the features found in patients with RA. For example, in this collagen-induced arthritic model (CIA), animals have erosion of cartilage and bone at joint margins (FIG. 12), proliferative synovitis, symmetrical involvement of small and medium-sized peripheral joints in the appendicular, but not the axial, skeleton, Jamieson, T. W. et al., Invest. Radiol. 20: 324-9 (1985). Furthermore, IL-1 and TNF-α appear to be involved in CIA as in RA, Joosten et al., J. Immunol. 163: 5049-5055, (1999). In DBA1/LacJ strain, CIA was induced with bovine type II collagen, and approximately 40 days later, onset of the disease began. At this time, animals were treated with antibodies three times per week. Because the antibodies used were either rat (IL-17) or human (anti-TNFα), the mice could only be treated for 2 weeks with the antibodies. After this time, mice will raise antibodies to the exogenous antibodies (i.e. anti-IL-17 or anti-TNFα), so further treatment does not have the desired therapeutic effect. At approximately 40 days after the first injection of antibody (or 80 days after induction with collagen), animals were sacrificed and the joints examined. In the top panel of FIG. 12 is a diagram of the experimental plan. Below are the X-rays of the front (middle panel) or hind (lower panel) paws of the mice. During the course of the experiment, animals were given a clinical score based on redness, swelling, and number of joints affected. Shown are X-rays from animals with a score of 0 (no defects) or 15 (the worst). Loss of peri-articular bone, which is characteristic of RA, was clearly visualized in X-rays of mice with severe phenotypes (FIG. 12, “15”). Thus, in this model, inflammation and skeletal destruction can be examined.

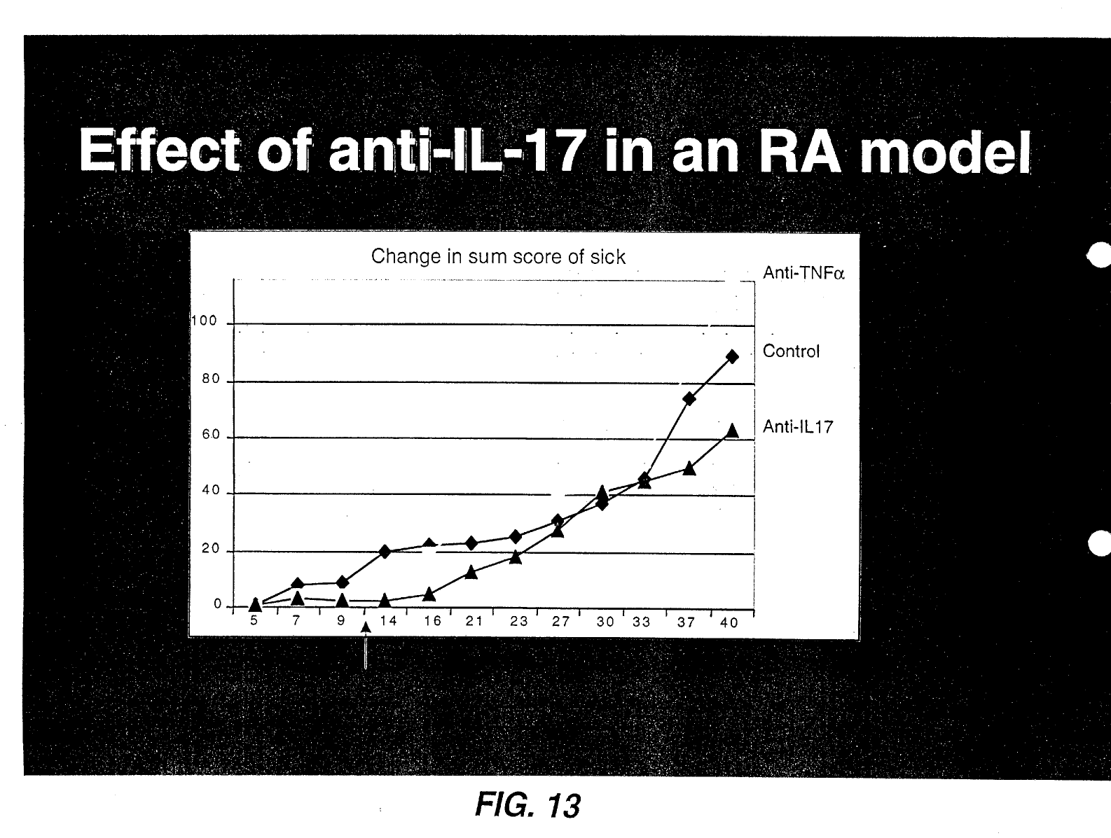

There were 12 animals in each treatment group, giving a sum score sick for each group on each day. The change in sum score sick represents progression of arthritis in that group of animals. As shown in FIG. 13, both anti-TNFα antibodies and anti-IL-17 antibodies decreased progression of arthritis during treatment. However, following cessation of treatment, the course of the disease in those animals previously injected with anti-TNFα antibodies appeared to be accelerated. In contrast, mice treated with anti-IL-17 antibodies continued to have a slower disease course than the control group (FIG. 13).

Thus, anti-IL-17 antibodies appear to be a good therapy for the treatment of arthritis as suggested by our result in an animal model of RA. Furthermore, anti-IL-17 antibodies may have advantages over anti-TNF-α antibodies. Given the different mechanism of action of IL-1 and TNFα in these animals models [Joosten et al., J. Immunol. 163: 5049-5055 (1999)], and the similarities between IL-1 and IL-17 activity (FIG. 1), it is likely that anti-IL-17 antibodies could not only augment the protective effects of anti-TNF-α antibodies in vivo, but likely provide superior therapeutic benefit in an in vivo treatment regimen.

Discussion (Examples 1A-F, 2, 3 and 4):

IL-17 as a Factor Involved in Joint Destruction

Cytokines are involved in the inflammation and cartilage destruction characteristic of arthritic disorders. IL-1 and TNF-α, which are present at high levels in diseased joints, induce cartilage matrix breakdown, expression of other pro-inflammatory molecules, and inhibit synthesis of new cartilage matrix proteins. Neutralizing TNF-α or IL-1 activity in vivo suppresses inflammation or protects skeletal tissues in arthritic animal models, Joosten et al., J. Immunol. 163: 5049-5055 (1999), Joosten et al., Arthritis Rheum. 39(5): 797-805 (1996), and attenuates clinical disease activity in humans. Elliott et al., Arthritis Rheum. 36: 1681 (1993), Bresnihan et al., Arthritis Rheum. 41: 2196 (1998). The finding that neither treatment is able to completely cure arthritis suggests that other factor(s) are involved in joint degeneration.

In humans, as well as in animal models, rheumatoid arthritis (RA) is characterized by leukocyte infiltration, synovitis, and pannus formation. Arend and Dayer, Arthritis Rheum. 38: 151-60 (1995). Activated T cells are sufficient, Kong et al., Nature 402: 304-308 (1999), and perhaps necessary, Panayi et al., Arthritis Rheum. 35: 729-735 (1992) for induction of cartilage and bone loss in animal models of RA. Cytokines released by activated T cells can activate macrophages, fibroblasts and other T cells, thus enhancing the local immune response and triggering synovitis.

Interleukin 17 (IL-17), which is produced by activated T cells, is a likely contributor to the pathogenesis of arthritis. Chabaud et al., Arthritis Rheum. 42: 963-970 (1999); Kotake et al., J. Clin. Invest. 103: 1345-1351 (1999); Ziolkowska et al., J. Immunol. 164: 2832-2838 (2000). IL-17 stimulates synoviocytes, Chabaud et al., J. Immunol. 161: 409-414, 1998, as well as other cell types, Fossiez et al., J. Exp. Med. 183: 2593-2603 (1996), to produce IL-6, IL-8, granulocyte/macrophage-colony stimulating factor (G-CSF) and inflammation mediators, such as prostaglandins (PGE₂). Such activity may be the mechanism whereby IL-17 regulates the communication between T cell and hematopoietic cells. Fossiez et al., supra. Inhibition of IL-17 dramatically reduces (by 80%) stimulation of osteoclast formation by conditioned media from RA synovial tissues. Kotake et al., supra. Thus, IL-17 may be the main cytokine responsible for induction of juxta-articular bone loss in the early stages of rheumatoid arthritis, Chabaud et al., 1999, supra; Kotake et al., supra.

IL-17 induces the production of pro-inflammatory cytokines by stromal cells and synoviocytes, some of which can enhance the effects of IL-17. Fossiez et al., supra; Chabaud et al., 1998, supra. IL-17 stimulates production of, and can synergize with, IL-1, TNF-α, and IL-6. Attur et al., supra; Jovanovic et al., J. Immunol. 160: 3513-21 (1998); Chabaud et al., 1998, 1999, supra. In human peripheral blood macrophages, IL-17 stimulates the production of TNF-α, IL-1, IL-12 and the matrix metalloproteinase stromelysin. Jovanovic et al., supra. IL-17 similarly induces mRNA expression of IL-1β, IL-6, stromelysin, inducible nitric oxide synthase (iNOS), and cyclooxygenase-2 (COX-2), in chondrocytes, Shalom-Barak et al., J. Biol. Chem. 273: 27467-473 (1998). Production of IL-17 may be the mechanism whereby IL-15, which is induced by TNF-α and IL-1β, exerts its proinflammatory properties in vivo. Ziolkowska et al., supra. Finally, IL-17 appears to play a role in both induction and expansion of the proinflammatory cytokine cascade, and, as such, IL-17 may initiate as well as amplify joint destruction characteristic of RA.

Loss of cartilage tissue in arthritic patients results from an imbalance between matrix breakdown and synthesis. Release of proteoglycans from articular cartilage leads to impaired chondrocyte function and cartilage biomechanics, and may contribute to loss of other matrix molecules such as collagens. This is the first report to show that IL-17 can act directly on articular cartilage to stimulate degradation of proteoglycans through induction of activity of aggrecanase(s), but not matrix metalloproteinases. The severe inflammation of peri-articular tissues, reactive synovitis, intra-articular leukocyte infiltration, and inhibition of proteoglycan synthesis, found in mouse joints injected with IL-17 further support the hypothesis that IL-17 can induce an arthritic phenotype in vivo. Unlike other T cell-derived cytokines, which are difficult to detect in RA synovium, IL-17 appears to be produced at high levels in RA joints (Aarvak et al., J. Immun. 162: 1246-1251 (1999); Chaubaud et al., Arthritis Rheum. 42: 963-970 (1999); Ziolkowska et al., J. Immunol. 164: 2832-2838 (1999). Thus, production of IL-17 may be responsible, at least in part, for the compromised articular cartilage volume and integrity which is one of the major clinical problems of arthritic patients.

Soluble factors made by T cells, monocytes and synovial fibroblasts may act in concert as these cell types are found in close proximity in RA synovium. The fact that IL-17 induces expression of other cytokines, such as TNF-α and IL-1α Chabaud et al., J. Immunol. 161: 409-414, (1998); Jovanovic et al., J. Immunol. 160: 3513-21 (1998), which are found at high levels in diseased joints, Arend and Dayer, Arthritis Rheum. 38: 151-60 (1995), raises the intriguing possibility that IL-17 is involved in the initiation of the inflammatory cascade in arthritis. Overproduction of IL-17 by human mononuclear cells is triggered by IL-1β and IL-15 (Ziolkowska et al., supra), and IL-17 is likely responsible for production of IL-6 (Chaubaud et al., supra) and LIF, Chabaud et al. J. Immunol. 161: 409-414 (1998) and induction of bone resorption (Kotake et al., J. Clin. Invest. 103: 1345-1351 (2000) by RA synovial tissues. Thus, IL-17 may be one of the primary catabolic cytokines in arthritis. IL-17 may also perpetuate the cycle of cytokine synthesis as overproduction of IL-17 by human mononuclear cells is triggered by IL-1β and IL-15 (Ziolkowska et al., supra). As described herein, IL-17 disrupted cartilage matrix homeostasis and augmented the detrimental effects of IL-1α on articular cartilage matrix turnover and nitric oxide production. Thus, the presence of IL-17 in a diseased joint can amplify the inflammatory cascade and exacerbate skeletal tissue breakdown in human joints.

While inflammation may not be the initiating event in osteoarthritis (OA), the episodic inflammation which occurs in clinical OA is believed to accelerate cartilage loss and exacerbate pain. The inflammatory cells (i.e., monocytes, macrophages, and neutrophils) which invade the synovial lining after injury and during inflammation may include activated T cells. Finally, nitric oxide, which is induced by IL-17, may play a key role in OA pathophysiology. Thus, IL-17 may be involved in joint destruction in OA, as well as in RA.

The high levels of LIF which have been found in synovial fluid of arthritic patients (Dechanet et al., Eur. J. Immunol. 12:3222-8 (1994) may be due to IL-17 expression by the synovivum Chabaud et al., J. Immunol. 161: 409-414 (1998). As with IL-17, LIF stimulates production of TNF-α and IL-1β Villiger et al., J. Clin. Invest. 91: 1575-81 (1993), and conversely these cytokines induce expression of LIF. Lotz, M. et al., J. Clin. Invest. 90: 888-96 (1992); Campbell et al., Arthritis Rheum. 36: 790-4 (1993); Hamilton et al., J. Immunol. 150: 1496-502 (1993). As shown herein, endogenous LIF production appears to mediate, at least in part, the effects of inflammatory cytokines such as IL-1α or IL-17 on articular cartilage. Thus, inhibition of LIF, for example through the use of antibodies, may prove to be a useful therapy for arthritis, either alone or in combination with other treatments.

IL-17, like IL-1α, significantly increased proteoglycan release and nitric oxide (NO) production, and decreased proteoglycan synthesis. Dexamethasone (dex) modulated the effects of IL-17, but not IL-1α, thereby suggesting different signaling pathways for these two cytokines. Dexamethasone inhibits synthesis of IL-6 and IL-8 (Tyler et al., Articular Cartilage and Osteoarthritis, Kuettner et al., Eds, Raven Press, Ltd., New York, pp 251-264 (1992) and, at high levels, decreases the spontaneous production of prostaglandin, but not NO, by articular cartilage explants. Amin et al., Curr. Opin. Rheum. 10: 263-268 (1998). Dex also prevents IL-17 induced IL-1, IL-6 and COX-2 mRNA expression, NF-κb binding, and activation of MAP kinases in cultured primary human chondrocytes, Shalom-Barak et al., J. Biol. Chem. 273: 27467-473 (1998). NF-κB and MAP kinases have been implicated in IL-17 induced signaling. However, the discrepancies between results in normal (Shalom-Barak et al., supra) versus OA (Martel-Pelletier et al., 1999) chondrocytes make it difficult to determine their relative importance in this process. Nevertheless, our results with dexamethasone raise the interesting possibility that the role of NF-κb, IL-6, COX-2, or MAP kinases in IL-17 induced signaling is distinct from that in IL-1α induced signaling. Furthermore, dex may inhibit factor(s) involved in IL-17 stimulation of NO production and aggrecanase activity, but not those mediating inhibition of proteoglycan synthesis. Finally, the potent inhibition of matrix synthesis by dexamethasone, as well as its specific inhibition of cartilage catabolism by IL-17, but not IL-1, may have important clinical implications, since glucocorticoids such as dex, are widely used to treat inflammatory disorders.

Proteases of the matrix metalloproteinase and aggrecanase families are believed to be responsible at least in part for the cartilage matrix degradation which occurs during joint destruction, Smith R. L., Front. Biosci. 4: d704-712 (1999). Synovial fluid (SF) or articular cartilage from arthritic patients contain proteoglycan fragments containing both MMP- and aggrecanase generated termini (Sandy et al., J. Clin. Invest. 89: 1512-1516 (1992); Lohmander et al., Arthritis Rheum. 36: 1214-22 (1993); Lark et al., J. Clin. Invest. 100: 93-106 (1997). While IL-17 induces MMP-3 mRNA in isolated human chondrocytes (Shalom-Barak et al., 1998) and MMP-9 in macrophages (Jovanovic et al., Arthritis Rheum. 43: 1134-1144 (2000), no evidence for MMP activity in IL-17 or IL-1 treated explants was found. Rather, as shown by examination of aggrecan fragments in the media and by inhibition of breakdown with actinonin, IL-17 or IL-α-induced release of matrix fragments from cartilage explants appears to be due to aggrecanase activity. Similarly, increased matrix catabolism in human OA cartilage correlates with aggrecanase, not MMP, activity (Little et al., 1999). Thus, IL-17 appears to activate the key enzyme(s) involved in human cartilage breakdown. However, upregulation of pro-MMPs occurred when isolated chondrocytes were cultured with IL-1α of IL-17. Thus, when chondrocytes are depleted of their surrounding matrix, as in late stages of cartilage degeneration in vivo, IL-1α or IL-17 can upregulate MMP expression.

The production of nitric oxide (NO) can be induced in a number of cell types by catabolic cytokines (reviewed in Amin and Abramson, Curr. Opin. Rheum. 10: 263-268 (1998). However, the ability of IL-17 to induce NO production appears to be cell-type dependent as human cartilage, Attur et al., Arthritis Rheum. 40: 1050-1053 (1997) but not human monocytes (Jovanovic et al., J. Immunol. 160: 3513-21 (1998) respond to IL-17 by increasing release of NO. While NO has also been implicated in joint destruction in arthritis (reviewed in Amin and Abramson, supra.), its exact function in cytokine-induced matrix turnover is not yet clear.

Our data suggests the nitric oxide has both protective and detrimental effects on cartilage. The finding of increased proteoglycan release and decreased synthesis without detectable enhancement of nitric oxide production at low concentrations of IL-1α or IL-17, or after treatment with IL-1α or IL-17 and NO inhibitors, suggest that induction of matrix catabolism does not depend on production of appreciable amounts of nitric oxide. Inhibition of endogenous nitric oxide production partially overcame IL-17 or IL-1α induced inhibition of proteoglycan synthesis in porcine cartilage. In contrast, blockade of iNOS production completely prevented the inhibitory effects of IL-1 on rabbit cartilage (Taskiran et al., Biochem. Biophys. Res. Commun. 200: 142-8 (1994), but did not alter IL-1 suppression in bovine cartilage (Stefanovic-Racic et al., J. Immunol. 156: 1213-20 (1996). Our results in porcine cartilage emphasize the importance of the species when determining the role of NO in mediating the effect of cytokines on proteoglycan synthesis.

Our data support the hypothesis that nitric oxide protects articular cartilage from cytokine (IL-1α or IL-17)-induced catabolism, which is likely mediated by “aggrecanase” in porcine articular cartilage explants. Our findings thus suggest that nitric oxide inhibits aggrecanase activity, just as NO appears to suppress MMP production in rabbits chondrocytes (Stadler et al., J. Immunol. 147: 3915-20 (1991). The relative contribution of the protective vs. detrimental effects of NO on cartilage may depend upon the degree and timing of NO production, and/or the presence of additional cytokines which modify the effect of NO within the joint. Accordingly, inhibition of NO production in vivo can exacerbate (Sakiniene et al., Clin. Exp. Immunol. 110: 370-7 (1997) or prevent IL-1 mediated cartilage destruction in vivo (Pelletier, J-P et al., Arthritis Rheum. 41: 1275-86 (1998); van de Loo et al., Arthritis Rheum. 41: 634-46 (1998); Stichtenoth and Frolich, Br. J. Rheumatol. 37: 246-57 (1998); Pelletier, J-P et al., supra.

Rather suprisingly, in addition to its ability to inhibit aggrecanase activity in our articular cartilage explants, actinonin significantly decreased NO production. Actinonin, a naturally occurring antibacterial agent, is a hydroxamate-containing inhibitor of metallo-enzymes, especially matrix metalloproteases. Thus, it is tempting to speculate that NO and MMPs regulate each other in a feedback loop. Such a conclusion results from the realization that just as NO regulates activation of MMPs (Stadler et al., J. Immunol. 147: 3915-20 (1991), so too might metal-dependent proteases such as MMPs or aggrecanases, be involved in NO production. However, actinonin can also regulate MAP-kinase p42/ERK2 expression and phosphorylation (Lendeckel et al., Biochem Biophys Res Commun. 252(1):5-9 (1998), as well as Wnt-5A expression (Lendeckel et al., Adv. Exp. Med. Biol. 477: 35-41 (2000) [Lendeckel-2]. Since MAP kinases likely play a role in cytokine induced signaling (Shalom-Barak et al., J. Biol. Chem. 273: 27467-473 (1998); Martel-Pelletier et al., Arthritis Rheum. 42: 2399-2409 (1999), and Wnt5A affects chondrocyte maturation in the developing skeleton, Hartmann and Tabin, Development 127: 3141-3159 (2000), these molecules may mediate, at least in part, the effects of actinonin on NO production and proteoglycan synthesis.

Our results showing the opposite effect of iNOS inhibitors on matrix breakdown versus synthesis suggest that the role of nitric oxide in cytokine-induced matrix breakdown is different from that in cytokine-induced inhibition of matrix synthesis.

Finally, our results using anti-IL-17 antibodies in an animal model of RA suggests that anti-IL-17 antibodies could be a useful treatment of arthritis in humans. Although TNF-α inhibitors, which are currently used in clinical practice, offer some potential clinical benefit, anti-TNF-α antibodies do not inhibit tissue destruction in animal models, (Joosten et al., J. Immunol. 163: 5049-5055 (1999). Thus, TNF-α inhibitors may not prevent the long-term outcome of joint destruction in arthritic patients. In contrast, anti-IL-17 antibodies, like inhibitors of IL-1, Joosten et al. supra., may be able to offer protection against tissue destruction, and by themselves, or in combination with TNF-α inhibitors, suppress inflammation thereby offering therapeutic advantage in the treatment of cartilagenous disorders.

Summary:

The present study demonstrates that IL-17, a T cell derived cytokine, has direct effects on cartilage matrix metabolism and nitric oxide production. Cartilage matrix catabolism induced by IL-17 or IL-α appears to be mediated by member(s) of the aggrecanase family and not by those of the MMP family. While the effects of IL-17 on cartilage matrix turnover and NO production are similar to those of IL-1α, the downstream signaling pathways are not identical. Production of nitric oxide by articular cartilage in response to cytokines may serve to protect the tissue from matrix breakdown, while simultaneously inhibiting the synthesis of new matrix-building proteoglycan molecules.

The presence of IL-17 at high levels in diseased joints induces synoviocytes and macrophages to produce other cytokines, and also stimulates bone resorption. In animal models, inhibition of IL-1 prevents skeletal tissue destruction, while the inhibition of TNF-α activity alleviates the inflammatory component of arthritis. Unlike these two cytokines, blockade of IL-17 may prove to prevent degradation of cartilage and bone as well as joint inflammation in arthritis. Our results in animal models of RA support the hypothesis that anti-IL-17 antibodies will prove to be a useful treatment for patients with arthritis. Neutralizing antibodies or small molecule inhibitors of IL-17 activity could be used alone or in combination with existing therapies for patients with rheumatoid or osteo-arthritis.

Materials and Methods:

Reagents:

L-Ornithine, N⁵-(1-iminoethyl)-, dihydrochloride (L-NIO) (Cat. No.: 80320) and L-lysine, N⁶-(1-iminoethyl)-, dihydrochloride (L-NIL) (Cat. No.: 80310) were purchased from Cayman chemical (Ann Arbor, Mich. Peptide antibodies which recognized neoepitopes on cleaved aggrecan proteins were kindly provided by Dr. John S. Mort, Shriners Hospital for Children, Montreal, Quebec, Canada. Anti-IL-17 antibodies (MAB421), and recombinant cytokines (IL-4, IL-13, IL-17, IL-1α, IL-1β) were purchased from R&D Systems (Minneapolis, Minn.). Anti-LIF antibodies were produced at Genentech, Kim et al., J. Immunol. Methods 156:9-17 (1992). Dexamethasone was purchased from Sigma (Cat. No.: D-2915, St. Louis, Mo.). P-aminophenylmercuric acetate (APMA) was purchased from Aldrich (Cat. No.: 10556-2, Milwaukee, Wis.). Gels impregnated with casein or gelatin were purchased from Novex (Cat. No.: gelatin gels, EC61752; Casein gels, EC64052; San Diego, Calif.).

Articular Cartilage Explants

The metacarpophalangeal joint of 4-6 month old female pigs was aseptically opened, and articular cartilage was dissected free of the underlying bone. The cartilage was minced, washed and cultured in bulk for at least 24 hours in a humidified atmosphere of 95% air and 5% CO₂ in serum free low glucose 50:50 DMEM:F12 media with 0.1% BSA, 100 U/ml penicillin/streptomycin (Gibco), 2 mM L-glutamine, 1×GHT, 0.1 mM MEM Sodium Pyruvate (Gibco), 20 μg/ml Gentamicin (Gibco), 1.25 mg/L Amphotericin B (Sigma), 5 μg/mL Vitamin E and 10 μg/mL transferrin. Approximately 50 mg of articular cartilage was aliquoted into Micronics tubes and incubated for at least 24 hours in above media before being changed into media without Vitamin E and transferrin. Test proteins were then added. Media was harvested and changed at various time points (0, 24, 48, 72 h).

Chondrocyte Preparation:

The metacarpophalangeal joints of 4-6 month old female pigs were aseptically dissected, and articular cartilage was removed by free-hand slicing taking care so as to avoid the underlying bone. These cartilage fragments were then digested with 0.05% trypsin in serum-free Ham's F12 for 25 minutes at 37° C. The medium was drained and discarded, and cartilage was digested in 0.3% collagenase B in serum-free Ham's F12 media for 30 minutes at 37° C. The medium was drained and discarded, and the cartilage was digested overnight in 0.06% collagenase B in Ham's F12+10% fetal bovine serum. The cells were then filtered through a 70 micron nylon filter and seeded in Ham's F12 medium with serum.

Culturing of Chondrocytes:

Chondrocytes (prepared as described above) were grown in microtiter plates (Falcon microtest 96, flat bottom) at a density of 100,000 cells per well in media composed of Ham's F12 with antibiotics (10 μg/ml gentamicin, 250 ng/ml amphotericin B, 100 U/ml penicillin/streptomycin) in a final volume of 250 μl per well, for 6 days at 37° C. and 5% CO₂. Media was removed and used to measure proteoglycans at days 3 and 6.

Measurement of Proteoglycans:

DMMB is a dye that undergoes metachromasia (a change in color, in this case from blue to purple) upon binding to sulfated glycosaminoglycans (GAG), the side-chains of proteoglycans. The addition of sulfated proteoglycans to DMMB causes a decrease in the peak values at 590 and 660 nm with an increase in absorbance at 530 nm. Thus, the amount of proteoglycans in media was determined by adding DMMB dye in a 96 well plate format, and the change in color was quantitated using a spectrophotometer (Spectramax 250). The DMMB assay is a well-accepted method to measure the amount of proteoglycans in cartilage cultures. For this assay, a standard curve was prepared using chondroitin sulfate ranging from 0.0 to 5.0 μg. The procedure has been adapted from the colorimetric assay described in Farndale and Buttle, Biochem. Biophys. Acta 883: 173-177 (1986).

Measurement of Proteoglycan Synthesis in Articular Cartilage Explants

At 48 hr, ³⁵S-sulfate (to a final concentration of 10 μCi/ml) was added to the cartilage explants. After an overnight incubation at 37° C., media was saved for measurements of nitric oxide or proteoglycan content. Cartilage pieces were washed two times using explant media. 900 μL digestion buffer containing 10 mM EDTA, 0.1 M Sodium phosphate and 1 mg/ml proteinase K (Gibco BRL) was added to each tube and incubated overnight in a 50° C. water bath. 600 μL of the digest was mixed with 600 μL of 10% w/v cetylpyridinium chloride (Sigma). Samples were spun at 1000×g for 15 min. The supernatant was removed, and 500 μL formic acid (Sigma) was added to the samples to dissolve the precipitate. Solubilized pellets were transferred to scintillation vials containing 10 ml scintillation fluid (ICN), and samples were read in a scintillation counter.

Measurement of Nitric Oxide (NO)

10 μL of 0.05 mg/ml 2,3-diaminonapthalene (DAN) in 0.62M HCl was added to 100 μL media from cartilage explants. Samples were mixed and incubated at room temperature for 10-20 minutes. The reaction was terminated with 5 μL of 2.8 M NaOH. The fluorescent product, 2,3-diaminonaphthotriazole, was measured using a Cytoflor fluorescent plate reader with excitation at 360 nm and emission read at 450 nm.

Western Blot Analysis of Aggrecan Fragments

Cetylpyridinium chloride (CPC) was added to culture media from explants treated for 3 days to a final concentration of 1% (w/v). Precipitated proteoglycans and proteoglycan fragments were collected by centrifugation. The pellet washed with 1% (w/v) CPC then dissolved in isopropanol/water (3:2, v/v). Two volumes of ethanol saturated with potassium acetate were added at 4° C., and the proteoglycan samples (now as their potassium salts) were collected by centrifugation. The pellet was then washed twice with ethanol, then with ether, and air dried.

Proteoglycan samples were dissolved at 10 mg/ml in 0.1 M Tris/acetate, pH 7.0, containing 10 mM EDTA, 10 mM iodoacetamide, 5 mM phenylmethanesulphonyl fluoride, 0.36 mM pepstatin A, 0.24 unit/ml keratanase I (endo β-galactosidase, Sigma) and 0.12 unit/ml chondroitinase ABC and incubated at 37° C. overnight. Digestion was terminated by addition of SDS/PAGE sample buffer and incubation for 3 minutes in a boiling water bath. Samples were analyzed on 4-12% SDS/PAGE gradient gels followed by electroblotting to nitrocellulose membranes (Novex), which were probed with 1:1000 dilution of an antibody raised in rabbit against the ovalbumuin-conjugated peptide ARGSVIGGC or FFGVGAKKGC. Subsequently, membranes were incubated with sheep anti-rabbit Ig horseradish peroxidase conjugate (Amersham Life Science) and aggrecan catabolites were visualized by incubation with the SuperSignal West Pico Chemiluminescent Substrate (Pierce) for 5 minutes and then exposure of blots to film.

In Vivo Injections of IL-17:

Recombinant murine IL-1α or IL-17 (R&D Systems) in a volume of 3 μl in buffer [phosphate buffered saline (PBS) with 0.1% bovine serum albumin (BSA, Sigma)] was injected through the intrapatellar ligaments into the joint space of C57B16 mice. Buffer alone (PBS with 0.1% BSA) was used as a control. Mice were killed the day after the last injection of protein, and patellae were either harvested for measurements of proteoglycan synthesis, or included in the joint tissues fixed for histological analysis.

Histological Analysis:

Following sacrifice of animals, knees were fixed in 4% buffered formalin, followed by decalcification in Formical for 4-8 hours. Samples were then processed for paraffin embedding and for histological assessment. Three-micron thick step sections were cut in the coronal plane and stained with hematoxylin and eosin or safranin O.

Measurement of Proteoglycan Synthesis in Isolated Patellae:

Cartilage proteoglycan synthesis was measured by sulfate incorporation into patellae ex vivo. Briefly, patellae were dissected away from the patellar tendon and other soft tissues, labeled with ³⁵S sulfur (30 μCi/ml), washed and fixed in 4% buffered formalin overnight. Patellae were then decalcified in 5% formic acid for 4 hours, and cartilage was dissected away from underlying bone. The patellar cartilage was then transferred to scintillation vials containing 500 μl of the solubilizer Solvable (Packard Bioscience, Meriden, Conn.) and incubated at 60° C. for 1.5 hours. 10 ml of scintillation fluid (HIONIC-fluor, Packard Bioscience, Meriden, Conn.) was added, and samples were counted.

Zymography:

Conditioned media from cartilage explant culture or primary chondrocyte culture were mixed with NOVEX® Tris-Glycine SDS sample buffer (2×) let stand 10 minutes at room temperature (19-24° C.). Samples were applied to 10% Zymogram (Gelatin) gels or 12% Zymogram (Casein) gels and run for about 180 minutes at 125V. After running, gels were first incubated in 1× NOVEX Zymogram Renaturing Buffer for 30 minutes at room temperature with gentle agitation, then in 1× NOVEX® Zymogram Developing Buffer for 30 minutes at room temperature with gentle agitation. Fresh 1× developing buffer was then added and gels were incubated overnight at 37° C. for maximum sensitivity. GelCode Blue Stain Reagent from Pierce was used to stain the gels. Areas of protease activity will show up as clear bands.

Animal Models of Rheumatoid Arthritis:

DBA-1LacJ mice (7-8 weeks old) were immunized with 100 μg bovine type II collagen intra-dermally (in the base of the tail). At disease onset (at day 40), mice with no obvious inflammation or redness were selected and divided into separate groups of 12. To neutralize TNF-α, mice were injected i.p. three times/week for two weeks with 100 μg of anti-TNFα antibody (Enbrel). To eliminate IL-17 activity, 100 μg of anti-IL-17 antibodies (MAB421, R&D Systems) was injected three times/week for 2 weeks. Mice were sacrificed approximately 40 days after the first injection of antibody. Mice were carefully examined three times a week for the visual appearance of arthritis in peripheral joints, and scores for disease activity were given as previously described. van den Berg et al., Clin. Exp. Immunol. 95: 237-248 (1994). The clinical severity of arthritis (arthritis score) was graded on a scale of 0-2 for each paw, according to changes in redness and swelling, and the number of joints affected was counted.

Production of Anti-IL-17 and Anti-LIF Antibodies:

The monoclonal antibody MAB421 (R&D Systems) used for the initial in vivo studies was produced from a murine hybridoma elicited from a rat immunized with purified E. coli-derived, recombinant mouse interleukin 17 (rmIL-17). The IgG fraction of the tissue culture supernatant was purified by Protein G affinity chromotagraphy. This antibody has been selected for its ability to neutralize the biological activity of rmIL-17.

Anti-LIF or anti-IL-17 antibody was also produced using the procedure described as follows. Mice were immunized intraperitoneally with recombinant human IL-17 (made as an IgG fusion protein in baculovirus) or with human LIF (expressed in E. coli or in Chinese hamster ovary (CHO cells) at Genentech. Spleen cells obtained from these immunized mice were fused with mouse myeloma cells using 35% polyethyleneglycol. Hybridoma cell lines secreting antibody specific for human IL-17, which did not cross-react with CD4-IgG, were selected by ELISA, cloned at least twice by limiting dilution and further characterized. Ascites were produced in mice and monoclonal antibodies were purified using protein G conjugated Sepharose 4B.

### Example 5

### Anti-LIF Abs

The isolation and production of anti-LIF antibodies useable with the present method is described in U.S. Pat. No. 5,688,681 issued on Nov. 18, 1997 to Kim.

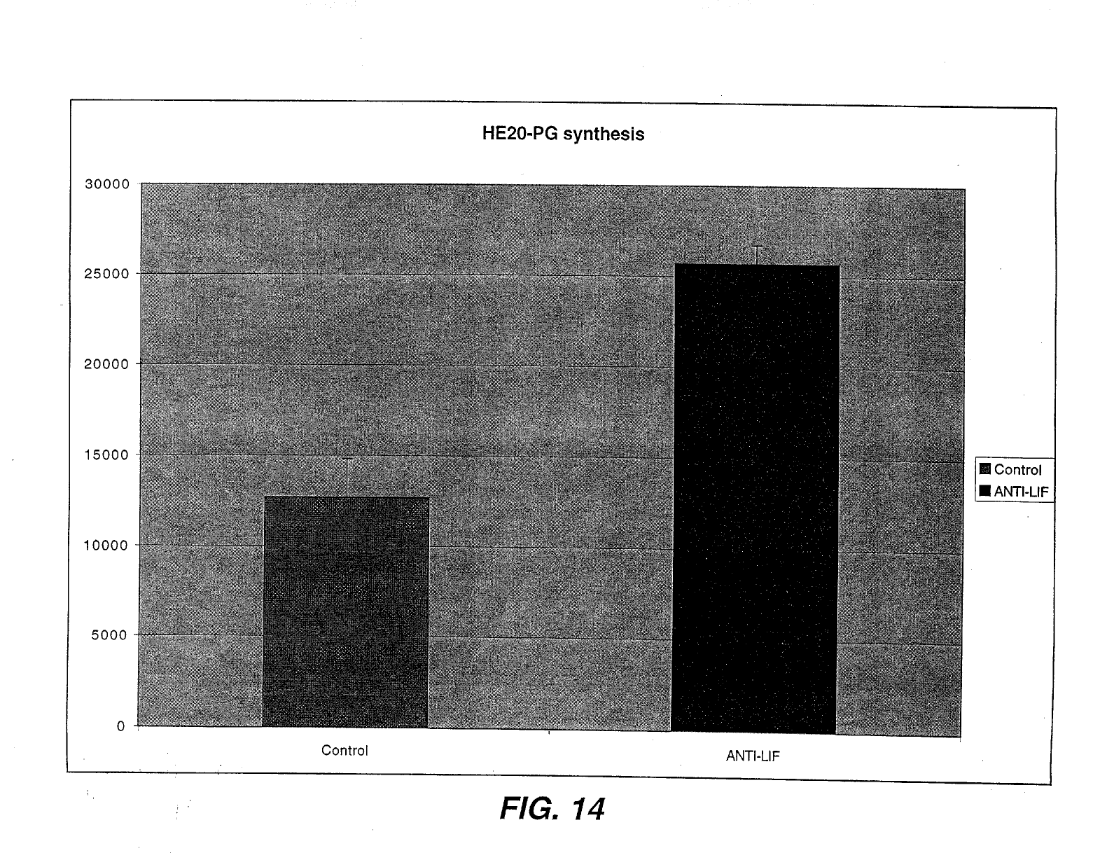

As shown in FIG. 14, treatment of human OA cartilage with anti-LIF antibodies resulted in a significant (almost 2×) increase in cartilage matrix synthesis. These results suggest that constitutive production of LIF by OA cartilage results in depressed matrix synthesis and that inhibition of LIF activity can overcome this negative autocrine loop. Thus, treatment of arthritic patients with anti-LIF antibodies is likely to result in upregulation of matrix synthesis in vivo and as such, may prove to be a useful method for the repair of cartilage.

### Example 6

### Articular Cartilage Explant Assay (Alternative)

Alternatively, the articular cartilage explant assay may be executed in a manner as described below.

Introduction:

As mentioned previously, IL-17 is likely to play a role in the initiation or maintenance of the proinflammatory response. IL-17 is a cytokine expressed in CD4⁺ Tₕ cells and induces the secretion of proinflammatory and hematopoietic cytokines (e.g., IL-1β, TNF-α, IL-6, IL-8, GM-CSF. Aarvak et al., J. Immunol. 162: 1246-1251 (1999); Fossiez et al., J. Exp. Med. 183: 2593-2603 (1996); Jovanovic et al., J. Immunol. 160: 3513-3521 (1998) in a number of cell types including synoviocytes and macrophages.

Expression of IL-17 has been found in the synovium of patients with rheumatoid arthritis, psoriatic arthritis, or osteoarthritis, but not in normal joint tissues. IL-17 can synergize with the monocyte-derived, proinflammatory cytokines IL-1β or TNF-α to induce IL-6 and GM-CSF. By acting directly on synoviocytes, IL-17 could enhance secretion of proinflammatory cytokines in vivo and thus exacerbate joint inflammation and destruction.

To further understand the possible role of IL-17, Applicants have tested the effect of IL-17 on cartilage matrix metabolism. In light of the catabolic effects of nitric oxide (NO) on cartilage, and the existence of high levels of NO in arthritic joints, NO production was also measured.

Methods:

Articular cartilage explants: The metacarpophalangeal joint of a 4-6 month old female pigs was aseptically dissected, and articular cartilage is removed by free-hand slicing in a careful manner so as to avoid the underlying bone. The cartilage was minced and cultured in bulk for at least 24 hours in a humidified atmosphere of 95% air 5% CO₂ in serum free (SF) media (DMEM/F12, 1:1) with 0.1% BSA and antibiotics. After washing three times, approximately 80 mg of articular cartilage was aliquoted into micronics tubes and incubated for at least 24 hours in the above SF media. Test proteins were then added at 1% either alone or in combination with IL-1α (10 ng/ml). Media was harvested and changed at various timepoints (0, 24, 48, 72 hours) and assayed for proteoglycan content using the 1,9-dimethyl-methylene blue (DMB) calorimetric assay described in Farndale and Buttle, Biochem. Biophys. Acta 883: 173-177 (1986). After labeling (overnight) with ³⁵S-sulfur, the tubes were weighed to determine the amount of tissue. Following an overnight digestion, the amount of proteoglycan remaining in the tissue as well as proteolgycan synthesis (³⁵S-incorporation) was determined.

Measurement of NO production: The assay is based on the principle that 2,3-diaminonapthalene (DAN) reacts with nitrite under acidic conditions to form 1-(H)-naphthotriazole, a fluorescent product. As NO is quickly metabolized into nitrite (NO₂⁻¹) and nitrate (NO₃⁻¹), dection of nitrite is one means or detecting (albeit undercounting) the actual NO produced. 10 μL of DAN (0.05 mg/mL in 0.62M HCl) is added to 100 μL of sample (cell culture supernatant), mixed, and incubated at room temperature for 10-20 minutes. Reaction is terminated with 5 μL of 2.8N NaOH. Formation of 2,3-diaminonaphthotriazole was measured using a Cytoflor flourescent plate reader with excitation at 360 nm and emission read at 450 nm. For optimal measurement of flourescent intensity, black plates with clear bottoms were used.

Results and Discussion:

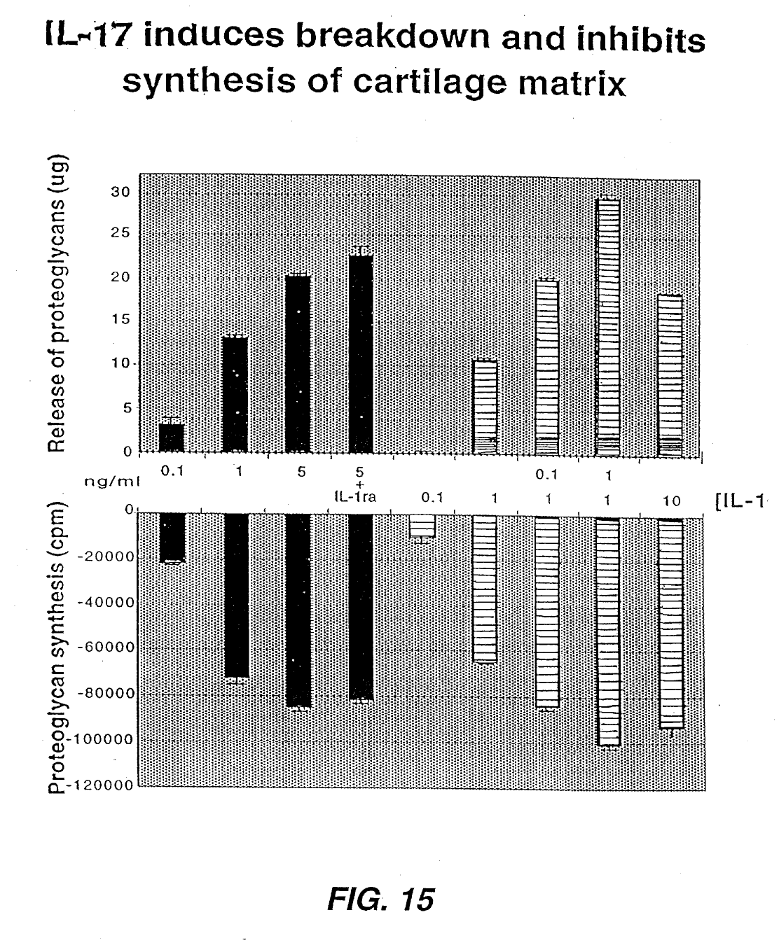

IL-17 was observed to both increase the release of and decrease the synthesis of proteoglycans (FIG. 15). Moreover, this effect was additive to the effect observed from IL-1α (FIG. 15).

In conclusion, IL-17 likely contributes to loss of articular cartilage in arthritic joints, and thus inhibition of its activity might limit inflammation and cartilage destruction. IL-1 and IL-17 have similar yet distinctive activities, due to their use of different receptors and overlapping downstream signaling mechanisms.

Given the finding of the potent catabolic effects of IL-17 on articular cartilage explants, antagonists may be useful for the treatment of inflammatory conditions and cartilage defects such as arthritis. Finally, it is well known that growth factors can have biphasic effects and that diseased tissue can respond differently than normal tissue to a given factor in vivo. For these reasons, antagonists of IL-17 may be useful for the treatment of inflammatory conditions and joint disorders such as arthritis.

### Example 7

### Expression of IL-17 and LIF Antagonist Polypeptides and Antibodies in E. coli

This example illustrates the preparation of unglycosylated forms of IL-17 and LIF antagonist polypeptides and antibodies (hereinafter “antagonists”) by recombinant expression in E. coli.

The DNA sequence encoding the full-length antagonist or a fragment or variant thereof is initially amplified using selected PCR primers. The primers should contain restriction enzyme sites which correspond to the restriction enzyme sites on the selected expression vector. A variety of expression vectors may be employed. An example of a suitable vector is pBR322 (derived from E. coli; see Bolivar et al., Gene, 2:95 (1977)) which contains genes for ampicillin and tetracycline resistance. The vector is digested with restriction enzyme and dephosphorylated. The PCR amplified sequences are then ligated into the vector. The vector will preferably include sequences which encode for an antibiotic resistance gene, a trp promoter, a polyhis leader (including the first six STII codons, polyhis sequence, and enterokinase cleavage site), the antagonists coding region, lambda transcriptional terminator, and an argU gene.

The ligation mixture is then used to transform a selected E. coli strain using the methods described in Sambrook et al., supra. Transformants are identified by their ability to grow on LB plates and antibiotic resistant colonies are then selected. Plasmid DNA can be isolated and confirmed by restriction analysis and DNA sequencing.

Selected clones can be grown overnight in liquid culture medium such as LB broth supplemented with antibiotics. The overnight culture may subsequently be used to inoculate a larger scale culture. The cells are then grown to a desired optical density, during which the expression promoter is turned on.

After culturing the cells for several more hours, the cells can be harvested by centrifugation. The cell pellet obtained by the centrifugation can be solubilized using various agents known in the art, and the solubilized antagonists polypeptide can then be purified using a metal chelating column under conditions that allow tight binding of the polypeptide.

### Example 8

### Expression of IL-17 and LIF Antagonist Polypeptides and Antibodies in Mammalian Cells

This example illustrates preparation of glycosylated forms of IL-17 and LIF antagonist polypeptides and antibodies (hereinafter “antagonists”) by recombinant expression in mammalian cells.

The vector, pRK5 (see EP 307,247, published Mar. 15, 1989), may be employed as the expression vector. Optionally, the DNA encoding the IL-17 and LIF antagonist polypeptide is ligated into pRK5 with selected restriction enzymes to allow insertion of the antagonist-encoding DNA using ligation methods such as described in Sambrook et al., supra. The resulting vector is called pRK5-antagonist.

In one embodiment, the selected host cells may be 293 cells. Human 293 cells (ATCC CCL 1573) are grown to confluence in tissue culture plates in medium such as DMEM supplemented with fetal calf serum and optionally, nutrient components and/or antibiotics. About 10 μg pRK5-antagonist DNA is mixed with about 1 μg DNA encoding the VA RNA gene, Thimmappaya et al., Cell, 31:543 (1982), and dissolved in 500 μl of 1 mM Tris-HCl, 0.1 mM EDTA, 0.227 M CaCl₂. To this mixture is added, dropwise, 500 μl of 50 mM HEPES (pH 7.35), 280 mM NaCl, 1.5 mM NaPO₄, and a precipitate is allowed to form for 10 minutes at 25° C. The precipitate is suspended and added to the 293 cells and allowed to settle for about four hours at 37° C. The culture medium is aspirated off and 2 ml of 20% glycerol in PBS is added for 30 seconds. The 293 cells are then washed with serum free medium, fresh medium is added and the cells are incubated for about 5 days.

Approximately 24 hours after the transfections, the culture medium is removed and replaced with culture medium (alone) or culture medium containing 200 μCi/ml ³⁵S-cysteine and 200 μCi/ml ³⁵S methionine. After a 12-hour incubation, the conditioned medium is collected, concentrated on a spin filter, and loaded onto a 15% SDS gel. The processed gel may be dried and exposed to film for a selected period of time to reveal the presence of antagonist. The cultures containing transfected cells may undergo further incubation (in serum free medium) and the medium is tested in selected bioassays.

In an alternative technique, antagonist-encoding DNA may be introduced into 293 cells transiently using the dextran sulfate method described by Somparyrac et al., Proc. Natl. Acad. Sci., 12:7575 (1981). 293 cells are grown to maximal density in a spinner flask and 700 μg pRK5-antagonist DNA is added. The cells are first concentrated from the spinner flask by centrifugation and washed with PBS. The DNA-dextran precipitate is incubated on the cell pellet for four hours. The cells are treated with 20% glycerol for 90 seconds, washed with tissue culture medium, and re-introduced into the spinner flask containing tissue culture medium, 5 μg/ml bovine insulin and 0.1 μg/ml bovine transferrin. After about four days, the conditioned media is centrifuged and filtered to remove cells and debris. The sample containing expressed antagonist can then be concentrated and purified by any selected method, such as dialysis and/or column chromatography.

In another embodiment, antagonist can be expressed in CHO cells. The pRK5-antagonist vector can be transfected into CHO cells using known reagents such as CaPO₄ or DEAE-dextran. As described above, the cell cultures can be incubated, and the medium replaced with culture medium (alone) or medium containing a radiolabel such as ³⁵S-methionine. After determining the presence of the antagonist, the culture medium may be replaced with serum free medium. Preferably, the cultures are incubated for about 6 days, and then the conditioned medium is harvested. The medium containing the expressed antagonist can then be concentrated and purified by any selected method.

Epitope-tagged antagonist may also be expressed in host CHO cells. The antagonist-encoding DNA may be subcloned out of the pRK5 vector. The subclone insert can undergo PCR to fuse in frame with a selected epitope tag such as a poly-his tag into a Baculovirus expression vector. The poly-his tagged antagonist-encoding DNA insert can then be subcloned into a SV40 driven vector containing a selection marker such as DHFR for selection of stable clones. Finally, the CHO cells can be transfected (as described above) with the SV40 driven vector. Labeling may be performed, as described above, to verify expression. The culture medium containing the expressed poly-H is tagged antagonist can then be concentrated and purified by any selected method, such as by Ni²⁺-chelate affinity chromatography.

### Example 9

### Expression of a IL-17 and LIF Antagonist Polypeptides and Antibodies in Yeast

The following method describes recombinant expression of IL-17 and LIF antagonist polypeptides or antibodies in yeast.

First, yeast expression vectors are constructed for intracellular production or secretion of IL-17 and LIF antagonist polypeptide or antibody from the ADH2/GAPDH promoter. DNA encoding the IL-17 and LIF antagonist polypeptide or antibody of interest, a selected signal peptide and the promoter is inserted into suitable restriction enzyme sites in the selected plasmid to direct intracellular expression of the IL-17 and LIF antagonist polypeptide or antibody. For secretion, DNA encoding the IL-17 and LIF antagonist polypeptide or antibody can be cloned into the selected plasmid, together with DNA encoding the ADH2/GAPDH promoter, the yeast alpha-factor secretory signal/leader sequence, and linker sequences (if needed) for expression of the IL-17 and LIF antagonist polypeptide or antibody.

Yeast cells, such as yeast strain AB110, can then be transformed with the expression plasmids described above and cultured in selected fermentation media. The transformed yeast supernatants can be analyzed by precipitation with 10% trichloroacetic acid and separation by SDS-PAGE, followed by staining of the gels with Coomassie Blue stain.

Recombinant IL-17 and LIF antagonist polypeptide or antibody can subsequently be isolated and purified by removing the yeast cells from the fermentation medium by centrifugation and then concentrating the medium using selected cartridge filters. The concentrate containing the IL-17 and LIF antagonist polypeptide or antibody may further be purified using selected column chromatography resins.

### Example 10

### Expression of IL-17 and LIF Antagonist Polypeptides and Antibodies in Baculovirus-Infected Insect Cells

The following method describes recombinant expression IL-17 and LIF antagonist polypeptides and antibodies in Baculovirus-infected insect cells.

The DNA encoding the IL-17 or LIF antagonist polypeptide or antibody is fused upstream of an epitope tag contained within a baculovirus expression vector. Such epitope tags include poly-his tags and immunoglobulin tags (like Fc regions of IgG). A variety of plasmids may be employed, including plasmids derived from commercially available plasmids such as pVL1393 (Novagen). Briefly, the PRO1031- or PRO1122-encoding DNA or the desired portion of the IL-17 or LIF antagonist polypeptide or antibody (such as the sequence encoding the region which interacts with or binds the IL-17 or LIF ligand or receptor) is amplified by PCR with primers complementary to the 5′ and 3′ regions. The 5′ primer may incorporate flanking (selected) restriction enzyme sites. The product is then digested with those selected restriction enzymes and subcloned into the expression vector.

Recombinant baculovirus is generated by co-transfecting the above plasmid and BaculoGold™ virus DNA (Pharmingen) into Spodoptera frugiperda (“Sf9”) cells (ATCC CRL 1711) using lipofectin (commercially available from GIBCO-BRL). After 4 to 5 days of incubation at 28° C., the released viruses are harvested and used for further amplifications. Viral infection and protein expression is performed as described by O'Reilley et al., Baculovirus Expression vectors: A Laboratory Manual, Oxford: Oxford University Press (1994).

Expressed poly-his tagged IL-17 or LIF antagonist polypeptide or antibody can then be purified, for example, by Ni²⁺-chelate affinity chromatography as follows. Extracts are prepared from recombinant virus-infected Sf9 cells as described by Rupert et al., Nature, 362:175-179 (1993). Briefly, Sf9 cells are washed, resuspended in sonication buffer (25 mL Hepes, pH 7.9; 12.5 mM MgCl₂; 0.1 mM EDTA; 10% Glycerol; 0.1% NP-40; 0.4 M KCl), and sonicated twice for 20 seconds on ice. The sonicates are cleared by centrifugation, and the supernatant is diluted 50-fold in loading buffer (50 mM phosphate, 300 mM NaCl, 10% Glycerol, pH 7.8) and filtered through a 0.45 μm filter. A Ni²⁺-NTA agarose column (commercially available from Qiagen) is prepared with a bed volume of 5 mL, washed with 25 mL of water and equilibrated with 25 mL of loading buffer. The filtered cell extract is loaded onto the column at 0.5 mL per minute. The column is washed to baseline A₂₈₀ with loading buffer, at which point fraction collection is started. Next, the column is washed with a secondary wash buffer (50 mM phosphate; 300 mM NaCl, 10% Glycerol, pH 6.0), which elutes nonspecifically bound protein. After reaching A₂₈₀ baseline again, the column is developed with a 0 to 500 mM Imidazole gradient in the secondary wash buffer. One mL fractions are collected and analyzed by SDS-PAGE and silver staining or western blot with Ni²⁺-NTA-conjugated to alkaline phosphatase (Qiagen). Fractions containing the eluted His₁₀-tagged IL-17 or LIF antagonist polypeptides or antibodies are pooled and dialyzed against loading buffer.

Alternatively, purification of the IgG tagged (or Fc tagged) the IL-17 or LIF antagonist polypeptide or antibody can be performed using known chromatography techniques, including for instance, Protein A or protein G column chromatography.

### Example 11

### Preparation of Antibodies that Bind IL-17 or LIF Polypeptides or Receptors

This example illustrates the preparation of monoclonal antibodies which can specifically bind to IL-17 or LIF polypeptides or receptors.

Techniques for producing the monoclonal antibodies are known in the art and are described, for instance, in Goding, supra. Immunogens that may be employed include purified to IL-17 or LIF polypeptides or receptors, fusion proteins containing a to IL-17 or LIF polypeptides or receptors, and cells expressing recombinant to IL-17 or LIF polypeptides or receptors on the cell surface. Selection of the immunogen can be made by the skilled artisan without undue experimentation.

Mice, such as Balb/c, are immunized with the to IL-17 or LIF polypeptide or receptor immunogen which has been emulsified in complete Freund's adjuvant and injected subcutaneously or intraperitoneally in an amount from 1-100 micrograms. Alternatively, the immunogen is emulsified in MPL-TDM adjuvant (Ribi Immunochemical Research, Hamilton, Mont.) and injected into the animal's hind foot pads. The immunized mice are then boosted 10 to 12 days later with additional immunogen emulsified in the selected adjuvant. Thereafter, for several weeks, the mice may also be boosted with additional immunization injections. Serum samples may be periodically obtained from the mice by retro-orbital bleeding for testing in ELISA assays to detect anti-IL-17, anti-IL-17R or anti-LIF, anti-LIFR polypeptide antibodies.

After a suitable antibody titer has been detected, the animals “positive” for antibodies can be injected with a final intravenous injection of IL-17, IL-17R or LIF or LIFR polypeptide. Three to four days later, the mice are sacrificed and the spleen cells are harvested. The spleen cells are then fused (using 35% polyethylene glycol) to a selected murine myeloma cell line such as P3X63AgU.1, available from ATCC, No. CRL 1597. The fusions generate hybridoma cells which can then be plated in 96 well tissue culture plates containing HAT (hypoxanthine, aminopterin, and thymidine) medium to inhibit proliferation of non-fused cells, myeloma hybrids, and spleen cell hybrids.

The hybridoma cells will be screened in an ELISA for reactivity against IL-17, IL-17R or LIF, LIFR polypeptide. Determination of “positive” hybridoma cells secreting the desired monoclonal antibodies against an IL-17, IL-17R or LIF, LIFR polypeptide is within the skill in the art.

The positive hybridoma cells can be injected intraperitoneally into syngeneic Balb/c mice to produce ascites containing the anti-IL-17, anti-IL-17R or anti-LIF, anti-LIFR polypeptide monoclonal antibodies. Alternatively, the hybridoma cells can be grown in tissue culture flasks or roller bottles. Purification of the monoclonal antibodies produced in the ascites can be accomplished using ammonium sulfate precipitation, followed by gel exclusion chromatography. Alternatively, affinity chromatography based upon binding of antibody to protein A or protein G can be employed.

### Example 12

### Purification of IL-17, IL-17R or LIF, LIFR Antagonist Specific Antibodies

Native or recombinant IL-17, IL-17R, LIF or LIFR antagonists (polypeptides or antibodies) may be purified by a variety of standard techniques in the art of protein purification. For example, pro-IL-17, pro-IL-17R, pro-LIF or pro-LIFR polypeptide, mature IL-17, IL-17R, LIF or LIFR polypeptide or antibody, or pre-IL-17, pre-IL-17R, pre-LIF or pre-LIFR polypeptide or antibody is purified by immunoaffinity chromatography using antibodies specific for the IL-17, IL-17R, LIF or LIFR antagonist of interest. In general, an immunoaffinity column is constructed by covalently coupling the anti-IL-17, anti-IL-17R, anti-anti-IL-17, anti-anti-IL-17R, anti-LIF, anti-LIFR, anti-anti-LIF or anti-anti-LIFR antibody to an activated chromatographic resin.

Polyclonal immunoglobulins are prepared from immune sera either by precipitation with ammonium sulfate or by purification on immobilized Protein A (Pharmacia LKB Biotechnology, Piscataway, N.J.). Likewise, monoclonal antibodies are prepared from mouse ascites fluid by ammonium sulfate precipitation or chromatography on immobilized Protein A. Partially purified immunoglobulin is covalently attached to a chromatographic resin such as CnBr-activated SEPHAROSE® (Pharacia LKB Biotechnology). The antibody is coupled to the resin, the resin is blocked, and the derivative resin is washed according to the manufacturer's instructions.

Such an immunoaffinity column is utilized in the purification of IL-17, IL-17R, LIF or LIFR polypeptide or antibody by preparing a fraction of cells containing IL-17, IL-17R, LIF or LIFR polypeptide or antibody in a soluble form. This preparation is derived by solubilization of the whole cell or of a subcellular fraction obtained via differential centrifugation by the addition of detergent or by other methods well known in the art. Alternatively, soluble IL-17, IL-17R, LIF or LIFR polypeptide or antibody containing a signal sequence may be secreted in useful quantity into the medium in which the cells are grown.

A soluble IL-17, IL-17R, LIF or LIFR polypeptide or antibody-containing preparation is passed over the immunoaffinity column, the and column is washed under conditions that allow the preferential absorbance of IL-17, IL-17R, LIF or LIFR polypeptide or antibody (e.g., high ionic strength buffers in the presence of detergent). Then, the column is eluted under conditions that disrupt the binding of the resin-linked antibody to the IL-17, IL-17R, LIF or LIFR polypeptide or antibody (e.g., a low pH buffer such as approximately pH 2-3, or a high concentration of a chaotrope such as urea or thiocyanate ion), and IL-17, IL-17R, LIF or LIFR polypeptide or antibody is collected.

### REFERENCES

Aarvak, T., M. Chabaud, P. Miossec, and J B. Natvig. 1999. IL-17 is produced by some proinflammatory Th1/Th0 cells but not by Th2 cells. J. Immunology 162: 1246-1251.
Amin, A. R., and Abramson, S. B. 1998. The role of nitric oxide in articular cartilage breakdown in osteoarthritis. Curr. Opinion in Rheum. 10: 263-268.
Arend, W. P. and J-M. Dayer. 1990. Cytokines and cytokine inhibitors or antagonists in rheumatoid arthritis. Arthritis Rheum. 33: 305-315.
Arend W. P. and J.-M. Dayer. 1995. Inhibition of the production and effects of interleukin-1 and tumor necrosis factor-α in rheumatoid arthritis. Arthritis Rheum. 38:151.
Attur, M. G., Patel, R. N., Abramson, S. B. and A. R. Amin. Interleukin-17 up-regulation of nitric oxide production in human osteoarthritis cartilage. 1997. Arthritis Rheum. 40: 1050-1053.
Bresnihan, B., J. M. Alvaro-Gracia, M. Cobby, M. Doherty, Z. Domljan, P. Emery, G. Nuki, K. Pavelka, R. Rau, and P. Musikic. 1998. Treatment of rheumatoid arthritis with recombinant human interleukin-1 receptor antagonist. Arthritis Rheum. 41: 2196.
Campbell, I. K., P. Waring, U. Novak, J. A. Hamilton. 1993. Production of leukemia inhibitory factor by human articular chondrocytes and cartilage in response to interleukin-1 and tumor necrosis factor alpha. Arthritis Rheum. 36: 790-4.
Chabaud, M., F. Fossiez, J-L Taupin, and P. Miossec. 1998. Enhancing effect of IL-17 on IL-1-induced IL-6 and leukemia inhibitory factor production by rheumatoid arthritis synoviocytes and its regulation by Th2 cytokines. J. Immunol. 161: 409-414.
Chabaud, M., J. M. Durand, N. Buchs, F. Fossiez, G. Page, L. Frappart, and P. Miossec. 1999. Human interleukin-17: a T-cell derived proinflammatory cytokine produced by the rheumatoid synovium. Arthritis Rheum. 42: 963-970.
Dudler, J., Renggli-Zulliger, N., Busso, N., Lotz, M, and A. So. 2000. Effect of interleukin 17 on proteoglycan degradation in murine knee joints. Ann. Rheum. Dis. 59:529-532.
Elliott, M. J., R. N. Maini, M. Feldmann, A. Long-Fox, P. Charles, P. Katsikis, F. M. Brennan, J. Walker, H. Bijl, J. Ghrayeb, and J. Woody. 1993. Treatment of rheumatoid arthritis with chimeric monoclonal antibodies to tumor necrosis factor-α. Arthritis Rheum. 36: 1681.
Farndale, R. W. and Buttle, A. J. 1986. Improved quantitation and discrimination of sulphated glycosaminoglycans by use of dimethylmethylene blue. Biochem. Biophys. Acta 883: 173-177
Farrell, A. J., D. Blake, R. M. J. Palmer, S. Moncada, 1992. Increased concentrations of nitrite in synovial fluid and serum samples suggest increased nitric oxide synthesis in rheumatic diseases. Ann. Rheum. Dis. 51: 1219-1222.
Firestein G. S., J. M. Alvaro-Gracia, R. Maki, J. M. A. G. Alvaro-Gracia. 1990. Quantitative analysis of cytokine gene expression in rheumatoid arthritis. J. Immunol. 144: 3347-53.
Fossiez, F., O. Djossou, P. Chomarat, L. Flores-Romo, S. Ait-Yahia, C. Maat, J. J. Pin, P. Garrone, E. Garcia, S. Saeland, D. Blanchard, C. Gaillard, B. Das Mahapatra, E. Rouvier, P. Golstein, J. Banchereau, and S. Lebecque. 1996. T-cell interleukin-17 induces stromal Cells to produce proinflammatory and hematopoietic cytokines. J. Exp. Med. 183: 2593-2603.
Hamilton, J. A., P. M. Waring, E. L. Filonzi. 1993. Induction of leukemia inhibitory factor in human synovial fibroblasts by IL-1 and tumor necrosis factor-alpha. J. Immunol. 150: 1496-502.
Hartmann C. and C. J. Tabin. 2000. Dual roles of Wnt signaling during chondrogenesis in the chicken limb. Development 127: 3141-3159.
Hughes, C. E., Little, C. B., Buttner, F. H., Bartnik, E., and Caterson, B. 1998. Differential expression of aggrecanase and matrix metalloproteinase activity in chondrocytes isolated from bovine and porcine articular cartilage. J. Biol. Chem. 273: 30576-30582.
Hui, W., M. C. Bell, G. J. Carroll, M. Layton. 1998. Modulation of cartilage proteoglycan metabolism by LIF binding protein. Cytokine 10: 220-226.
Joosten, L. A. B., M. M. A. Helsen, F. A. J. van de Loo, W. B. van den berg, 1996. Anticytokine treatment of established type II collagen-induced arthritis in DBA/1 mice: a comparative study using anti-TNFalpha, anti-IL-1alpha/beta, and IL-1Ra. Arthritis & Rheumatism 39(5):797-809.
Joosten, L. A. B., M. M. A. Helsen, T. Saxne, F. A. J. van de Loo, D. Heinegard, W. B. van den Berg. 1999. IL-1α blockade prevents cartilage and bone destruction in murine type II collagen-induced arthritis, whereas TNF-α blockade only ameliorates joint inflammation. J. Immunol. 163: 5049-5055.
Jovanovic, D. V., J. A. DiBattista, J. Martel-Pelletier, F. C. Jolicoeur, Y. He, M. Zhang, F. Mineau, and J-P Pelletier. 1998. IL-17 stimulates the production and expression of proinflammatory cytokines, IL-beta and TNF-alpha, by human macrophages. J. Immunol. 160: 3513-21.
Jovanovic, D. V., J. Martel-Pelletier, H., J. A. Di Battista, F. Mineau, F-C. Jolicoeur, M. Benderdour, and J-P Pelletier. 2000. Stimulation of 92-kd gelatinase (matrix metalloproteinase 9) production by interleukin-17 in human monocyte/macrophages. Arthritis Rheum. 43: 1134-1144.
Kim, K. J., M. Alphonso, C. H. Schmelzer and D. Lowe. 1992. Detection of human leukemia inhibitory factor by monoclonal antibody based ELISA. J. Immunol. Methods 156: 9-17.
Kingsley, G. H. and G. S. Panayi. 1997. Joint destruction in rheumatoid arthritis: biological bases. Clin. Exp. Rheumatol. 15: S3-14.
Kong, Y-Y, U. Felge, I. Sarosi, B. Bolon, A. Tafuri, S. Morony, C. Capparelli, J. Li, R. Elliott, S. McCabe, T. Wong, G. Campagnuolo, E. Moran, E. R. Bogoch, G. Van, L. T. Nguyen, P. S. Ohasi, D. L. Lacey, E. Fish, W. J. Boyle, and J. M. Penninger. 1999. Activated T cells regulate bone loss and joint destruction in adjuvant arthritis through osteoprotegerin ligand. Nature. 402: 304-308.
Kotake, S., N. Udagawa, N. Takahashi, K. Matsuzaki, K. Itoh, S. Ishiyama, S. Saito, K. Inoue, N. Kamatani, M. T. Gillespie, T. J. Martin, and T. Suda. 1999. IL-17 in synovial fluids from patients with rheumatoid arthritis is a potent stimulator of osteoclastogenesis. J. Clinical Invest. 103: 1345-1351.
Lark, M. W., E. K. Bayne, J. Flanagan, C. F. Harper, L. A. Hoerner, N. I. Hutchinson, I. Singer, S. A. Donatelli, J. R. Weidner, H. R. Williams, R. A. Mumford, and L. S. Lohmander. 1997. Aggrecan degradation in human cartilage. Evidence for both matrix metalloproteinase and aggrecanase activity in normal, osteoarthritic, and rheumatoid joints. J. Clin Invest. 100: 93-106.
Lendeckel U, B. Scholz, M. Arndt, K. Frank, A. Spiess, H. Chen, B. P. Roques, S. Ansorge. 2000. Inhibition of alanyl-aminopeptidase suppresses the activation-dependent induction of glycogen synthase kinase-3β (GSK-3beta) in human T cells. Biochem. Biophys. Res. Commun. 273:62-5.
Lendeckel, U., M. Arndt, K. Frank, A. Spiess, D. Reinhold, and S. Ansorge. 2000. Modulation of Wnt-5A expression by actinonin: linkage of APN to the Wnt-pathway. Adv. Exp Med Biol. 477:35-41.
Little C. B., C. R. Flannery, C. E. Hughes, J. S. Mort, P. J. Roughley, C. Dent, and B. Caterson. 1999. Aggrecanase versus matrix metalloproteinases in the catabolism of the interglobular domain of aggrecan in vitro. Biochem J. 344:61-8.
Lohmander, L. S., P. J. Neame, and J. D. Sandy. 1993. The structure of aggrecan fragments in human synovial fluid. Evidence that aggrecanase mediates cartilage degradation in inflammatory joint disease, joint injury, and osteoarthritis, Arthritis Rheum. 36: 1214-22.
Lotz, M., T. Moats, P. M. Villiger. 1992. Leukemia inhibitory factor is expressed in cartilage and synovium and can contribute to the pathogenesis of arthritis. J. Clin. Invest. 90: 888-96.
Lubberts, E., Joosten, L. A. B., van de Loo F. A. J., L. A. M. van den Bersselaar, W. B. van den Berg. 2000. Reduction of interleukin-17-induced inhibition of chondrocyte proteoglycan synthesis in intact murine articular cartilage by interleukin 4. Arthritis Rheum. 43: 1300-1306.
Martel-Pelletier, J., Di Battista, J. A., Lajeunesse, D., and Pelletier, J.-P. 1998. IGF/IGFBP axis in cartilage and bone in osteoarthritis pathogenesis. Inflamm. Res. 47: 90-100.
Martel-Pelletier J., N. Alaaeddine, J-P Pelletier. Cytokines and their role in the pathophysiology of osteoarthritis. 1999. Front. Biosci. 4: d694-703.
Martel-Pelletier, J., F. Mineau, D. Jovanovic, J. A. DiBattista, and J-P Pelletier. 1999. Mitogen-activated protein kinase and nuclear factor kB together regulate interleukin-17-induced nitric oxide production in human osteoarthritic chondrocytes. Arthritis Rheum. 42: 2399-2409.
Murrell, G. A. C., D. Jang, R. J. Williams. 1995. Nitric oxide activates metalloprotease enzymes in articular cartilage. Biochem. Biophys. Res. Commun. 206: 15-21.
Palmer R M, Hickery M S, Charles I G, Moncada S, and Bayliss M T. 1993. Induction of nitric oxide synthase in human chondrocytes. Biochem. Biophys. Res. Commun. 193: 398-405.
Panayi, G. S., J. S. Lanchbury, and G. H. Kingsley. 1992. The importance of the T cell in initiating and maintaining the chronic synovitis of rheumatoid arthritis. Arthritis Rheum. 35: 729-735.
Pelletier, J-P, D. Jovanovic, J. C. Fernandes, P. Manning, J. R. Connor, M. G. Currie, et al. 1998. Reduced progression of experimental osteoarthritis in vivo by selective inhibition of inducible nitric oxide synthase. Arthritis Rheum 41: 1275-86.
Pelletier J, D. Jovanovic, J. C. Fernandes, P. Manning, Connor J R, M. G. Currie, J. Martel-Pelletier, 1999. Reduction in the structural changes of experimental osteoarthritis by a nitric oxide inhibitor. Osteoarthritis Cartilage 7(4): 416-8.
Renoux, M. P. Hilliquin, L. Galoppin, I. Florentin, C. J. Menkes. 1996. Release of mast cell mediators and nitrites into knee joint fluid in osteoarthritis: comparison with articular chondrocalcinosis and rheumatoid arthritis. Osteoarthritis Cartilage 4:175-179.
Sakiniene, E., T. Bremell, and A. Tarkowski. 1997. Inhibition of nitric oxide synthase (NOS) aggravates Staphylococcus aureus septicaemia and septic arthritis. Clin. Exp. Immunol. 110:370-7.
Sandy, J. D., C. R. Flannery, P. J. Neame, L. S. Lohmander. 1992. The structure of aggrecan fragments in human synovial fluid. Evidence for the involvement in osteoarthritis of a novel proteinase which cleaves the Glu373-Ala 374 bond of the interglobular domain. J. Clin. Invest. 89: 1512-1516.
Schwarzenberger, P., V. La Russa, A. Miller, P. Ye, W. Huang, A. Zieske, S, Nelson, G. J. Bagby, D. Stoltz, R. L. Mynatt, M. Spriggs, and J. Kolls. 1998. IL-17 stimulates granulopoiesis in mice: Use of alternate, novel gene therapy-derived method for in vivo evaluation of cytokines. J. Immunol. 161: 6383-6389.
Shalom-Barak T., Quach, J., and M. Lotz, 1999. Interleukin-17 induced gene expression in articular chondrocytes is associated with activation of mitogen-activated protein kinases and NF-κB. J. Biol. Chem. 273: 27467-473.
Stefanovic-Racic, M. T. I. Morales, D. Taskiran, L. A. McIntyre, C. H. Evans. 1996. The role of nitric oxide in proteoglycan turnover by bovine articular cartilage organ cultures. J. Immunol. 156:1213-20.
Stadler J, M. Stefanovic-Racic, T. R. Billiar, R. D. Curran, L. A. McIntyre, H. I. Georgescu, R. L. Simmons, and C. H. Evans. 1991. Articular chondrocytes synthesize nitric oxide in response to cytokines and lipopolysaccharide. J. Immunol. 147:3915-20.
Stichtenoth D. O., and J. C. Frolich. 1998. Nitric oxide and inflammatory joint diseases. Br. J. Rheumatol. 37: 246-57.
Smith R. L. 1999. Degradative enzymes in osteoarthritis. Front. Biosci. 4:d704-712).
Taskiran, D., M. Stefanovic-Racic, H. I. Georgescu, and C. H. Evans. 1994. Nitric oxide mediates suppression of cartilage proteoglycan synthesis by interleukin-1. Biochem. Biophys. Res. Commun. 200: 142.
Tyler, J. A., Bolis, S., Dingle, J. T. and J. F. S. Middleton. 1992. Mediators of matrix catabolism. In Articular Cartilage and Osteoarthritis, edited by K. Kuettner et al. Raven Press, Ltd., New York. p251-264.
Ulfgren, A-K., S. Lindblad, K. Klareskog, J. Andersson, U. Anderson. 1995. Detection of cytokine producing cells in the synovial membrane from patients with rheumatoid arthritis. Ann. Rheum. Dis. 54: 654-67.
van de Loo F A, Amtz O J, van Enckevort F H, van Lent P L, van den Berg W B. 1998. Reduced cartilage proteoglycan loss during zymosan-induced gonarthritis in NOS2-deficient mice and in anti-interleukin-1-treated wild-type mice with unabated joint inflammation. Arthritis Rheum. 41:634-46.
Villiger, P. M. Y. Geng, M. Lotz. 1993. Induction of cytokine expression by leukemia inhibitory factor. J. Clin. Invest. 91: 1575-81.
Ziolkowska, M., A. Koc, G. Luszczykiewicz, K. Ksiezopolska-Pietrzak, E. Klimczak, H. Chwalinska-Sadowska, and W. Maslinski. 2000. High levels of IL-17 in rheumatoid arthritis patients: IL-15 triggers in vitro IL-17 production via cyclosporin A-sensitive mechanism. J. Immunol. 164: 2832-2838.

What is claimed is:

1. A method of treating cartilage damaged from a cartilagenous disorder comprising contacting the cartilage with an effective amount of an antagonist to IL-17 or LIF.

2. The method of claim 1, wherein the IL-17 or LIF antagonist is an anti-IL-17 or anti-LIF antibody.

3. The method of claim 1, wherein the cartilage is articular cartilage.

4. The method of claim 1, wherein the cartilagenous disorder is a degenerative cartilagenous disorder.

5. The method of claim 4, wherein the degenerative cartilagenous disorder is arthritis.

6. The method of claim 5, wherein the arthritis is rheumatoid arthritis.

7. The method of claim 4, wherein the degenerative cartilagenous disorder is osteoarthritis.

8. The method of claim 1, wherein the cartilage is contained in a mammal and the effective amount is a therapeutically effective amount.

9. The method of claim 8, wherein the antagonist to IL-17 or LIF is administered by direct injection into an afflicted cartilagenous region or joint.

10. The method of claim 1 wherein the cartilagenous disorder results from injury.

11. The method of claim 10 wherein the type of injury is a microdamage or blunt trauma, a chondral fracture, an osteochondral fracture or damage to meniscus, tendon or ligament.

12. The method of claim 11, wherein the injury is the result of excessive mechanical stress or other biomechanical instability resulting from a sports injury or obesity.

13. The method of claim 1, wherein the IL-17 or LIF antagonist further comprises a carrier, excipient or stabilizer.

14. The method of claim 1 wherein the contacting is combined with a standard surgical technique.

15. The method of claim 1 wherein the IL-17 or LIF antagonist is combined with an effective amount of at least one cartilage agent.

16. The method of claim 15 wherein the cartilage agent is selected from the group consisting of a peptide growth factor, a catabolism antagonist, an osteo-factor, a synovial factor and an anti-inflammatory factor.

17. The method of claim 16 wherein the peptide growth factor is selected from the group consisting of IGFs, PDGF-AA, PDGF-AB, PDGF-BB, BMPs, FGFs, TGF-βs and EGF.

18. The method of claim 16 wherein the catabolism antagonist is selected from the group consisting of IL-1ra, NO inhibitors, ICE inhibitors, agents which inhibit the activity of IL-6, IL-8, IFN-γ, TNF-α, tetracyclines and variants thereof, inhibitors of apoptosis, MMP inhibitors, aggrecanase inhibitors and inhibitors of serine and cysteine proteinases.

19. The method of claim 16 wherein the osteo-factor is selected from the group consisting of bisphosphonates and osteoprotegerin.

20. The method of claim 16 wherein the anti-inflammatory factor is selected from the group consisting of anti-TNF-α, soluble TNF receptors, IL-1ra, soluble IL-1 receptors, IL-4, IL-10 and IL-13.

21. A method of preventing cartilage damage caused by a cartilagenous disorder comprising contacting the cartilage with an effective amount of an IL-17 or LIF antagonist.

22. The method of claim 21 wherein the IL-17 and LIF antagonists are anti-IL-17 and anti-LIF antibodies.

23. The method of claim 21, wherein the cartilage is articular cartilage.

24. The method of claim 21, wherein the cartilagenous disorder is a degenerative cartilagenous disorder.

25. The method of claim 24 wherein the degenerative cartilagenous disorder is arthritis.

26. The method of claim 25 wherein the arthritis is rheumatoid arthritis.

27. The method of claim 25 wherein the arthritis is osteoarthritis.

28. The method of claim 21 wherein the effective amount is a therapeutically effective amount and the cartilage is present in a mammal.

29. The method of claim 28 wherein the IL-17 and LIF antagonist is administered by direct injection into an afflicted cartilagenous region or joint.

30. The method of claim 21 wherein the cartilagenous disorder results from injury.

31. The method of claim 30 wherein the type of injury is a microdamage or blunt trauma, a chondral fracture, an osteochondral fracture or damage to meniscus, tendon or ligament.

32. The method of claim 30, wherein the injury is the result of excessive mechanical stress or other biomechanical instability resulting from a sports injury or obesity.

33. The method of claim 21, wherein the effective amount of IL-17 or LIF antagonist further comprises a carrier, excipient or stabilizer.

34. The method of claim 21, wherein the contacting is combined with a standard surgical technique.

35. The method of claim 21, wherein the IL-17 or LIF antagonist is combined with an effective amount of at least one cartilage agent.

36. The method of claim 35, wherein the cartilage agent is selected from the group consisting of a peptide growth factor, a catabolism antagonist, an osteo-factor, a synovial factor and an anti-inflammatory factor.

37. A method of treating a mammal suffering from a cartilagenous disorder, comprising administering to said mammal a therapeutically effective amount of an antagonist to IL-17 or LIF.

38. The method of claim 37, wherein the IL-17 and LIF antagonists are anti-IL-17 and anti-LIF antibodies.

39. The method of claim 37, wherein the cartilagenous disorder is a degenerative cartilagenous disorder.

40. The method of claim 39, wherein the degenerative cartilagenous disorder is arthritis.

41. A composition of matter comprising an effective amount of IL-17 and LIF antagonist.

42. The composition of claim 41, wherein the IL-17 and LIF antagonists are anti-IL17 and anti-LIF antibodies.

43. The composition of claim 41 further comprising an effective amount of a cartilage agent.

44. The composition of claim 41 further comprising a carrier, excipient or stabilizer.


SEQUENCE LISTING (4 sequences)

SEQ ID NO: 1 - protein, 5 aa, Artificial Sequence
  N-terminal aggrecan cleavage fragment.
ARGSV

SEQ ID NO: 2 - protein, 10 aa, Artificial Sequence
  Ovalbumin-derived antigenic peptide.
FFGVGAKKGC

SEQ ID NO: 3 - protein, 5 aa, Artificial sequence
  sequence is synthesized
ARGSV

SEQ ID NO: 4 - protein, 9 aa, Artificial sequence
  sequence is synthesized
ARGSVIGGC